(19) United States
(12) Patent Application Publication
(10) Pub. No.: US 2006/0198819 A1
(43) Pub. Date: Sep. 7, 2006

(54) Use of galactose oxidase for selective chemical conjugation of protractor molecules to proteins of therapeutic interest

(75) Inventors: Carsten Behrens, Copenhagen N (DK); Patrick William Garibay, Holte (DK); Magali Zundel, Soborg (DK); Niels Kristian Klausen, Gentofte (DK); Soren E. Bjorn, Lyngby (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/339,752

(22) Filed: Jan. 25, 2006

Related U.S. Application Data
(63) Continuation of application No. PCT/DK04/00530, filed on Aug. 9, 2004.

(30) Foreign Application Priority Data
Aug. 8, 2003 (DK) PA 2003 01145
Nov. 5, 2003 (DK) PA 2003 01646

(51) Int. Cl. A61K 38/28; A61K 38/22; A61K 38/37; A61K 38/26; A61K 38/20; A61K 38/19; A61K 38/16; C07K 14/54; C07K 14/53; C07K 14/62; C07K 14/42; C07K 14/51

(52) U.S. Cl. 424/85.1; 424/731; 424/85.2; 514/8; 514/12; 530/350; 530/351; 530/395; 530/397; 530/303; 530/396; 530/370; 530/383; 514/3

(57) ABSTRACT

This invention relates to novel compounds, methods for selective chemical conjugation of protractor molecules and the use thereof for diagnostic and/or therapeutic purposes.

### FIELD OF THE INVENTION

This invention relates to the use of galactose oxidase in combination with terminal galactose-containing glycoproteins (such as, e.g., sialidase treated glycoproteins (asialo glycoproteins)), for selective chemical conjugation of protractor molecules. The sequential enzymatic treatment of natural glycoproteins with sialidases and galactose oxidase produces reactive aldehyde functionalities which chemically can be reacted with nucleophilic conjugation agents to produce novel modified glycoproteins with enhanced pharmacological properties, such as increased circulation half-life or increased distribution volume.

### BACKGROUND OF THE INVENTION

Proteins of biological origin hold great promise as therapeutical agents as they often possess high efficacy and high selectivity towards their natural ligands. Being of biological origin makes them non-toxic and thus safer to use than conventional small molecular drugs, as the organism al ready posses well defined clearing mechanisms as well as metabolic pathways for their disposal. This in combination with the fact, that proteins now can be produced by recombinant DNA techniques in a variety of different expression systems, allowing for large scale production, makes proteins ideal drug candidates. However, therapeutically interesting proteins such as hormones, soluble receptors, cytokines, enzymes, etc., often have short circulation half-life in the body which generally reducing their therapeutically utility.

Therapeutic proteins may be removed from circulation by a number of routes. For some pharmacologically active proteins, there are specific receptors which mediate removal from circulation. Proteins which are glycosylated may be cleared by lectin-like receptors in the liver, which exhibit specificity only for the carbohydrate portion of those molecules. Non-specific clearance by the kidney of proteins and peptides (particularly nonglycosylated proteins and peptides) below about 50 KDa has also been documented. It has been noted that asialo-glycoproteins are cleared more quickly by liver than native glycoproteins or proteins lacking glycosylation (Bocci (1990) Advanced Drug Delivery Reviews 4: 149).

### SUMMARY OF THE INVENTION

A problem solved by the present invention is the prolongation of the circulating half-life of soluble glycoprotein derivatives, thus reducing the quantity of injected material and frequency of injection required for maintenance of therapeutically effective levels of circulating glycoprotein for treatment or prophylaxis. The short in vivo plasma half-life of certain therapeutically glycoproteins is undesirable from the stand point of the frequency and the amount of soluble protein which would be required in treatment or prophylaxis. The present invention provides means to prolong the circulating half-life of such glycoproteins with a conservative but still effective change to the glycoprotein structure and with the substantial maintenance of biological activity.

More particular, the present invention provide a general chemo enzymatic methodology for the modification of glycanes (in particular asparagines- or N-linked glycanes) of glycoproteins, in order to improve or enhance their pharmaceutically properties. The method involves optional treatment of the glycoprotein with sialidases (neuraminidases) in order to remove any terminal sialic acid, and oxidization of the exposed galactose residues on the glycoprotein with galactose oxidase in the presence of a hydrogen peroxide scavenger such as, e.g., catalase or horseradish peroxidases. When preformed under appropriate conditions, the galactose oxidase treatment provides glycoproteins, with reactive aldehyde functionalities on the glycan termini, which subsequently can be chemically reacted with nucleophilic reagents to produce glycoconjugates. With glycoproteins which contain a high content of glycans with terminal galactose residues, the optional treatment with sialidases can be omitted.

Thus in a first aspect, the present invention provides a method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded thereto, the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality; and
(b) contacting the glycoprotein product produced in step (a) with a reactant X capable of reacting with an aldehyde group; wherein reactant X comprises a protractor group to create a conjugate represented by the formula (glycoprotein)-(protractor group).

In a second aspect, the present invention provides a method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded to the thereto through a linking moiety; the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality;
(b) contacting the glycoprotein product produced in step (a) with a reactant X capable of reacting with an aldehyde group, wherein reactant X comprises a linking moiety further comprising a second reactive, optionally protected, group to create a conjugate of the glycoprotein and the linking moiety; and
(c) contacting the product of step (b) with a protractor group capable of reacting with the second reactive group of the linking moiety to create a conjugate represented by the formula (glycoprotein)-(linking moiety)-(protractor group).

In one embodiment of the invention the method further comprising step (a1) and step (a2) to be carried out before step (a) or step (c) of the method, respectively:
(a1) contacting a glycoprotein with one or more of sialidases, galacosidases, N-acetylhexosaminidases, fucosidases, mannosidases, endo H and endo F3 to create a glycoprotein where part of the glycan structure is removed,
(a2) contacting the product of step (a1) with a galactosyltransferase and a galactose substrate to create a glycoprotein with at least one terminal residue of galactose or a derivative thereof.

In one embodiment of the invention the method further comprises a first step (a3) to be carried out before step (a), in which:

(a3) contacting a glycoprotein having at least one terminal sialic acid residue with sialidase or another reagent capable of removing sialic acid from the glycans to create an asialo glycoprotein comprising at least one terminal galactose or derivative thereof.

In a third aspect, the present invention provides a glycoprotein conjugate having increased in vivo plasma half-life compared to the non-conjugated glycoprotein, said conjugate comprising a glycoprotein having at least one terminal galactose or derivative thereof, and a protractor group covalently bonded thereto, optionally through a linking moiety.

In a futher aspect, the present invention provides a glycoprotein conjugate obtainable by a method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded thereto, the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality; and
(b) contacting the glycoprotein product produced in step (a) with a reactant X capable of reacting with an aldehyde group; wherein reactant X comprises a protractor group to create a conjugate represented by the formula (glycoprotein)-(protractor group).

In a futher aspect, the present invention provides a glycoprotein conjugate obtainable by a method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded to the thereto through a linking moiety; the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality;
(b) contacting the glycoprotein product produced in step (a) with a reactant X capable of reacting with an aldehyde group, wherein reactant X comprises a linking moiety further comprising a second reactive, optionally protected, group to create a conjugate of the glycoprotein and the linking moiety; and
(c) contacting the product of step (b) with a protractor group capable of reacting with the second reactive group of the linking moiety to create a conjugate represented by the formula (glycoprotein)-(linking moiety)-(protractor group).

In a futher aspect, the present invention provides a pharmaceutical composition comprising a glycoprotein conjugate according to the invention.

### DESCRIPTION OF FIGURES

FIG. 1 shows the amount of exposed galactose residues on FVIIa after neuraminidase treatment (Vibro cholerae and Clostridium perfringens on agarose), as determined by galactose oxidase/amplex red assay from Molecular Probes (A-22179).

FIG. 2 shows an IEF-Gel analysis of FVIIa and FVIIa treated with neuramidase to produce asialo-FVIIa.

FIG. 3 shows MALDI-TOF spectra of FVIIa and asialo-FVIIa.

FIG. 4 shows an IEF-gel analysis (Invitrogen procedure, pH 3-7) with the following setup: lane 1: pI standard, lane 2: CP FVIIa−in MES−buffer, lane 3: CP FVIIa+agarose bound neuraminidase Vibro cholerae, 16 h, rt., lane 4: CP FVIIa+agarose bound neuraminidase Vibro Cholerae, 36 h, rt, lane 5: Asialo CP FVIIa+Galactose oxidase+aminoxyacetic acid, lane 6: CP FVIIa−in MES−buffer, lane 7: CP FVIIa+agarose bound neuraminidase−Clostridium perfringens, 16 h, rt, lane 8: CP FVIIa+agarose bound neuraminidase−Clostridium perfringens, 36 h, rt, lane 9: pI standard, lane 10: CP FVIIa−in MES−buffer.

FIG. 5 shows a SDS-PAGE gel (Invitrogen procedure, non-reducing conditions) with asialo FVIIa, and two fractions of PEG-5000 derivatized FVIIa obtained after galactose oxidase—catalase and subsequent reaction with PEG-5000 derivatized hydroxylamine.

FIG. 6 shows an IEF-gel analysis (Invitrogen procedure, pH 3-7) with the following setup: lane 1, pI standard; lane 2: FVIIa; lane 3: asialo FVIIa; lane 4-12 fractions of aminoxyacetic acid derivatized FVIIa after HiTrap ion-exchange purification.

FIG. 7 shows a MALDI-TOF spectrum of derivatized N-glycanes released by PNGase F treatment of FVIIa, which has been derivatized according to the process with nitrobenzyloxyamine.

FIG. 8 shows an IEF gel analysis (Invitrogen procedure, pH 3-7) with the following setup: lane 1: pI standard, lane 2: FVIIa, lane 3: FVIIa afterbuffer exchange to MES buffer, lane 4, asialo FVIIa (Vibro cholerae treatment); lane 5: blank; lane 6: galactose oxidase/catalase/aminoxyacetic acid treatment performed with 30 mU galactose oxidase; lane 7: galactose oxidase/catalase/aminoxyacetic acid treatment performed with 300 mU galactose oxidase.

FIG. 9 shows the amino acid sequence of wild-type human blood coagulation factor VII.

FIG. 10: illustrates the convergent solution phase synthesis of a first generation dendrimer capped with 2-(2-[2-methoxyethoxy]ethoxy)acetic acid.

FIG. 11: illustrates the solution phase synthesis of a second generation capped dendrimer with t-butyl protected carboxylic acid at the focal point.

FIG. 12: Illustrates the solid phase synthesis of a second generation dendrimer.

FIG. 13: illustrates the divergent solution phase synthesis of a second generation dendrimer, with free amino terminals and t-butyl protected carboxylic acid at the focal point.

FIG. 14: illustrates the solution phase end capping of a second generation dendrimer f.ex made as illustrated in FIGS. 12 og 13.

FIG. 15: illustrates the end capping of a second generation dendrimer using succinic acid mono tert-butyl ester and subsequent acid mediated deprotection to create a poly anionic glyco mimic polymer.

FIG. 16: illustrates solution phase functionalization of a second generation dendrimer, in order to create a handle compatible with aldehyde functionalities as obtainable by the invention.

FIG. 17: SDS-PAGE electrophoresis gel (non-denaturating). Lane 1: Mark-12 Mw standard (Invitrogen); lane 2: rFVIIa, lane 3: asialo rFVIIa, lane: 4-10 fractions containing modified rFVIIa with bands at 50 KDa+several 1.7 KDa band additions.

### DEFINITIONS

Natural peptides, obtained from eukaryote expression systems such as mammalian, insect or yeast cells, are frequently isolated in their glycosylated forms. The glycosyl moiety, also called the glycan moiety on such peptide, are themselves polyalcohols which either directly can be used for conjugation purposes, or by appropriate conditions can be converted into suitable attachment moieties for conjugation. The glycans of interest are either O-linked glycanes, i.e. glycoproteins where the glycan is linked via the amino acids residues serine or threonine; or N-glycans where the glycan moiety is linked to asparagine residues of the peptide.

The term “glycoprotein” is intended to encompass peptides, oligopeptides and polypeptides containing one or more sugar residues (glycans) attached to one or more amino acid residues of the “back bone” amino acid sequence. The glycans may be N-linked or O-linked.

As used herein, the term “glycan” or, interchangeable, “oligosaccharide chain” refers to the entire oligosaccharide structure that is covalently linked to a single amino acid residue. Glycans are normally N-linked or O-linked, e.g., glycans are linked to an asparagine residue (N-linked glycosylation) or a serine or threonine residue (O-linked glycosylation). N-linked oligosaccharide chains may be multi-antennary, such as, e.g., bi-, tri, or tetra-antennary and most often contain a core structure of GlcNAc-GlcNAc-Man₃.

Some glycoproteins, when produced in a human in situ, have a glycan structure with terminal, or “capping”, sialic acid residues, i.e., the terminal sugar of each antenna is N-acetylneuraminic acid linked to galactose via an α2->3 or α2->6 linkage. Other glycoproteins have glycans end-capped with other sugar residues. When produced in other circumstances, however, glycoproteins may contain oligosaccharide chains having different terminal structures on one or more of their antennae, such as, e.g., lacking sialic acid residues; containing N-glycolylneuraminic acid (Neu5Gc) residues; containing a terminal N-acetylgalactosamine (GalNAc) residue in place of galactose; and the like. Patterns of N-linked and/or O-linked oligosaccharides may be determined using any method known in the art, including, without limitation: high-performance liquid chromatography (HPLC); capillary electrophoresis (CE); nuclear magnetic resonance (NMR); mass spectrometry (MS) using ionization techniques such as fast-atom bombardment, electrospray, or matrix-assisted laser desorption (MALDI); gas chromatography (GC); and treatment with exoglycosidases in conjunction with anion-exchange (AIE)-HPLC, size-exclusion chromatography (SEC), mass spectroscopy (MS), gel electrophoresis (SDS-PAGE, CE-PAGE), isoelectric focusing gels, or iso-electric focusing capillary electrophoresis (CE-IEF) See, e.g., Weber et al., Anal. Biochem. 225:135 (1995); Klausen et al., J. Chromatog. 718:195 (1995); Morris et al., in Mass Spectrometry of Biological Materials, McEwen et al., eds., Marcel Dekker, (1990), pp 137-167; Conboy et al., Biol. Mass Spectrom. 21:397, 1992; Hellerqvist, Meth. Enzymol. 193:554 (1990); Sutton et al., Anal. Biohcem. 318:34 (1994); Harvey et al., Organic Mass Spectrometry 29:752 (1994).

The term “terminal sialic acid” or, interchangeable, “terminal neuraminic acid” is thus intended to encompass sialic acid residues linked as the terminal sugar residue in a glycan, or oligosaccharide chain, i.e., the terminal sugar of each antenna is N-acetyineuraminic acid linked to galactose via an α2->3 or α2->6 linkage.

The term “galactose or derivative thereof” means a galactose residue, such as natural D-galactose or a derivative thereof, such as an N-acetylgalactosamine residue. In one embodiment, the galactose derivative is N-acetylgalactosamine.

The term “terminal galactose or derivative thereof” means the galactose or derivative thereof linked as the terminal sugar residue in a glycan, or oligosaccharide chain, e.g., the terminal sugar of each antenna is galactose or N-acetylgalactosamine.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

The term “oxidized asialo glycoprotein” is intended to encompass asialo glycoproteins that have been oxidized by galactose oxidase, optionally in combination with a hydrogen peroxide scavenger such as, e.g., catalase or horseradish peroxidase, thus creating a reactive aldehyde functionality or moiety located on the terminal galactose residue.

The term “hydrogen peroxide scavenger” is intended to include compounds or substances able to react with, absorb, or neutralise hydrogen peroxide.

The term “reactant X capable of reacting with an aldehyde group” means a nucleophilic agents or reagents as well as other agents capable of reacting with an aldehyde group located in the oxidized terminal galactose residue of the glycoprotein, thus creating a covalent bond (or bonds) between the terminal galactose residue and the reactant (X). The reactant may in some embodiments of the invention include a polymeric group. Non-limiting examples of reactants X are hydroxylamines, O-alkylated hydroxylamines, amines, stabilized carbanions, stabilized enolates, hydrazides, alkyl hydrazides, hydrazines, acyl hydrazines, as well as ring forming (e.g. thiazolidine forming) nucleophiles such as, thioethanamines, cystein or cystein derivatives.

The term “protractor group” as used herein means a group which upon conjugation to a protein or peptide increase the circulation half-life of said protein or peptide, when compared to the un-modified protein or peptide. The specific principle behind the protractive effect may be caused by increased size, shielding of peptide sequences that can be recognized by peptidases or antibodies, or masking of glycanes in such way that they are not recognized by glycan specific receptores present in e.g. the liver or on macrophages, preventing or decreasing clearance. The protractive effect of the protractor group can e.g. also be caused by binding to blood components sush as albumin, or unspecific adhesion to vascular tissue. The conjugated glycoprotein should substantially preserve its biological activity.

The terms “electron withdrawing group” or “electron withdrawing groups” are used as defined in March, Advanced Organic Chemistry, 3rd edition, John Wiley & Sons, N.Y. 1985.

In one embodiment of the invention the protractor group is selected from the group consisting of:
(a) A low molecular organic charged radical (15-1000 Da), which may contain one or more carboxylic acids, amines sulfonic acids, phosphonic acids, or combination thereoff.
(b) A low molecular (15-1000 Da) neutral hydrophilic molecule, such as cyclodextrin, or a polyethylene chain which may optionally branched.
(c) A low molecular (15-1000 Da) hydrophobic molecule such as a fatty acid or cholic acid or derivatives theroff.
(d) Polyethyleneglycol with a avarage molecular weight of 2-40 KDa
(e) A well defined precission polymer such as a dendrimer with an excact molecular mass ranging from 700 to 20.000 Da, or more preferably between 700-10.000 Da.
(f) A substantially non imunogenic polypeptide such as albumin or an antibody or part of an antibody optionally containing a Fc-domain.
(g) A high molecular weight organic polymer such as dextran.

The term “nucleophilic agent” is interchangeable with “nucleophilic reagent” and “nucleophilic conjugation agent”.

As used in the present context, the term “covalent attachment” is meant to encompass that the oligosaccharide moiety (glycan) and the reactant X is either directly covalently joined to one another, or else is indirectly covalently joined to one another through an intervening moiety or moieties, such as a bridge, spacer, or linkage moiety or moieties.

The term “glycoconjugate” or, interchangeably, “conjugate” or “conjugate protein”, is intended to indicate a heterogeneous (in the sense of composite or chimeric) molecule formed by the covalent attachment of one or more terminal galactose residue(s) of a glycoprotein to one or more nucleophilic agents (reactants X). In the present context the term “protein” is intended to include peptides, oligopeptides, and polypeptides having sequences of at least 4 amino acid residues, preferably having between 4 and about 1000 residues.

In one embodiment of the invention the protractor group is a polymeric molecule.

As used herein the term “polymeric molecule” means a molecules formed by covalent linkage of two or more monomers wherein none of the monomers is an amino acid residue.

In one embodiment of the invention the polymeric molecule is selected from the group consisting of dendrimers, polyalkylene oxide (PAO), including polyalkylene glycol (PAG), such as polyethylene glycol (PEG) and polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, and dextran, including carboxymethyl-dextran. In one embodiment of the invention, the polymeric molecule is a PEG group. In one embodiment of the invention, the polymeric molecule is a dendrimer.

In one embodiment of the invention, the polymeric molecule is a dendrimer with a molecular weight in the range of 700-10.000 Da

The polymer molecule is part of, or is attached to, a Reactant X capable of reacting with an aldehyde group in the oxidized galactose residue located in the glycopeptide.

A group present on the polymer may be activated to create a reactant X, as described above, before reaction with the oligosaccharide moiety or glycan. The activated group, whether present on the oligosaccharide- or polymer moiety may be in the form of an activated leaving group. Methods and chemistry for activation of polymers are described in the literature. Commonly used methods for activation of polymers include activation of functional groups with cyanogen bromide, periodate, glutaraldehyde, biepoxides, epichlorohydrin, divinylsulfone, carbodiimide, sulfonyl halides, trichlorotriazine, etc. (see, for example, Taylor (1991). Such electrophilic activated polymers can then be converted to reagents of type reagent X by reaction with e.g. binucleophilic linker moieties. Protein Immobilization, Fundamentals and Applications, Marcel Dekker, N.Y.; Wong (1992), Chemistry of protein Conjugation and Crosslinking, CRC Press, Boca Raton; Hermanson et al., (1993), Immobilized Affinity Ligand Techniques, Academic Press, N.Y.; Dunn et al., Eds. Polymeric Drugs and Drug Delivery Systems, ACS Symposium Series Vol. 469, American Chemical Society, 1991.)

The term “branched polymer”, or interchangebly “dendritic polymer”, “dendrimer” or “dendritic structure” means an organic polymer assembled from a selection of monomer building blocks of which, some contains branches.

The term “generation” means a single uniform layer, created by reacting one or more identical functional groups on an organic molecule with a particular monomer building block. With a branched polymer made from exclusively bifurcated monomers, the number of reactive groups in a generation is given by the formula (2*(m−1))2, where m is an integer of 1, 2, 3 . . . 8 representing the particular generation. For a branched polymer made from exclu-sively trifurcated monomers, the number of reactive groups is given by the formula (3*(m−1))3, and for a branched polymer made exclusively from a multifurcated monomer with n-branches, the number of reactive groups is given by (n*(m−1))n. For branched polymers in which different monomers are used in each individual generation, the number of reactive groups in a particular layer or generation can be calculated recursively knowing the layer po-sition and the number of branches of each individual monomers in the dendritic structure.

In one embodiment of the invention the protractor group is a selected from the goup consisting of serum protein binding-ligands, such as compounds which bind to albumin, like fatty acids, C5-C24 fatty acid, aliphatic diacid (e.g. C5-C24). Other examples of protractor groups includes small organic molecules containing moieties that under physiological conditions alters charge properties, such as carboxylic acids or amines, or neutral substituents that prevent glycan specific recognition such as smaller alkyl substituents (e.g., C1-C5 alkyl). In one embodiment of the invention the protractor group is albumin. In one embodiment of the invention the protractor group is selected from the group consisting of: dendrimer, polyalkylene oxide (PAO), polyalkylene glycol (PAG), polyethylene glycol (PEG), polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, dextran, carboxymethyl-dextran; serum protein binding-ligands, such as compounds which bind to albumin, such as fatty acids, C5-C24 fatty acid, aliphatic diacid (e.g. C5-C24), a structure (e.g. sialic acid derivatives or mimetics) which inhibits the glycans from binding to receptors (e.g. asialoglycoprotein receptor and mannose receptor), a small organic molecule containing moieties that under physiological conditions alters charge properties, such as carboxylic acids or amines, or neutral substituents that prevent glycan specific recognition such as smaller alkyl substituents (e.g., C1-C5 alkyl), a low molecular organic charged radical (e.g. C1-C25), which may contain one or more carboxylic acids, amines sulfonic, phosphonic acids, or combination thereof; a low molecular neutral hydrophilic molecule (e.g. C1-C25), such as cyclodextrin, or a polyethylene chain which may optionally branched; polyethyleneglycol with a avarage molecular weight of 2-40 KDa; a well defined precission polymer such as a dendrimer with an excact molecular mass ranging from 700 to 20.000 Da, or more preferably between 700-10.000 Da; and a substantially non-imunogenic polypeptide such as albumin or an antibody or part of an antibody optionally containing a Fc-domain.

The term “activated leaving group” includes those moieties which are easily displaced in organic- or enzyme-regulated substitution reactions. Activated leaving groups are known in the art, see, for example, Vocadlo et al., In Carbohydrate Chemistry and Biology, Vol 2, Wiley-VCH Verlag, Germany (2000); Kodama et al., Tetrahedron Letters 34:6419 (1993); Lougheed et al., J. Biol. Chem. 274:37717 (1999).

Reactive groups and classes of reactions useful in practising the present invention are generally those which proceed under relatively mild conditions. These include, but are not limited to nucleophilic substitutions (e.g., reaction of amines and alcohols with acyl halides, active esters). These and other useful reactions are described in, for example, March, Advanced Organic Chemistry, 3rd edition, John Wiley & Sons, N.Y. 1985; Hermanson, Bioconjugate Techniques, Academic Press, San Diego, 1996; Feeney et al, Modifications of Proteins, Advances in Chemistry Series, Vol. 198, American Chemical Society, 1982.

The reactive functional groups can be selected such that they do not participate in, or interfere with, the reactions necessary to assemble the oligosaccharide and the polymer moiety. Alternatively, a reactive functional group can be protected from participating in the reaction by the presence of a protective group. For examples of useful protecting groups, see, for example, Greene et al., Protective groups in Organic Synthesis, John Wiley & Sons, N.Y., 1991.

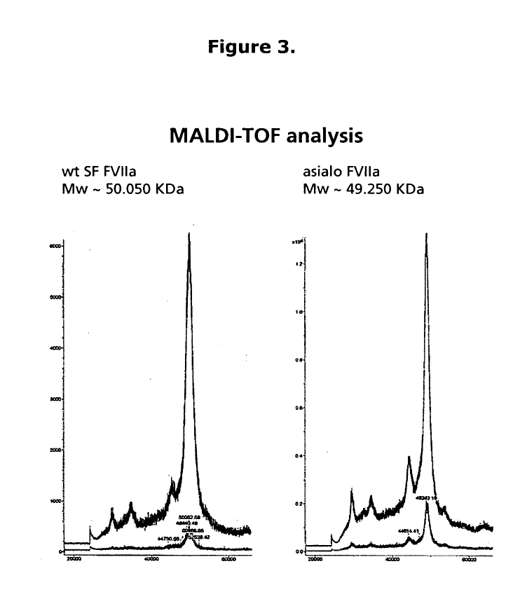

The term “naturally occurring glycosylation site in FVII” is intended to indicate the glycosylation sites at positions Asn-145 (N145), Asn-322 (N322), Ser-52 (S52), and Ser-60 (S60) of the amino acid sequence as shown in FIG. 3. In a similar way, the term “naturally occurring in vivo O-glycosylation site” includes the positions S52 and S60, whereas the term “naturally occurring in vivo N-glycosylation site” includes the positions N145 and N322. The term “amino acid residues corresponding to amino acid residues S52, S60, N145, N322 of FIG: 3 (FVII wt.)” is intended to indicate the Asn and Ser amino acid residues corresponding to the sequence of wild-type Factor VII (FIG. 3) when the sequences are aligned. Amino acid sequence homology/identity is conveniently determined from aligned sequences, using a suitable computer program for sequence alignment, such as, e.g., the ClustalW program, version 1.8, 1999 (Thompson et al., 1994, Nucleic Acid Research, 22: 4673-4680).

The term “functional in vivo half-life” is used in its normal meaning, i.e., the time at which 50% of the biological activity of the polypeptide or conjugate is still present in the body/target organ, or the time at which the activity of the polypeptide or conjugate is 50% of its initial value. As an alternative to determining functional in vivo half-life, “in vivo plasma half-life” may be determined, i.e., the time at which 50% of the polypeptide or conjugate molecules circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life. The functionality to be retained is normally selected from procoagulant, proteolytic, co-factor binding, receptor binding activity, or other type of biological activity associated with the particular protein.

The term “increased” as used about the functional in vivo half-life or plasma half-life is used to indicate that the relevant half-life of the polypeptide or conjugate is statistically significantly increased relative to that of a reference molecule, such as non-conjugated glycoprotein as determined under comparable conditions. For instance the relevant half-life may be increased by at least about 25%, such as by at lest about 50%, e.g., by at least about 100%, 150%, 200%, 250%, or 500%. In some embodiments, the preparations of the present invention exhibit an increase in half-life of at least about 0.25 h, preferably at least about 0.5 h, more preferably at least about 1 h, and most preferably at least about 2 h, relative to the half-life of a reference preparation.

“Immunogenicity” of a preparation refers to the ability of the preparation, when administered to a human, to elicit a deleterious immune response, whether humoral, cellular, or both. In any human sub-population, there may exist individuals who exhibit sensitivity to particular administered proteins. Immunogenicity may be measured by quantifying the presence of anti-glycoprotein antibodies and/or glycoprotein responsive T-cells in a sensitive individual, using conventional methods known in the art. In some embodiments, the preparations of the present invention exhibit a decrease in immunogenicity in a sensitive individual of at least about 10%, preferably at least about 25%, more preferably at least about 40% and most preferably at least about 50%, relative to the immunogenicity for that individual of a reference preparation.

By “linker moiety” or “L1” is meant any biocompatible molecule functioning as a means of linking a protractor group to “reactant X”, which is capable of reacting with an aldehyde group. It is to be understood that in the final conjugate of a glycoprotein the “linker moiety” is linking via chemical bonds the glycoprotein to the protractor group. It is to be understood, that the linker moiety may contain both covalent and non-covalent chemical bonds or mixtures thereof.

Suitable linker moieties comprise group(s) such as, but are not limited to, peptides; polynucleotides; sacharides including monosaccharides, di- and oligosaccharides, cyclodextrins and dextran; polymers including polyethylene glycol, polypropylene glycol, polyvinyl alcohol, hydrocarbons, polyacrylates and amino-, hydroxy-, thio- or carboxy-functionalised silicones, other biocompatible material units; and combinations thereof. Such linker moiety materials described above are widely commercially available or obtainable via synthetic organic methods commonly known to those skilled in the art.

The linker moiety may, for example, be selected among the following structures: straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl. The linker moiety may be straight chained or branched and may contain one or more double or triple bonds. The linker moiety may contain one or more heteroatoms like N, O or S. It is to be understood, that the linker moiety can comprise more than one class of the groups described above, as well as being able to comprise more than one member within a class. Where the linker moiety comprises more than one class of group, such linker moiety is preferably obtained by joining different units via their functional groups. Methods for forming such bonds involve standard organic synthesis and are well known to those of ordinary skill in the art.

The terms “alkyl” or “alkylene” refer to a C1 6 alkyl or -alkylene, representing a satu-rated, branched or straight hydrocarbon group having from 1 to 6 carbon atoms. Typical C1 6 alkyl groups include, but are not limited to, methyl, ethyl, n-propyl, isopropyl, butyl, isobu-tyl, sec-butyl, tert-butyl, pentyl, hexyl and the corresponding divalent radicals.

The terms “alkenyl” or “alkenylene” refer to a C2-6 alkenyl or -alkenylene, represent-ing a branched or straight hydrocarbon group having from 2 to 6 carbon atoms and at least one double bond. Typical C2 6 alkenyl groups include, but are not limited to, ethenyl, 1-propenyl, 2-propenyl, isopropenyl, 1,3 butadienyl, 1-butenyl, 2-butenyl, 1-pentenyl, 2-pentenyl, 1-hexenyl, 2-hexenyl, 1-ethylprop-2-enyl, 1,1-(dimethyl)prop-2-enyl, 1-ethylbut-3-enyl, 1,1-(dimethyl)but-2-enyl, and the corresponding divalent radicals.

The terms “alkynyl” or “alkynylene” refer to a C2 6 alkynyl or -alkynylene, represent-ing a branched or straight hydrocarbon group having from 2 to 6 carbon atoms and at least one triple bond. Typical C2 6 alkynyl groups include, but are not limited to, vinyl, 1-propynyl, 2-propynyl, isopropynyl, 1,3 butadynyl, 1-butynyl, 2-butynyl, 1-pentynyl, 2-pentynyl, 1-hexynyl, 2-hexynyl, 1-ethylprop-2-ynyl, 1,1-(dimethyl)prop-2-ynyl, 1-ethylbut-3-ynyl, 1,1-(dimethyl)but-2-ynyl, and the corresponding divalent radicals.

The terms “alkyleneoxy” or “alkoxy” refer to “C1-6-alkoxy” or -alkyleneoxy representing the radical —O—C1-6-alkyl or —O—C1-6-alkylene, wherein C1 6 alkyl(ene) is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “heteroaryl” as used herein is intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

### DETAILED DESCRIPTION OF THE INVENTION

It is an object of this invention to provide means for stabilizing glycoproteins in circulation both when the glycosylation of that protein provides the primary determinant for clearance, and when other clearance pathways not related to the glycan moiety are operational.

In the former case, increased half-life is achieved in such glycoproteins by treatments which block or inhibit removal of the protein by sugar-specific receptor, such as the galactose and mannose receptors in the liver. In particular, the prolonging of soluble glycoprotein derivatives in circulation is described. In the later case, increased half-life can be achieved by for example attaching a “protractor” moiety such as, e.g., a serum protein binding ligand, such as albumin binding ligands, or a large polymer such as PEG. In both cases, prolonged circulating half-lives are desirable in therapeutic proteins because frequency and/or size of dose can be reduced when half-life is longer.

Galactose oxidase (GO), is a copper enzyme that catalyzes the two-electron oxidation of a large number of primary alcohols to their corresponding aldehydes, coupled with the reduction of dioxygen to hydrogen peroxide: RCH₂OH+O₂→RCHO+H₂O₂. The enzyme exhibits a broad specificity for reductant, and a wide variety of primary alcohols serve as effective substrates, including substituted benzyl alcohols. Galactose oxidase together with catalase or horseradish peroxidase offers a mild and highly selective method for introducing aldehyde functionalities into glycoproteins, and has been extensively used for either radio or fluorescence labelling of purified glycoproteins, as well as glycoproteins embedded in lipid membranes of full cellular systems (see for example Baumann H. and Doyle D., J. Biol. Chem. 1979, 254 (7) 2542-2550 and Wilchek M.; Spiegel S. and Spiegel Y., Biochem. Biophys Res. Commun, 1980, 92(4) 1215-1222). Galactose oxidase selectively oxidizes galactose (Gal) and acetylated galactosamine (GalNAc).

Neuraminidase and galactose oxidase both have been immobilized on beads in order to produce cost efficient high storage and operational stable enzyme reactors, see for example Bilkova Z. et. al., J. Chromatography B. 2002, 770/1-2 (177-181). The application of such immobilized enzyme, optionally in connection with similar immobilized enzyme like catalase of horseradish peroxidase for the removal of hydrogen peroxide side product, are within the scope of the invention. Beside increased stability immobilized enzymes offers advantages in subsequent purification processes because they are easily removed by simple filtration. Thus in one embodiment of the invention, one or more of the enzymes involved in the process are immobilized on support.

Sialidase Treatment

Some glycoproteins contain terminal sialic acid residues. In such cases, the glycoprotein, which either can be obtained from natural sources, or from genetically modified mammalian cells such as COS-, BHK- or CHO cells, or from remodelled proteins obtained from yeast cells, is initially treated with sialidase, in order to expose at least one galactose residue from the inner layer of galactose residues. Some glycoproteins are by nature not end-sialylated (does not contain terminal sialic acid residues), and sialidase treatment is in such cases obsolete. Enzymatic de-sialylation can be performed either by use of soluble sialidases of commercial origin, by use of recombinant genetically modified sialidases or by use of sialidases bound to solid supports such as, e.g., agarose. The reaction progress can conveniently be monitored by IEF-gel monitoration as described in WO 2003031464 (Neose), or by capillary electrophoresis techniques such as those described in N. K. Klausen and T. Kornfelt, J. Chromatography A, 718, 195-202 (1995). If soluble sialidases are used for the reaction, a purification step can be necessary. Suitable purification techniques are known to the skilled person, and can involve, e.g., ion-exchange chromatography, or other similar technique.

Galactose Oxidase

The glycoprotein, or the asialo-glycoprotein obtained by treatment with sialidase, is subsequently treated with galactose oxidase using molecular oxygen as oxidant. As the by-product of the reaction is hydrogen peroxide, some measure to avoid its destructive power (e.g. oxidation of methionine residues, etc.) is generally taken, for example by adding a hydrogen peroxide scavenger. Both catalase and horseradish peroxidase are suitable, but other scavengers are known to the skilled person. As the overall change in structure and function of the glycoprotein is minute in this reaction step, the reaction is more difficult to monitor. Chemical conjugation with reporter molecules such as dansyl hydrazid is an option. Alternatively, if horseradish peroxidase or catalase is omitted, the reaction progress can be colorimetricly estimated using a commercially available kit, such as Amplex Red from Molecular Probes.

Chemically Conjugation Step

In one embodiment of the invention the glycoprotein for the chemical conjugation step is a glycoprotein which has been treated with sialidase to remove sufficient sialic acid to expose at least one galactose residue and which has been further treated, e.g., with galactose oxidase and horseradish peroxidase to produce a free reactive aldehyde functionality.

One reaction sequence is depicted below, using a reactant X capable of reacting with an aldehyde group: 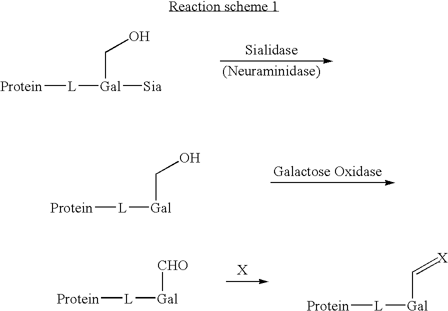
where Sia denotes a sialic acid linked to a galactose or galactose derivative (Gal) in either alpha-2,3-, or alpha-2,6-configuration.
In one embodiment the Gal-OH represent galactose in which case,

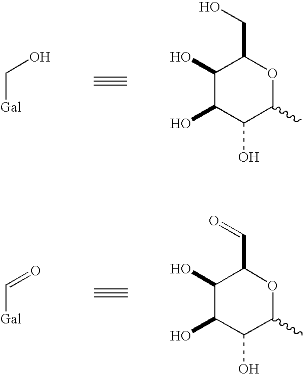

In one embodiment Gal-OH represent the galactose derivative N-acetyl galactosamine and the galactose oxidase oxidizes the acetylated galactosamine residues in which case,

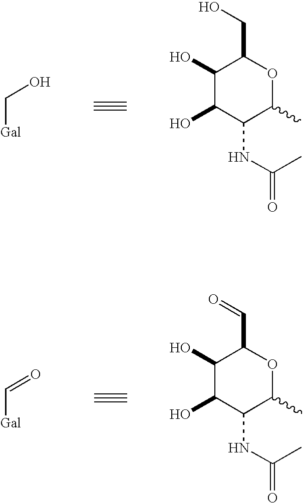

X is any type of molecule containing a chemical functionality that can react covalently with an aldehyde to form a C-6 modified galactose or N-acetyl galactosamine residue (such as, e.g., a nucleophile agent).

L is a divalent organic radical linker which may be any organic di-radical including those containing one or more carbohydrate moiety(-ies) consisting of natural monosaccharide(s), such as fucose, mannose, N-acetyl glycosamine, xylose, and arabinose, interlinked in any order and with any number of branches. L may also be a valence bond.

The chemical conjugation may be performed in a number of ways depending on the particular reactant X involved.

In one embodiment of the invention, reactant X has the formula nuc-R, wherein nuc is a functional group which can react with an aldehyde to form a covalent bond, and R is a protractor group.

In one embodiment of the invention, reactant X has the formula nuc-L1-R, wherein nuc is a functional group which can react with an aldehyde to form a covalent bond, R is a protractor group, and L1 is a linking moiety.

In one embodiment of the invention, reactant X is selected from the group consisting of: H₂N—R, HR1N—R, H₂N—O—R, HR1N—O—R, H₂N—NH—CO—R, H₂N—CHR1—CHR—SH, H₂N—CHR—CHR1-SH, H₂N—NH—SO₂—R, and Z′-CH₂-Z″-R; wherein R is a protractor group; R1 is H or a second protractor group; Z′ and Z″ represent electron withdrawing groups, such as, e.g., COOEt, CN, NO₂, and wherein one or both of the Z groups can be connected to the R group.

In one embodiment of the invention, reactant X is selected from the group of: H₂N-L1-R, HR1N-L1-R, H₂N—O-L1-R, HR1N—O-L1-R, H₂N—NH—CO-L1-R, H₂N—CHR1-CH(L1-R)—SH, H₂N—CH(L1-R)—CHR1-SH, H₂N—NH—SO₂-L1-R, Z′-CH₂-Z″-L1-R; wherein L1 is a linking moiety, R is a protractor group, R1 is H or a second protractor group; Z′ and Z″ represent electron withdrawing groups, such as, e.g., COOEt, CN, NO₂, and wherein one or both of the Z groups can be connected to the R group.

In one embodiment of the invention, reactant X has the formula nuc-L1-Rₙ, wherein nuc is a functional group which can react with an aldehyde to form a covalent bond, and R is a protractor group, and L1 is a polyfunctional linking moiety connecting one or more protractor groups (R) to the reactive functionality (nuc), and wherein L1 may or may not contribute to the protraction; n represents an integer, n=1-25, such as 1-10.

In a specific embodiment reactant X is given by general formula:
nuc-R

where nuc is a group that can react with an aldehyde group. Non-limiting examples for illustration include hydroxylamines, O-alkylated hydroxylamines, amines, stabilized carbanions, stabilized enolates, hydrazides, alkyl hydrazides, hydrazines, acyl hydrazines etc. Other embodiments includes ring forming (e.g. thiazolidine forming) nucleophiles such as, e.g., thioethanamines, cystein or cystein derivatives, α-mercaptoacylhydrazides ect.

In particular nuc can be:

| | |
|---|---|
| hydrazine derivatives | —NH—NH₂, |
| hydrazine carboxylate | —O—C(O)—NH—NH₂, |
| derivatives | |
| semicarbazide derivatives | —NH—C(O)—NH—NH₂, |
| thiosemicarbazide | —NH—C(S)—NH—NH₂, |
| derivatives | |
| carbonic acid dihydrazide | —NHC(O)—NH—NH—C(O)—NH—NH₂, |
| derivatives | |
| carbazide derivatives | —NH—NH—C(O)—NH—NH₂, |
| thiocarbazide derivatives | —NH—NH—C(S)—NH—NH₂, |
| aryl hydrazine derivatives | —NH—C(O)—C₆H₄—NH—NH₂, |
| hydrazide derivatives | —C(O)—NH—NH₂, and |
| oxylamine derivatives, such | —O—NH₂, —C(O)—O—NH₂, |
| as | —NH—C(O)—O—NH₂ and |
| | —NH—C(S)—O—NH₂. |

R of general formula nuc-R may be an organic radical selected from one of the groups below:
a) straight, branched and/or cyclic C₁₋₃₀alkyl, C₂₋₃₀alkenyl, C₂₋₃₀alkynyl, C₁₋₃₀heteroalkyl, C₂₋₃₀heteroalkenyl, C₂₋₃₀heteroalkynyl, wherein one or more homocyclic aromatic compound biradical or heterocyclic compound biradical may be inserted, and wherein said C₁₋₃₀ or C₂₋₃₀ radicals may optionally be substituted with one or more substituents selected from —CO₂H, —SO₃H, —PO₂OH, —SO₂NH₂, —NH₂, —OH, —SH, halogen, or aryl, wherein said aryl is optionally substituted with —CO₂H, —SO₃H, —PO₂OH, —SO₂NH₂, —NH₂, —OH, —SH, or halogen; steroid radicals; lipid radicals;
b) polysaccharide radicals, e.g. dextrans; α-, β-, or γ-cyclodextrin, polyamide radicals e.g. polyamino acid radicals; PVP radicals; PVA radicals; poly(1-3-dioxalane); poly(1,3,6-trioxane); ethylene/maleic anhydride polymer;
c) Cibacron dye stuffs, such as Cibacron Blue 3GA, and polyamide chains of specified length, as disclosed in WO 00/12587, which is incorporated herein by reference.
d) a substantially non-immunogenic protein residue such as a blood component like albuminyl derivative, or a antibody or a domain thereoff such as a Fc domain from human normal IgG1, as described in Kan, S K et al in The Journal of Immunology 2001, 166(2), 1320-1326 or in Stevenson, G T, The Journal of Immunology 1997, 158, 2242-2250.
e) polyethylene glycol (PEG) or methoxy polyethylene glycol (mPEG) radicals and amino derivatives thereof, where the avarage molecular weight may be between 500 and 100,000 Da, such as between 500 and 60,000 Da, such as between 1000 and 40,000 Da, such as between 5000 and 40,000 Da.
f) R may also represent —C(R5)₃, wherein each R1 independently represents hydrogen or a moiety selected from amongst -D-((CH₂)_{q}O)ᵣ—OR6, -D-CH2-O—((CH₂)_{q}O)ᵣ—OR6, -D-O—((CH₂)_{q}O)ᵣ—OR6; wherein q represents 1-6, r represent 10 to 500, and R6 represent hydrogen or C₁-C₆-alkyl; and wherein D represents a bond or C₁₋₈alkyl or C₁₋₈ heteroalkyl;
g) moieties that are known to bind to plasma proteins, such as e.g. albumin, where the albumin binding property may be determined as described in J. Med. Chem, 43, 2000, 1986-1992, which is incorporated herein by reference, or an albumin binding moiety such as a peptide comprising less than 40 amino acid residues such as moieties disclosed in J. Biol. Chem. 277, 38 (2002) 35035-35043, which is incorporated herein by reference.
h) C₁-C₂₀-alkyl, such as C₁-C₁₈-alkyl. Specific mentioning is made of C₁₄-, C₁₆- and C₁₈-alkyl, which optionally may be substituted with in particular charged groups, polar groups and/or halogens. Examples of such substituents include —CO₂H and halogen. In a particular embodiment, all hydrogens in the C₁-C₂₀-alkyl are substituted with fluoro to form perfluoroalkyl.

### SPECIFIC EMBODIMENTS

Specific embodiments of R-nuc, which is PEG-2-40K derivatives is shown in the following by illustration and not limitation:

In one embodiment of the invention, the reactant X has the formula nuc-R, wherein nuc is a functional group which can react with an aldehyde to form a covalent bond, and R is a PEG group. In one embodiment of the invention, the nuc-R is selected from the group consisting of: 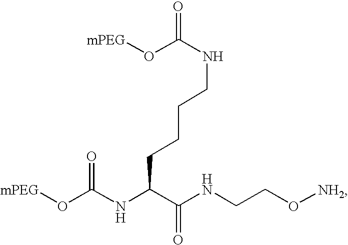
wherein mPEG has a molecular weight of 20 kDa, 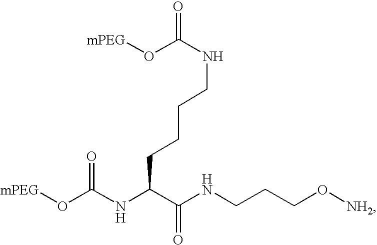
wherein mPEG has a molecular weight of 20 kDa, 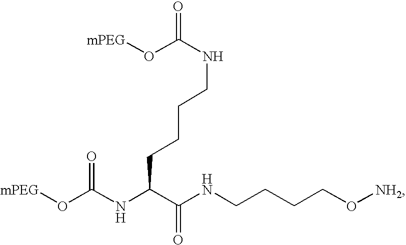
wherein mPEG has a molecular weight of 20 kDa, 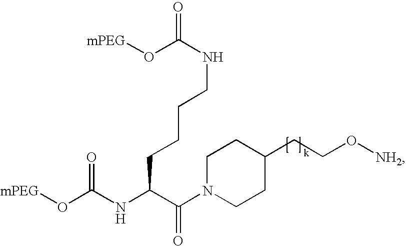
wherein mPEG has a molecular weight of 20 kDa, 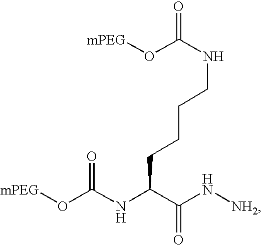
wherein mPEG has a molecular weight of 20 kDa, 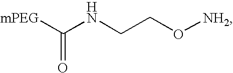
wherein mPEG has a molecular weight of 20 kDa, 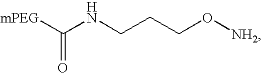
wherein mPEG has a molecular weight of 20 kDa, 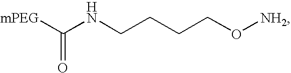
wherein mPEG has a molecular weight of 20 kDa, 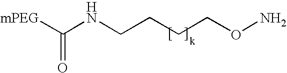
wherein mPEG has a molecular weight of 20 kDa, 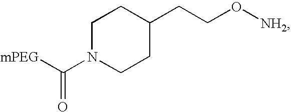
wherein mPEG has a molecular weight of 20 kDa, 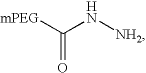
wherein mPEG has a molecular weight of 20 kDa, 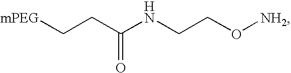
wherein mPEG has a molecular weight of 20 kDa, 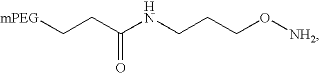
wherein mPEG has a molecular weight of 20 kDa, 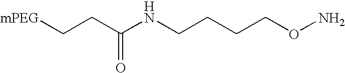
wherein mPEG has a molecular weight of 20 kDa, 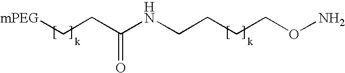
wherein mPEG has a molecular weight of 20 kDa, 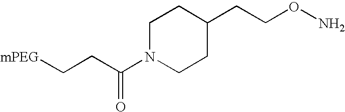
wherein mPEG has a molecular weight of 20 kDa, 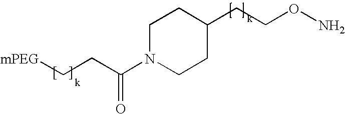
wherein mPEG has a molecular weight of 20 kDa, 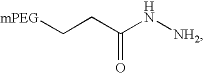
wherein mPEG has a molecular weight of 20 kDa, 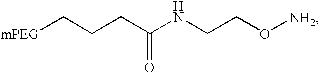
wherein mPEG has a molecular weight of 20 kDa, 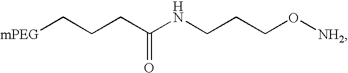
wherein mPEG has a molecular weight of 20 kDa, 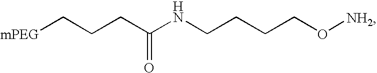
wherein mPEG has a molecular weight of 20 kDa, 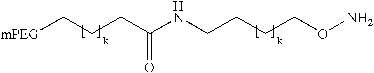
wherein mPEG has a molecular weight of 20 kDa, 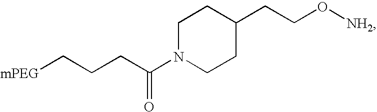
wherein mPEG has a molecular weight of 20 kDa, 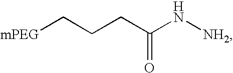
wherein mPEG has a molecular weight of 20 kDa, 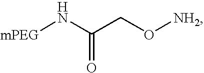
wherein mPEG has a molecular weight of 20 kDa, 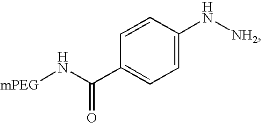
wherein mPEG has a molecular weight of 20 kDa, 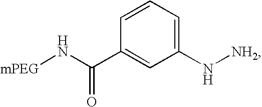
wherein mPEG has a molecular weight of 20 kDa, 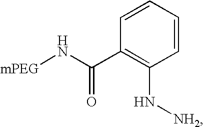
wherein mPEG has a molecular weight of 20 kDa, 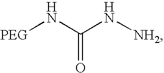
wherein mPEG has a molecular weight of 20 kDa, 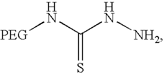
wherein mPEG has a molecular weight of 20 kDa, 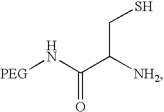
wherein mPEG has a molecular weight of 20 kDa. 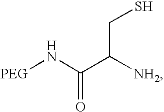
wherein mPEG has a molecular weight of 10 kDa. 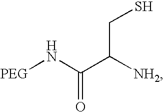
wherein mPEG has a molecular weight of 5 kDa. 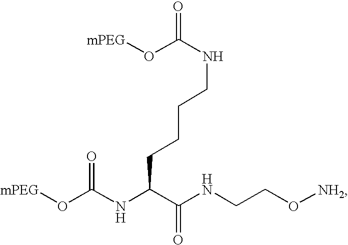
wherein mPEG has a molecular weight of 10 kDa, 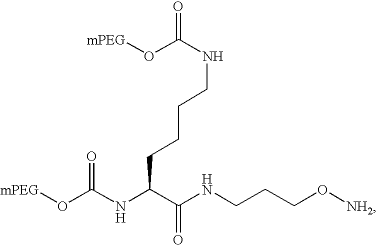
wherein mPEG has a molecular weight of 10 kDa, 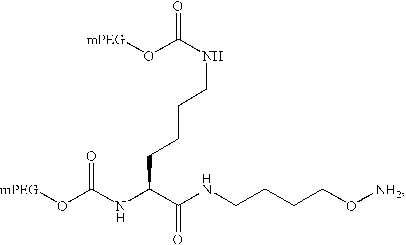
wherein mPEG has a molecular weight of 10 kDa, 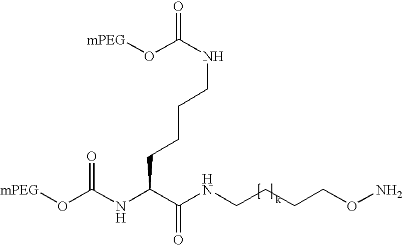
wherein mPEG has a molecular weight of 10 kDa, 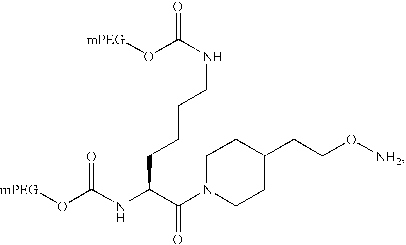
wherein mPEG has a molecular weight of 10 kDa, 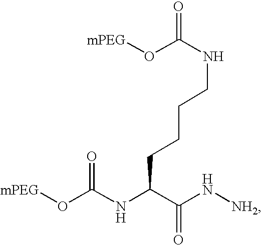
wherein mPEG has a molecular weight of 10 kDa, 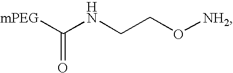
wherein mPEG has a molecular weight of 10 kDa, 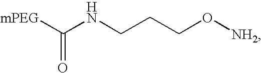
wherein mPEG has a molecular weight of 10 kDa, 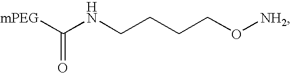
wherein mPEG has a molecular weight of 10 kDa, 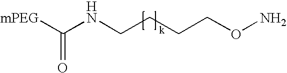
wherein mPEG has a molecular weight of 10 kDa, 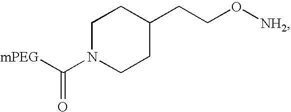
wherein mPEG has a molecular weight of 10 kDa, 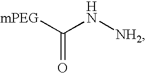
wherein mPEG has a molecular weight of 10 kDa, 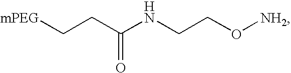
wherein mPEG has a molecular weight of 10 kDa, 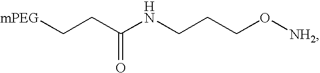
wherein mPEG has a molecular weight of 10 kDa, 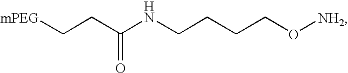
wherein mPEG has a molecular weight of 10 kDa, 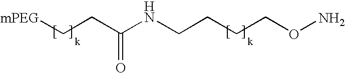
wherein mPEG has a molecular weight of 10 kDa, 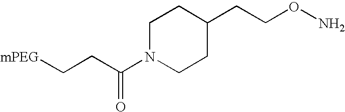
wherein mPEG has a molecular weight of 10 kDa, 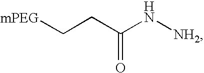
wherein mPEG has a molecular weight of 10 kDa, 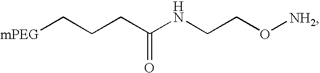
wherein mPEG has a molecular weight of 10 kDa, 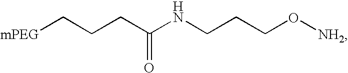
wherein mPEG has a molecular weight of 10 kDa, 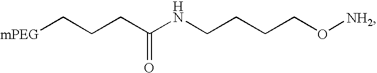
wherein mPEG has a molecular weight of 10 kDa, 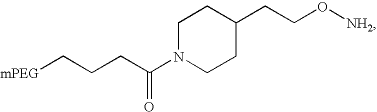
wherein mPEG has a molecular weight of 10 kDa, 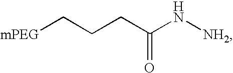
wherein mPEG has a molecular weight of 10 kDa, 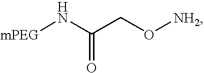
wherein mPEG has a molecular weight of 10 kDa, 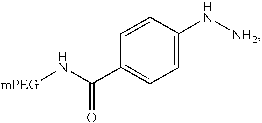
wherein mPEG has a molecular weight of 10 kDa, 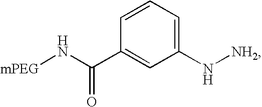
wherein mPEG has a molecular weight of 10 kDa, 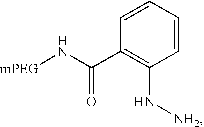
wherein mPEG has a molecular weight of 10 kDa, 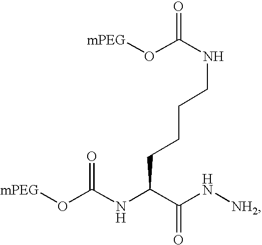
wherein mPEG has a molecular weight of 10 kDa, 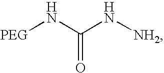
wherein mPEG has a molecular weight of 10 kDa, 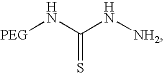
wherein mPEG has a molecular weight of 10 kDa,

In one embodiment of the invention, the protractor group is a compound which bind to albumin.

The phrase “compound which bind to albumin” is used herein interchangeable with “albumin binder”.

In one embodiment of the invention, the reactant X has the formula nuc-R, wherein nuc is a functional group which can react with an aldehyde to form a covalent bond, and R is a compound which bind to albumin. In one embodiment of the invention, the nuc-R is selected from the group consisting of 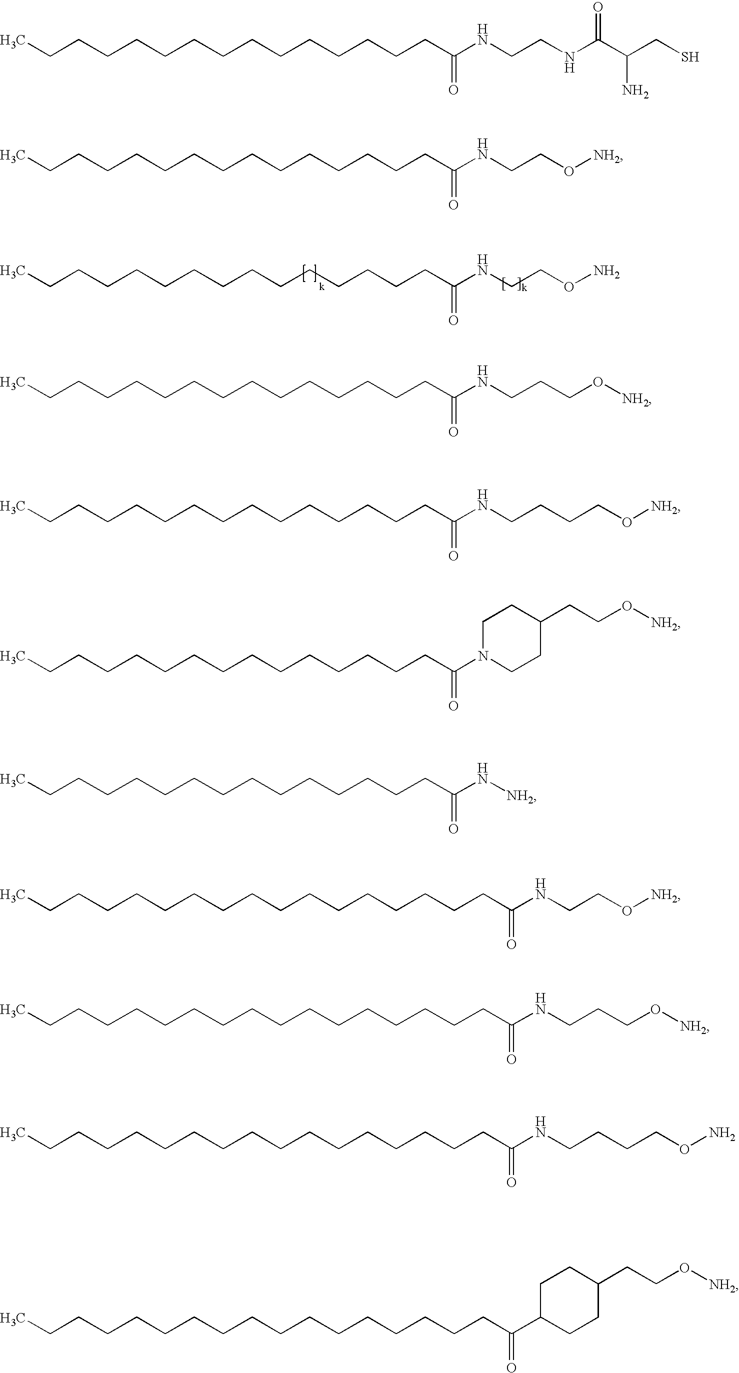 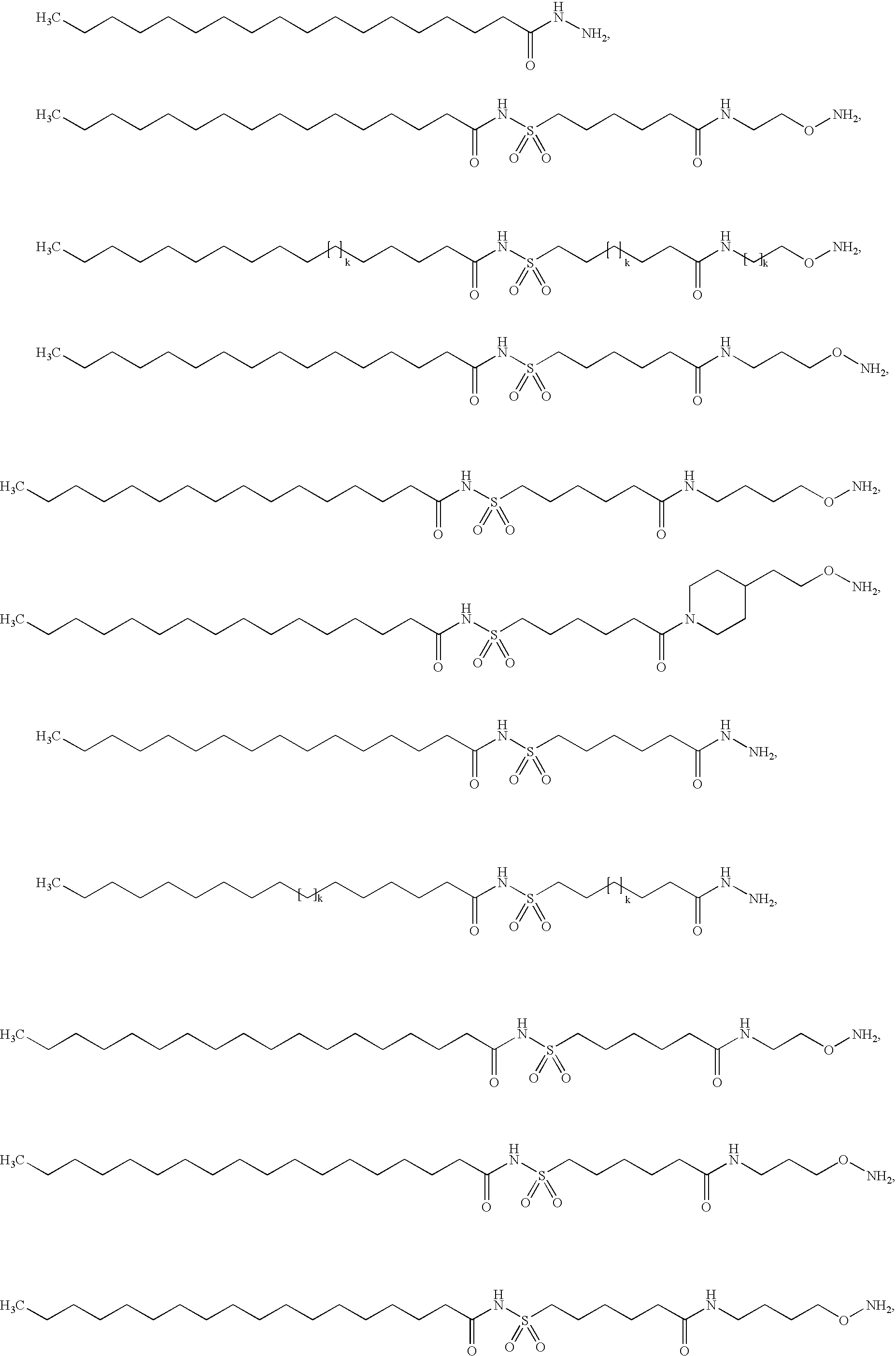 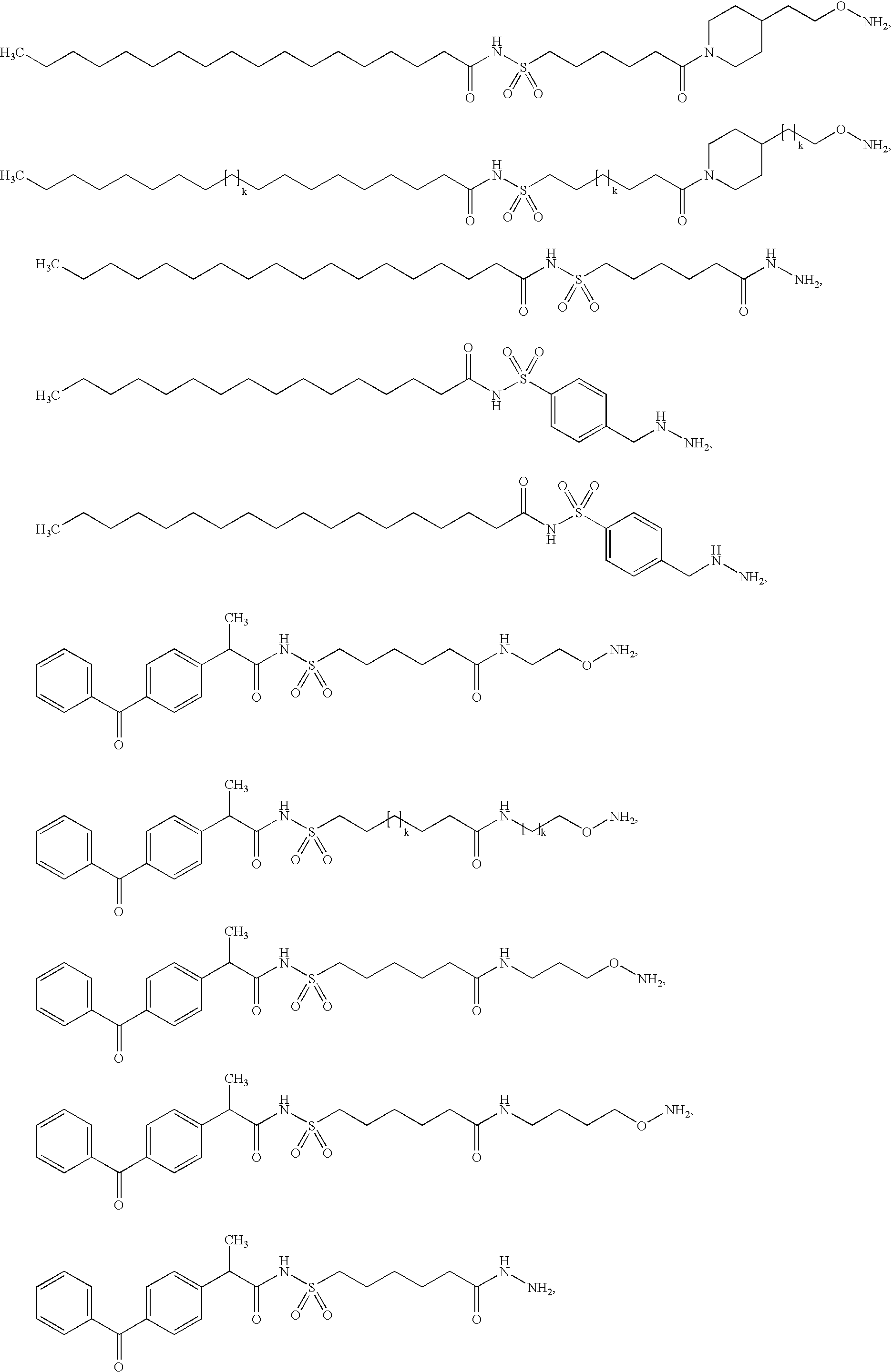
where k is an integer of between 0 and 10, such as 1, 2, 3, 4, 5, 6, 7, 8, 9, or 10. and the structures

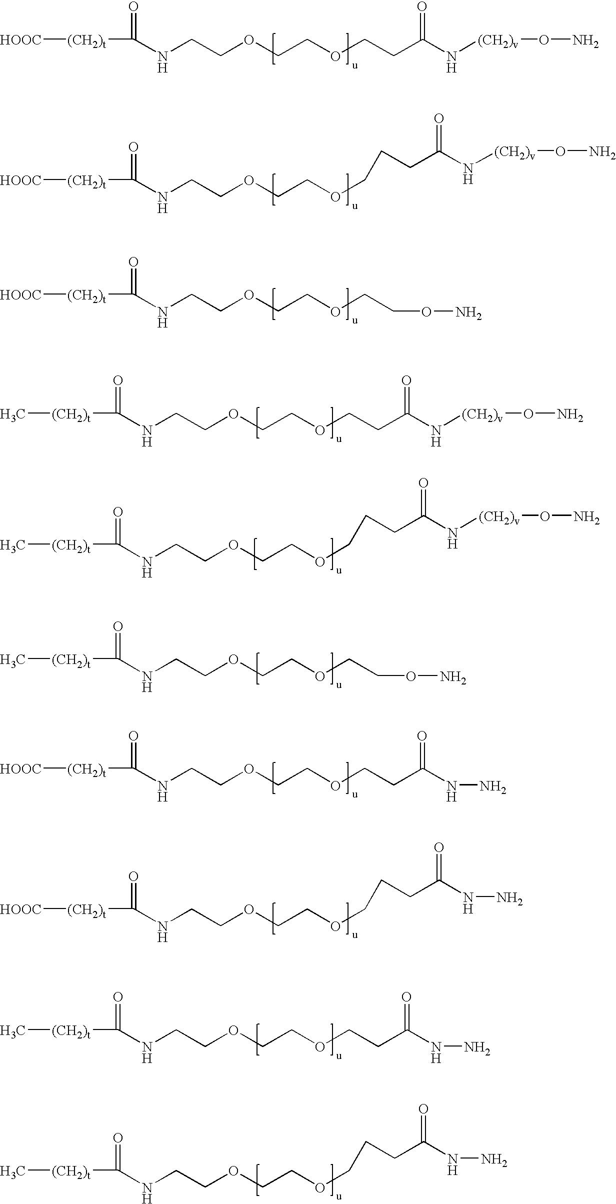

wherein t is an integer between 10 and 24, and v is an integer between 1 and 10, and v is either an integer between 2 and 16 or a range of numbers describing a polydisperse PEG polymer, such that the average molecular weight is between 1-40 kDa, for example PEG₃₄₀₀, PEG₅₀₀₀, PEG₁₀₀₀₀, PEG₂₀₀₀₀, and PEG₄₀₀₀₀.

In one embodiment, reactant X is a nucleophile, which can form a covalent linkage upon dehydration. Non-limiting examples for illustration include hydroxylamines, O-alkylated hydroxylamines, amines, stabilized carbanions, stabilized enolates, hydrazides, alkyl hydrazides, hydrazines, acyl hydrazines, α-mercaptoacylhydrazides etc. Other embodiments includes ring forming (e.g. thiazolidine forming) nucleophiles such as, e.g., thioethanamines, cystein or cystein derivatives.

In some cases (vide infra) the product of the reaction may be further reacted with a reducing agent (a reductant) to form reduced products as indicated below:

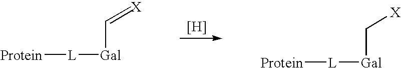

In such cases, and non-limiting, examples of reducing agents (reductants) include sodium cyanoborohydride, pyridine borane, and sodium borohydride, and examples of x includes hydrazides, primary and secondary amines.

In general, O-alkylated hydroxylamine derivatives, when reacted with aldehydes form stable oxime derivatives spontaneously:

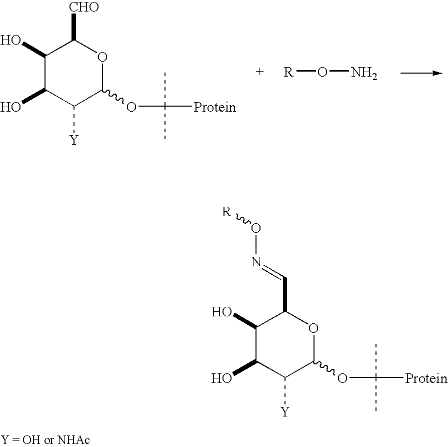

Though more reactive, and in some cases directly destructive to the protein in question, alkyl hydrazines also react efficiently with aldehydes to produce hydrazones. Hydrazones are stable in aqueous solution and may therefore be considered as an alternative to hydroxylamines for derivatization:

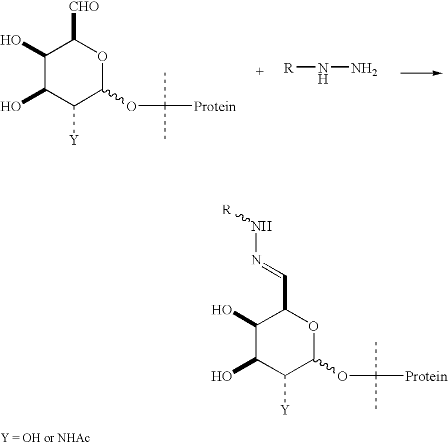

Hydrazides on the other hand, also react spontaneously with aldehydes, but the acyl hydrazone product is less stable in aqueous solution. When using hydrazide derivatived ligands, the resultant hydrazone is therefore frequently reduced to N-alkyl hydrazide using mild reduction reagents such as sodium cyanoborohydride or pyridine borane. See for example Butler T. et al. Chembiochem. 2001, 2(12) 884-894.

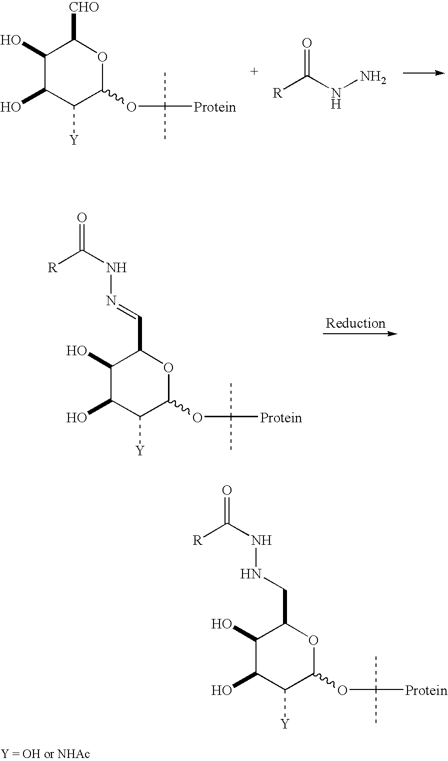

Formation of Schiff-bases between amines and aldehydes offers another type of chemical conjugation methodology. As in the case of hydrazides, a mild reduction of the imine to produce amines is frequently required in order to obtain a stable conjugate.

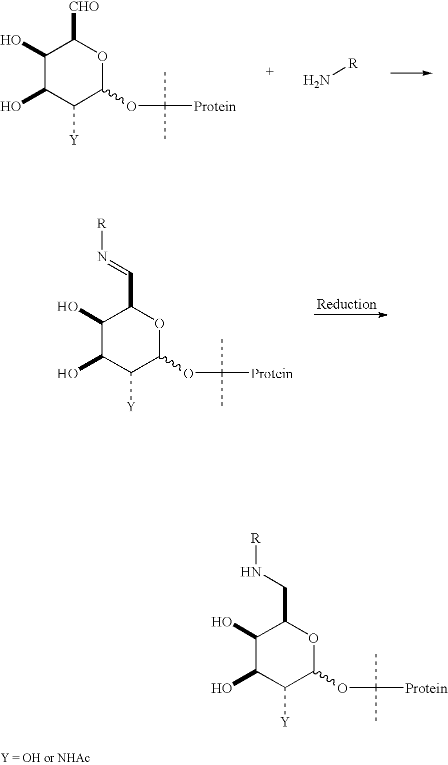

Although mild reduction reagents are known some difficulties in avoiding reduction of sulphide-sulphide (SS) bridges in the protein can be foreseen. In such cases, a chemically conjugation principle that avoid reducing agents is preferred.

C6-oxidized galactose residues also react efficiently with amino thiols such as cystein or cystein derivatives or aminoethane thiol to produce thiazolidines as depicted below:

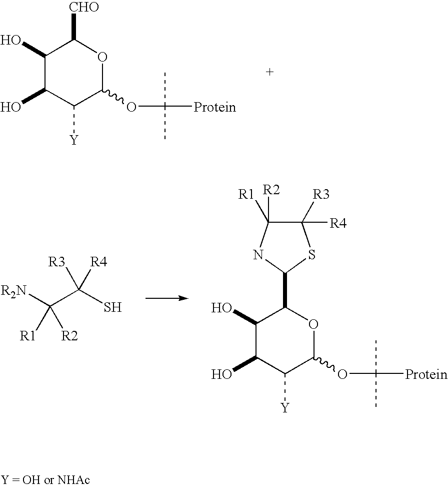

A similar type of modification that also leads to cyclic products involves α-mercaptoacylhydrazides:

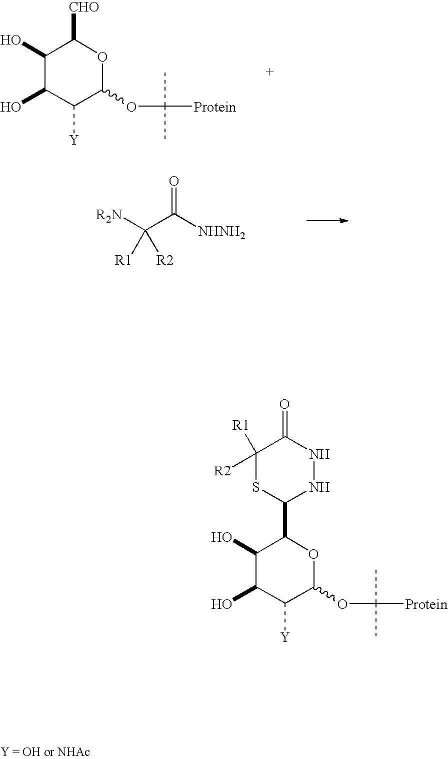

C6-oxidized galactose residues can also react with carbanionic organophosphorus reagents in a Horner-Wadsworth-Emmons reaction. The reaction forms an alkene as depicted below. The strength of the nucleophile can be varied by employing different organophosphorus reagents, like those employed in the Wittig reaction.

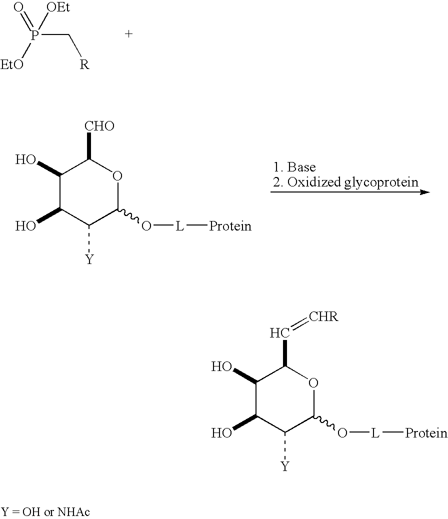

C6-oxidized galactose residues can also react with carbanion nucleophiles. An example of this could be an aldol type reaction as illustrated below. The Z′ and Z″ groups represent electron withdrawing groups, such as COOEt, CN, NO₂ (see March, Advanced Organic Chemistry, 3rd edition, John Wiley & Sons, N.Y. 1985), which increase the acidity of the methylene protons. In the invention, one or both of the Z groups would also be connected to an R group (protractor), which could improve the properties of the glycoprotein.

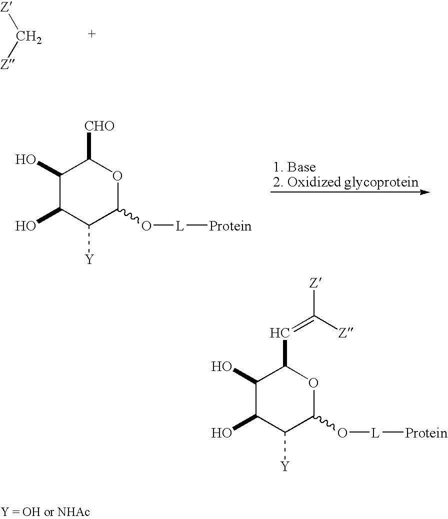

The above listed examples for modifying galactose oxidized in the C6 positions serves as non-limiting examples of the present invention. Other nucleophiles and chemical procedures for modifying aldehyde functions such as those present on C6-oxidized galactose are known to the skilled person (see March, Advanced Organic Chemistry, 3rd edition, John Wiley & Sons, N.Y. 1985).

Linker Molecules

Modification of the oxidized (asialo) glycoprotein, may also proceed in more than one step, before reaching to the final product. Thus, in one embodiment the C6 oxidized galactose residue is initially reacted with a linker molecule possessing specificity for the aldehyde moiety. The linker molecule, itself containing an additional chemical handle (bifunctional), is then reacted further by attaching another molecule (e.g. the protractor group) to give the final product:
Glycoprotein-CHO→Glycoprotein-Linker-X→glycoprotein-Linker-R

Suitable bifunctional linkers are well known to the skilled person, or can easilly be conceived. Examples include, but are not limited to bifunctional linkeres containing hydroxylamine-, amine-, or hydrazied in combination with malimides, succimidyl ester, thiols hydroxylamines, amines, hydrazides or the like.

Although written as stepwise reactions in reaction scheme 1 above, it may in some cases be preferable to add the nucleophile directly into the reaction mixture when performing the oxidation using the galactose oxidase-catalase or the galactose oxidase horseradish peroxidase enzyme couple. Such one-pot conditions can prevent any intermolecular protein protein reactions of the aldehyde functionalities on one protein with the amino groups (e.g. epsilon amines in lysine residues) on the other. Intra and intermolecular Schiff base (imine) formation between proteins can lead to incomplete reaction with the nucleophile, or precipitation of the protein in question. One pot conditions also prevent any possible over-oxidation mediated by galactose oxidase, as the aldehyde functionality instantly can react with the nucleophile present in the reaction media. The concentration ratio of nucleophile to protein may depend on the protein in question and the type of nucleophile (e.g. hydroxylamine, hydrazide, amine, etc.) selected for conjugation. Optimal conditions may be found by experiments, e.g. perform variation in the concentration ratio of nucleophile to protein, perform variation in the overall concentration of protein in solution, etc.

While the galactose or N-acetylgalactosamine residue(s) is/are generally exposed after treatment with sialadase, the invention can be used to covalently bind a protractor group to any terminal galactose moiety. One example could be the addition of terminal galactose residues to a glycan by the use of galactosyl transferases, and such terminal galactose residues could be modified by the technology described by the invention.

Protraction Principle

With the methods described in this invention, a broad selection of known clearance mechanisms for proteins and glycoproteins in particular, can be addressed. Clearance methods for glycoproteins include, without limitation, glomerular filtration and renal clearance, proteolytical degradation by proteases present in plasma, recognition by glycan specific receptors, e.g., in the liver and antibody mediated immunoneutralization.

Glomerular Filtration and Renal Clearance:

Glomerular filtration is an important clearance pathway for proteins. Glomerular filtration is generally considered to be related to size and overall charge characteristics of the protein. The basement membrane of the glomerular capillary wall is an ion filter, composed of negatively charged proteoglycans, which is able to differentiate between neutral blood components i.e. water and urea, and charged blood components such as salts, peptides and proteins.

Charges on the protein surface can interact with the fixed negatively charged heparin sulphate proteoglycan of the glomerular basement membrane. Proteins with surplus of negative surface charges tends to be repulsed by the fixed negatively charged heparin sulphate proteoglycan constituting the glomerular basement membrane and therefore are less likely to pass into the urine. For example, the fractional clearance of negatively charged horseradish peroxidase has been estimated to 0.007, compared to the fractional clearance for neutral horseradish peroxidase which has been estimated to 0.061 (H. G. Rennke, Y. Patel, and M. A. Venkatachalam, Kidney Int. 13 1978 278-288).

Changes of surface charges therefore can have a marked effect on the proteins susceptibility towards glomerular filtration. Thus in one embodiment, a protein is treated with neuraminidase and galactose oxidase according to the invention and then reacted with a molecular ligand containing one or more positively or negatively charged moieties, to produce a protein with altered surface charge properties.

As mentioned, the size of the protein also determines its ability to pass the glomerular capillary wall. Larger proteins (e.g. proteins with molecular mass above 50 KDa) have low tendency for renal clearance compared to smaller proteins. Increasing the size of a given protein thus may increase circulation half life, by minimizing renal clearance.

Therefore, in another embodiment, a protein is treated with neuraminidase and galactose oxidase according to the invention and then reacted with a reactant X, e.g, in the form of a nucleophile, containing a protractor group such as, e.g., PEG or one or more dendrimers to produce a protein with considerable increased size, in order to prevent renal clearance.

Dendrimers:

The following section describes a new class of branched polymers, that are made up of a precise number of monomer building blocks that are oligomerized in any order either on solid support or in solution using suitable monomer protection and activation strategies.

General Structure of Monomers

The monomers of dendrimers are in general linear or branched bi-, tri- or tetrafurcated building blocks of the general structure A-L2-C-(L3-B)ₙ (general formula I) where C serves as attachemt moiety for A-L2 as well as branching moiety for n number of L3-B, in which L2 and L3 both are linker moieties:
A-L₂-C [symbol] L₃-B]ₙ General formula I

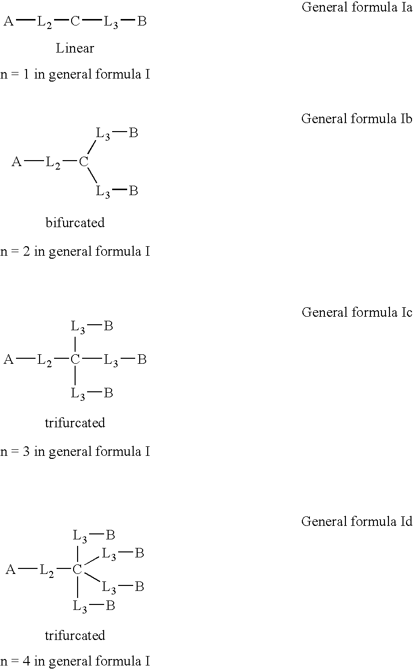

A and B both are functional groups selected in such way, that they together under appropriate condition can form a covalent bond. The nature of the newly formed covalent bond depend upon the selection of A and B, and include but is not limitted to: amide bonds, carbamate bonds, ester bonds, phosphate ester bonds, thiophosphate ester bonds, phosphoramidates, ether, and thioether bonds.

A may be selected from (but is not limitted to): COOH, COOR, OCOOR, OP(NR2)OR, OP(OR)₂, COCl, COBr, OCOCl, OCOBr, CHO, Br, Cl, I, OTs, OMs, alkynes,

Where R is alkyl, aryl or substituted aryl,

Preferably, the moiety A of general formula I, represent an activated moiety that can react with nucleophiles either on the peptide or of type B. Preferably A is selected from the group of:

Functional groups capable of reacting with amino and hydroxy groups such as
a) carbonates such as the p-nitrophenyl, or succinimidyl;
b) carbonyl imidazoles or carbonyl chlorides;
c) carboxylic acids that are activated in situ;
d) carbonyl halides, activated esters such as N-hydroxysuccinimide esters, N-hydroxybenzotriazole esters, esters of 1,2,3-benzotriazin-4(3H)-one
e) phosphoramidites and H-phosphonates or
f) isocyanates or isothiocyanates.

B may be selected from NH₂, OH, N₃, NHR′, OR′, O—NH₂, O—NHR′,

Where R′ is a protection group including, but not limmited to:

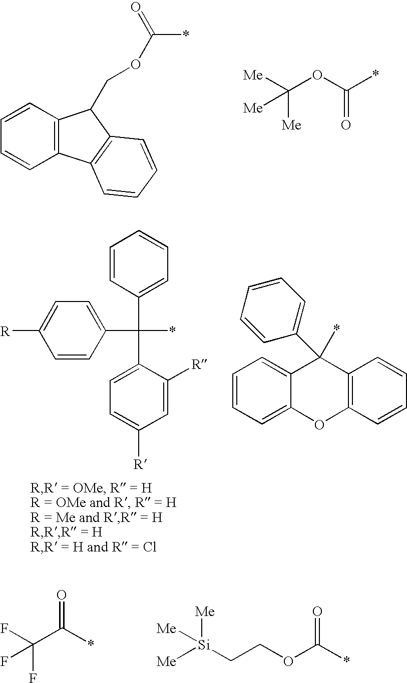

Other examples of appripriate protection groups are known to the skilled person, and suggestions can be found in Green & Wuts “Protection groups in organic synthesis”, 3.ed. Wiley-interscience.

Preferably, the moiety B of general formula I, represent a protected nucleophile moiety that can react with electrophiles preferably of type A. Preferably B is selected from the group of:

a) Fmoc protected amino groups

b) free amino groups

c) azides, that can be reduced to amino groups

d) azides, that may participate together with alkynes to form triazoles

e) O-substituted hydroxylamines

f) hydroxyl groups

g) DMT, MMT or trityl-protected hydroxyl groups

In an other embodiment of the invention, the defintion of A and B may be interchanged to facilitate branched polymer assembly by the convergent approach as described vide infra.

C is either a linear (divalent organic radical) or a branched (multivalent branched organic radical) linker, preferably of hydrophilic nature. It preferably includes a multiply-functionalized alkyl group containing up to 18, and more preferably between 1-10 carbon atoms. Several heteroatoms, such as nitrogen, oxygen or sulfur may be included within the alkyl chain. The alkyl chain may also be branched at a carbon or a nitrogen atom. In one aspect of the invention, C is a single nitrogen atom

Example of C include but is not limitted to divalent organic radicals such as ethylene, arylene, propylene, ethyleneoxy,
or multivalent organic radicals such as propan-1,2,3-triyl, benzen-1,3,4,5-tetrayl, 1,1,1-nitrogentriyl, or a multivalent carbocyclic ring including, but is not limited to the following structures:

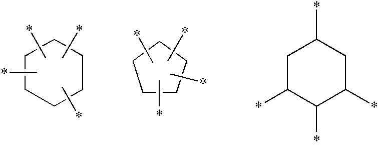

In one embodiment, C is

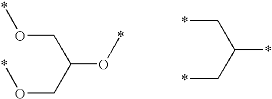

C may be separated from A or B by linker L2 and L3, which preferably are of hydrophilic nature. Examples of such linkers include but is not limmited to
1,2-ethandiyl, 1,3-propandiyl, 1,4-butandiyl, 1,5-pentandiyl, 1,6-hexandiyl, (CH₂CH₂O—)ₙ, where n is an integer between 0 and 10,
—(CR1R2-CR3R4-O)ₙ—, where n is an integer between 0 and 10 and R1, R2, R3 and R4 independently can be H, Me, Et, Pr.
((CH₂)ₘO)ₙ—, where m is . . . 2, 3, 4, 5, 6, and n is an integer between 0 and 10, It is not necessary however, that C is symetrically.
L2, L3 or both can also be a valence bond.

Preferably, L2 and L3 are selected from water soluble organic divalent radicals. Thus, in one embodiment, either L2 or L3 or both are divalent organic radicals containing about 1 to 5 PEG (—CH₂CH₂O—) groups.

In one embodiment, L2 is -oxy- or -oxymethyl-, and L3 is (CH₂CH₂O—)₂:

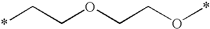

Thus, in one embodiment A is a carboxyl group and B is a protected amino group which after deprotection may be coupled to a new monomer of same type via its carboxy group to form an amide.

In another embodiment, A is a phosphoramidite and B is a hydroxyl group suitable protected, which upon deprotection can be coupled to an other monomer of same type to form a phosphite triester which subsequently are oxidized to form a stable phosphate triester or thio phosphate triester.

In still another embodiment, A is an reactive carbonate such as nitrophenyl carbamate, and B is an amino group, preferably in its protected form.

In yet another embodiment, A is an acyl halide such as COCl or COBr and B is an amino group, preferably in its protected form.

In a specific embodiment, A-L2-C-(L3-B)ₙ is

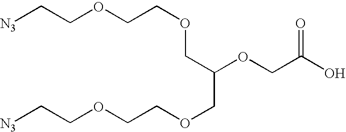

In a specific embodiment, A-L2-C-(L3-B)ₙ is

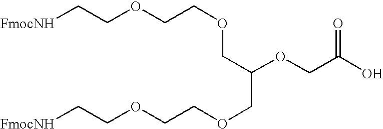

In a specific embodiment, A-L2-C-(L3-B)ₙ is

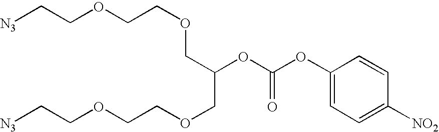

In a specific embodiment, A-L2-C-(L3-B)ₙ is 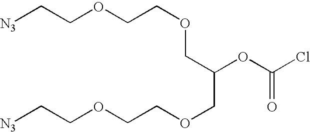
Synthesis of Polymers with Dendritic Structure

Branched polymers can in general be assembled from the monomers described above using one of two fundamentally different oligomerization strategies called the divergent approach and the convergent approach.

Assembly of Branched Polymers by the Divergent Approach:

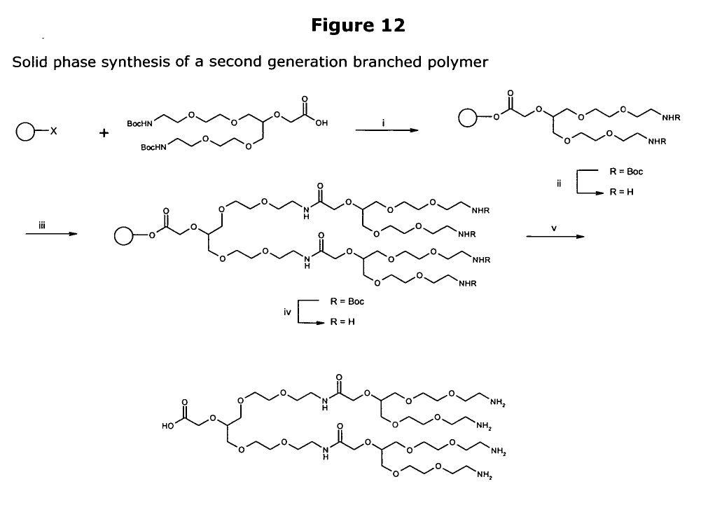

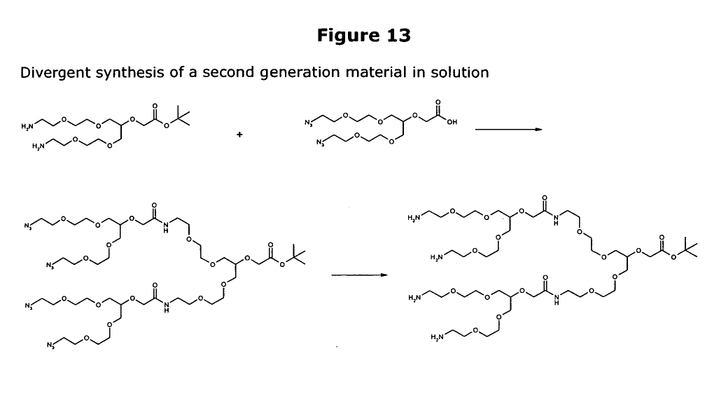

In one embodiment, the branched polymers are assembled by an iterative process of synthesis cycles, where each cycle use suitable activated, reactive bi- tri or multi furcated monomers, them self containing functional end groups—allowing for further elongation (i.e. polymer growth). The functional end groups usually needs to be protected in order to prevent self polymerization and a deprotection step will in such cases be needed in order to generate a functional end group necessary for further elongation. One such cycle of adding a activated (reactive) monomer and subsequent deprotection, in the iterative process completes a generation. The divergent approach is illustrated in FIG. 13 using solution phase chemistry and in FIG. 12 using solid phase chemistry.

Convergent Assembly of Branched Polymers:

However, when higher generations materials are reached in such an itterative process, a high packing density of functional end groups will frequently appear, which prevent further regular growht leading to incomplete generations. In fact, with all systems in which growth requires the reaction of large numbers of surface functional groups, it is difficult to ensure that all will react at each growth step. This poses a significant problem in the synthesis of regular mono dispersed and highly organized branched structures since unreacted functional end groups may lead to failure sequences (truncation) or spurious reactivity at later stages of the stepwise growth sequence.

In one embodiment of the invention, the branched (dendritic) polymer therefore is assembled by the convergent approach described in U.S. Pat. No. 5,041,516. The convergent approach to building macromolecules involves building the final molecule by beginning at its periphery, rather than at its core as in the divergent approach. This avoid problems, such as incomplete formation of covalent bonds, typically associated with the reaction at progressivly larger numbers of sites.

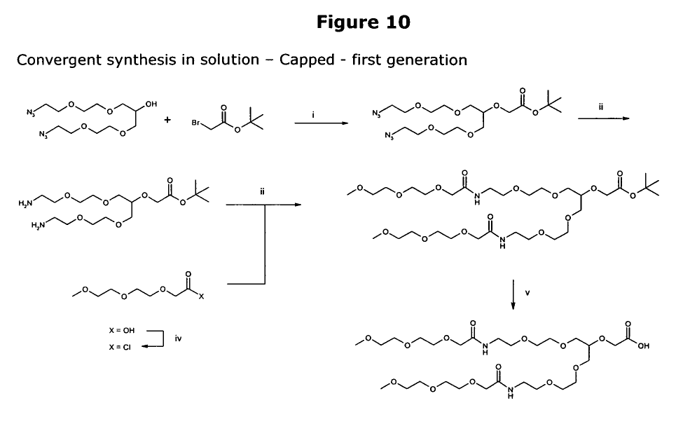

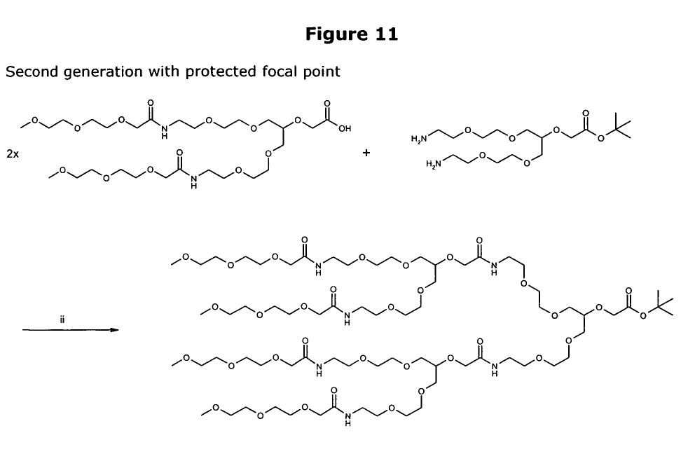

The convergent approach for assembly 2. generation dendritic material is illustrated in FIG. 10 and FIG. 11 using a specific example using one of the monomers of the invention.

Variations in the Individual Layers

It is important to note, that the final branched polymer if desired may consist of different types of monomer building block in each of its layers or generations. By using different monomers in each layer, branched polymers with tailored properties can be made. That way the overall properties of the polymer, and the polymer-peptide conjugate can be controlled.

It could for instance be of interest to control the over all rigidity of the branched polymer. By choosing a bifurcated monomers in the initial layer, followed by one or several layers of linear monomers, a polymer structure with a low number of branches and an overall floppy structure can be created. On the other hand, using a high branched monomer such as a tri- or tetrafurcated monomer repeatingly in each layer, while omitting any linear of low branched monomers, a hyper branched polymer with high density and overall compact structure can be obtained. Rigidity can also be controlled by the design of the particular monomer, for example by using a rigid core structure (C) or by using rigid linker moieties (L2, L3). Fine tuning, or adjustment of the rigity can then be obtained by using the rigid monomer in one or more specific layers intermixed with monomers of more flexible nature. It also could be of interest to fine tune the overall hydrophilic nature of the polymer. This could be realized by choosing monomers with more hydrophobic core structure (C) or more hydrophobic linker moieties (L2 & L3), in one or more of the dendritic layers.

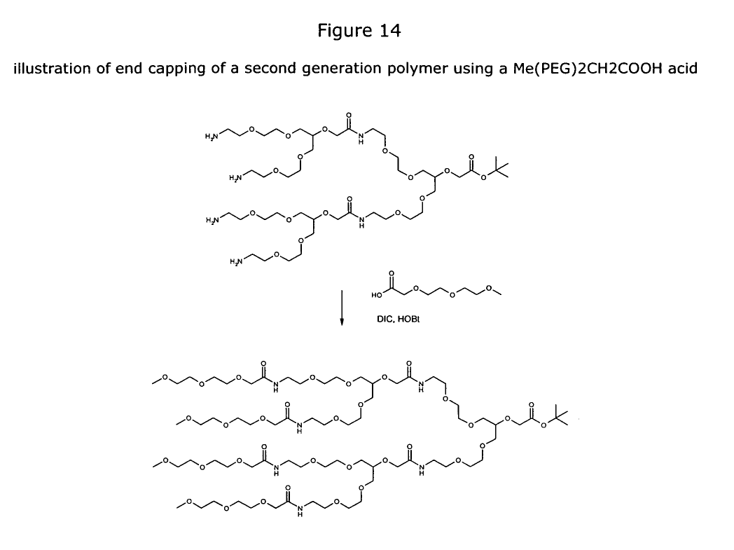

It is also often desirable to use a different monomer in the outer layer of the branched polymer, which in the final peptide conjugate will be exposed to the surounding enviroment. Some of the monomers described in this invention has protected amine functions as terminal end groups (B), which after a deprotection step, and under physiological conditions i.e. neutral physiological buffered pH around 7.4, will be protonated, causing the overall structure to be polycationically charged. Such polycationic structures has been proven to be toxic in animal studies and though they generally are rapidly cleared from the blood circulation system, they should be avoided in any pharmaceutical context. By carefull selection of the monomer used to create the final layer, polycationic structures can be avoided. One example as depicted in FIG. 14, using a Me(Peg)2CH2COOH acid for capping the final layer of a dendritic structure, that otherwise would be terminated in amines.

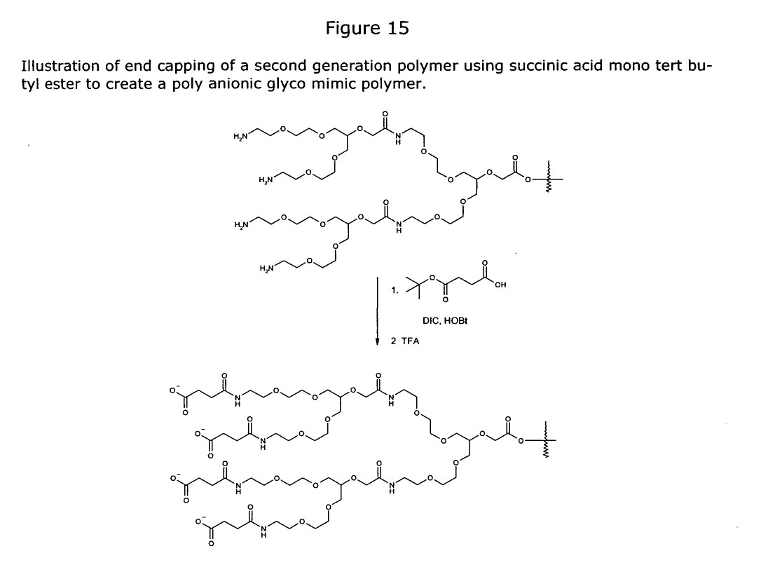

On the other hand, polyanionic biopolymers are well known in e.g. natural occuring glycoproteins, which commonly has multiple anionic charged sialic acids as termination groups on the antenna structure of their N-glucans. Again according to the invention and by proper choice of the monomer used to create the final layer, such glucans can be imitated with respect to their poly anionic nature. One such example is depicted in FIG. 15, where the dendritic layer is capped with succinic acid mono tert butyl estes which upon deprotection with acids renders a polymer surface that are negatively charged under physiological conditions.

Solid Phase Oligomerization

The assembly of monomers into polymers may be conducted either on solid support as described by N. J. Wells, A. Basso and M. Bradley in Biopolymers 47, 381-396 (1998) or in and appropriate organic solvent by classical solution phase chemistry as described by Frechet et al. in U.S. Pat. No. 5,041,516.

Thus in one aspect of the invention, the branched polymer is assembled on a solid support derivatized with a suitable linkage, in an iterative divergent process as described above and illustrated in FIG. 12. For monomers designed with Fmoc or Boc protected amino groups (B), and reactive functional acylating moieties (A), solid phase protocols useful for conventional peptide synthesis can conveniently be adapted. Applicably standard solid phase techniques such as those described in literature (see Fields, ed., Solid phase peptide synthesis, in Meth Enzymol 289) can be conducted either by use of suitable programmable instruments (e.g. ABI 430A) or similar home build machines, or manually using standard filtration techniques for separation and washing of support.

For monomers with e.g. DMT protected alcohol groups (B), and e.g. reactive phosphor amidites (A), solid phase equipment used for standard oligonucleotide synthesis such as Applied Biosystems Expidite 8909, and conditions such as those recently described by M. Dubber and J. M. J. Fréchet in Bioconjugate chem. 2003, 14, 239-246 can conveniently be applied. Solid phase synthesis of such phosphate diesters according to the conventional phosphoramidite methodology requiers that an intermediate phosphite triester is oxidized to a phosphate triester. This type of solid support oxidation, which is convineinetly achieved using iodine in an inert organic solvent, requires that the monomers with or without protection groups resist iodine oxidation. The phosphor amidite methodology also allows for convenient synthesis of thiophosphates by simple replacement of the iodine with elementary sulfur in pyridine or organic thiolation reagents such as 3H-1,2-benzodithiole-3-one-1,1-dioxide (see for example M. Dubber and J. M. J. Fréchet in Bioconjugate chem. 2003, 14, 239-246).

The resin attached branched polymer, when complete, can then be cleaved from the resin under suitable conditions. It is important, that the cleavable linker between the growing polymer and the solid support is selected in such way, that it will stay intact during the oligomerization process of the individual monomers, including any deprotection steps, oxidation or reduction steps used in the individual synthesis cycle, but when desired under appropriate conditions can be cleaved leaving the final branched polymer intact. The skilled person will be able to make suitable choices of linker and support, as well as reaction conditions for the oligomerisation process, the deprotection process and optionally oxidation process, depending of the monomers in question.

Resins derivatized with appropriate functional groups, that allows for attachment of monomer units and later act as cleavable moieties are commercial available (see f.ex the cataloge of Bachem and NovoBiochem).

In an other aspect of the invention, the branched polymer is synthesised on a resin with a suitable linker, which upon cleavage generates a branched polymer product furnished with a functional group that directly can act as an attatchment group in a subsequent solution phase conjugation process to a peptide as described below, or alternatively, by appropriate chemical means can be converted into such an attachment group.

Solution Phase Oligomerization

In some cases, it can be advantageous to synthesise dendritic branched polymers of a certain size and compositions using classical solution phase techniques.

Therefore, in another aspect of the invention, the branched polymer is assembled in an appropriate solvent, by sequential addition of suitable activated monomers to the growing polymer. After each addition, a deprotection step may be needed before construction of the next layer or generation can be initiated. It may be desirable to use excess of monomer in order to reach complete reactions. In one aspect of the invention, the removal of excess monomer takes advantages of the fact that hydrophilic polymers have low solubility in diethyl ether or similar types of solvents. The growing polymer can thus be precipitated leaving the excess of monomers, coupling reagents, biproducts etc. in solution. Phase separation can then be performed by simple decantation, of more preferably by centrifugation followed by decantation. Polymers can also be separated from biproducts by conventional chromatographic techniques on e.g. silica gel, or by the use of HPLC or MPLC systems under either normal or reverse phase conditions as described in P. R. Ashton et al. J. Org. Chem. 1998, 63, 3429-3437. Alternatively, the considerbly larger polymer can be separated from low molecular components, such as excess monomers and biproducts using size exclusion chromatography optionally in combination with dialysis as described in E. R. Gillies and J. M. J. Fréhet in J. Am. Chem. Soc. 2002, 124, 14137-14146.

In contrast to solid phase techniques, solution phase also makes it possible to use the convergent approach for assembly of branched polymers as described above and further reviewed in S. M. Grayson and J. M. J. Fréchet, Chem. Rev. 2001, 101, 3819-3867. In this approach it is desirable to initiate the synthesis with monomers, where the protected functional end groups (B) initially is converted into moieties that eventually will be present on the outer surface of the final branched polymer. Therefore the functional moiety (A) of general formula I in most cases will need suitable protection, that allows for stepwise chemical manipulation of the end groups (B). Protection groups for the functional moiety (A) depend on the actually functional group. For example, if A in general formula I is a carboxyl group, a tert-butyl ester derivate that can be removed by TFA would be an appropriate choice. Suitable protection groups are known to the skilled person, and other examples can be found in Green & Wuts “Protection groups in organic synthesis”, 3.ed. Wiley-interscience. The convergent assembly of branched polymers is illustrated in FIG. 10 and FIG. 11. In step (i) of FIG. 10, a tertbutyl ester functionallity (A) is prepared by reaction of a suitable precurser with t-butyl α-bromoacetate. In step (ii) the terminal end groups (B) is manipulated in such way that they allows for the acylation of step (iii), with a carboxylic acid that is converted into a acyl halid in step (iv). In step (v) the t-butyl ester functionality (A) is removed creating a end (B) capped monomer. This end capped monomer serves as starting material for preparing the second generation product in FIG. 11, where 2 equivalents is used in an acylation reaction with the product of step (ii) in FIG. 10. The product of this reaction is a new t-butyl ester, which after deprotection can re-enter in the initial step of FIG. 11 in a itterative manner creating higher generation materials.

Conjugation to Peptides and Proteins

Attachment Moieties on the Branched Polymer

To effect covalent attachment of the polymer molecule(s) to the peptide in solution, the polymer must be provided with a reactive handle, i.e. furnished with a reactive functional group examples of which includes by illustration and not limitation, primary amino groups, hydrazides, hydrazides, β- and γ aminothiols or a hydroxylamine.

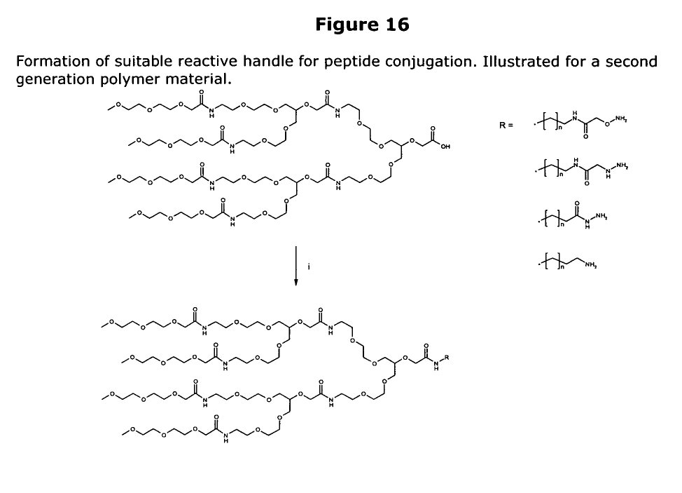

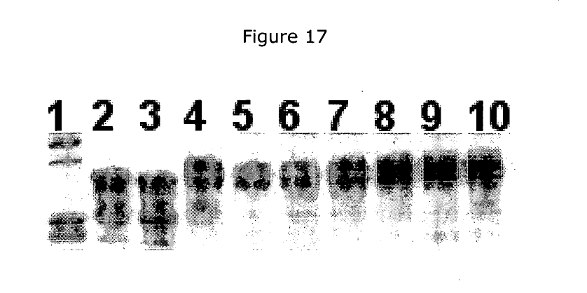

Suitable attachment moieties on the branched polymer, such as those mentioned above, may be created after the polymer has been assembled using either conventional solution phase chemistry or solid phase chemistry. Non-limitted examples on ways to create nucleophilic attachment moieties on a branched polymer containing a carboxylic acid group are listed in FIG. 16

One or more of the activated branched polymers can be attached to a biologically active polypeptides by standard chemical reactions according to the invention. The conjugate is represented by the general formula II:
(branched polymer)_{z}-L⁴-(polypeptide) (formula II)
wherein (branched polymer) is a water-soluble substantially nonantigenic polymer consisting of monomers according to general formula I, L⁴ is an linking moiety essentially defined as for L² and L³ of general formula I, (z) is an integer ≧1 representing the number of branched polymers conjugated to the biologically active polypeptide. The upper limit for (z) will be determined by the number of available attachment sites on the polypeptide, and the degree of polymer attachment sought by the artisan.

The degree of conjugation can, as previously mentioned, be modified by varying the reaction stoiehiometry using well-known techniques. More than one branched polymer conjugated to the polypeptide can be obtained by reacting a stoiehiometric excess of the activated polymer with the polypeptide.

The biologically active polypeptide can be reacted with the activated branched polymers in an aqueous reaction medium which can be buffered, depending upon the pH requirements of the polypeptide. The optimum pH for the reaction is generally between about 6.5 and about 8 and preferably about 7.4 for most polypeptides.

The optimum reaction conditions for the polypeptides stability, reaction efficiency, etc. is within level of ordinary skill in the art. The preferred temperature range is between 4° C. and 37° C. The temperature of the reaction medium cannot exceed the temperature at which the polypeptide may denature or decompose. It is preferred that the polypeptide be reacted with an excess of the activated branched polymer. Following the reaction, the conjugate is recovered and purified such as by diafiltration, column chromatography including size exclussion chromatotrapy, ion-exchange chromatograph, affinity chromatography, electrophoreses, or combinations thereof, or the like.

Specific Examples of Suitable Protected Monomers Included in the Invention:

General Formula Ia—Linear Monomers (A-L2-C-L3-B): 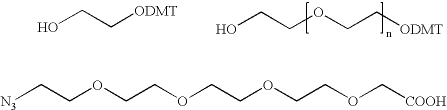
General Formula Ib—Bifurcated Monomers (A-L2-C-(L3-B)₂): 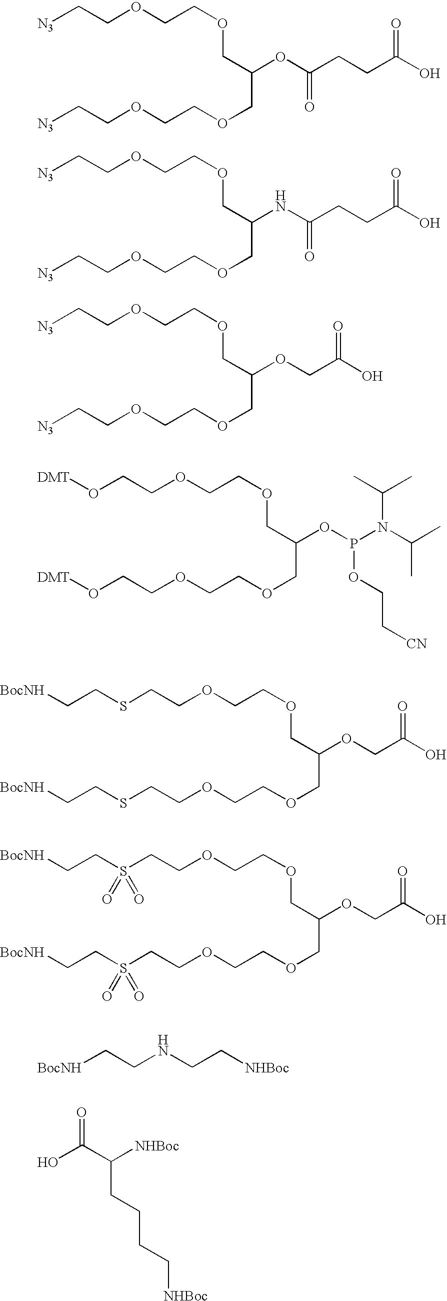
General Formula Ic—Trifurcated Monomers A-L2-C-(L3-B)₃): 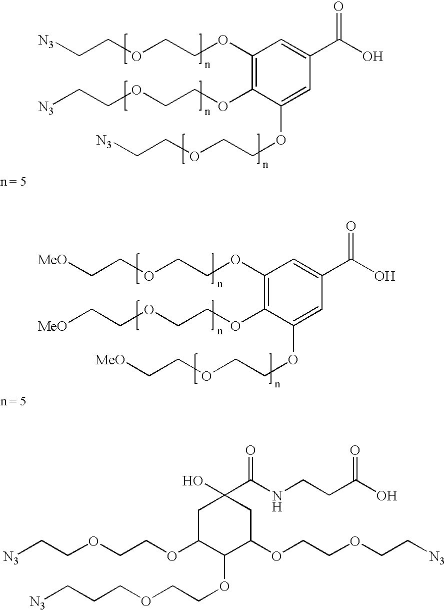
Proteolytical Shielding:

A major problem when using proteins and peptides as therapeutics, is their susceptibility to proteolytical degradation by proteases present in plasma. Degradation plays an obvious role in the elimination of damaged or abnormal polypeptides, but also affects half-lives of normal proteins and peptides, and individual turnover rates can be strongly dependent on the peptide sequence, the structure and in the case of proteins, the surface properties.

Shielding effects against proteolysis not only rely on structural or sequential factors but also global biophysical factors, such as overall hydrophobicity or overall charge characteristics.

Consequently even small changes in overall charge properties of the protein can have effect on protease mediated proteolysis in serum. Such changes in glycan moieties of glycoproteins, by e.g. incorporating moieties that are charged under physiological conditions are included in the invention.

Non-limiting examples of moieties that are negatively charged under physiological conditions includes carboxylic acids, sulfonic acids, phosphonic acids, phosphates, phosphoramidates ect. Non-limiting examples of moieties that are positively charges under physiological conditions includes primary-, secondary-, tertiary- and quaternary amino groups, guanidines, and heterocycles such as pyridine, imidazole, quinoline, etc.

Thus in one aspect of the invention, a glycoprotein treated with sialidase (optionally) and galactose oxidase according to the invention, is reacted with a nucleophile containing a moiety which is either positively or negatively charged under physiological conditions, in order to create a modified glycoprotein with enhanced stability towards serum proteases.

Other alternatives that can increase stability towards proteolytical degradation includes, the attachment of hydrophobic side chains such as long chain alkanes and polyaromates, or attaching bulky hydrophilic polymers such as, e.g., polyethyleneglycol (PEG).

Attaching Albumin Binders

Alternatively, ligands that bind to serum proteins such as albumins may be conjugated to the glycan part of a glycoprotein therapeutic using the procedures described in this invention. The glycoprotein when modified in such way will thereby be able to form a specific non covalent complex to e.g. albumin, which due to the overall size can escape glomerular filtration. As such non covalent complexes may reduce the biological activity of the glycoprotein counterpart; it may be desirable to adjust the affinity of the ligand to the particular serum protein chosen, in order to retain biological activity.

Thus in another aspect of the invention, a glycoprotein treated with galactose oxidase according to the invention, is reacted with a reactant X, e.g., in the form of a nucleophile, containing a long chain fatty acid residue (e.g. C12, C14, C16 or C18) which can bind to human serum albumin, or diacids or lithocholic acids.

In another aspect of the invention, a glycoprotein treated with galactose oxidase is treated, according to the invention, with a reactant X, e.g., in the form of a nucleophile, containing a portion of a protein having a long circulating half-life, such as an immunoglobulin. In a particular embodiment, the glycoprotein is treated with a reactant X, e.g., in the form of a nucleophile, containing a Fc domain of IgG

Shielding of Epitopes—Inhibition of Sugar Specific Recognition Processes:

Mammalian glycoproteins often have N-acetyl-neuraminic acid (sialic acid) as the external (terminal) residue of the oligosaccharide chains which may be N-linked or O-linked (See, e.g., Osawa and Tsuji (1987) Ann. Rev. Biochem. 56:21). Where the nature of the oligosaccharide is the primary determinant for clearance from circulation, generally glycoproteins with terminal sialic acid residues removed (asialoglycoproteins) are cleared more quickly than their intact counterparts. Circulating glycoproteins are exposed to sialidase(s) (or neuraminidase) which can remove terminal sialic acid residues. Typically the removal of the sialic acid exposes galactose residues, and these residues are recognized and bound by galactose-specific receptors in hepatocytes (reviewed in Ashwell and Harford (1982) Ann. Rev. Biochem. 51:531). The liver also contains other sugar-specific receptors which mediate removal of glycoproteins from circulation. Specificities of such receptors also include N-acetylglucosamine, mannose, fucose and phosphomannose. Glycoproteins cleared by the galactose receptors of hepatocytes undergo substantial degradation and then enter the bile; glycoproteins cleared by the mannose receptor of Kupffer cells enter the reticuloendothelial system (reviewed in Ashwell and Harford 10 (1982) Ann. Rev. Biochem. 51:53). Studies with asialo-ceruloplasmin and derivatives showed that asialo-ceruloplasmin in which galactose residues were oxidized by treatment with galactose oxidase and horseradish peroxidase and asialoagalacto-ceruloplasmin exhibited extended circulating half-lives as compared with asialo-ceruloplasmin (Morell et al. (1968) J. Biol. Chem. 243:155). Efficient removal by the galactose receptor appears to require at least two exposed galactose residues. From the foregoing cited examples of glycoproteins for which sialylation is the key determinant of clearance from circulation and those for which sialylation has no bearing on clearance or for which oligosaccharides play a relatively insignificant role in clearance, one may conclude that the fate of a particular glycoprotein in circulation and its apparent mechanism for clearance most be determined empirically.

Modifications that result in increased half-life include, but are not limited to, exposure of galactose residues followed by oxidation or derivatization of the galactose such that binding of the modified glycoprotein to galactose receptor is inhibited or blocked. Thus in one aspect of the invention, a glycoprotein treated with galactose oxidase according to the invention, is reacted with a nucleophile containing a steric moiety that prevents glycan specific receptor-recognition.

One embodiment is one in which the terminal sialic acid residues of the oligosaccharide side chains of the soluble protein or soluble protein derivative have been removed with neuraminidase treatment, and then the exposed galactose residues are oxidized by galactose oxidase (optionally also with horseradish peroxidase treatment). The oxidation of the exposed galactose residues has the effect of preventing rapid clearance of the modified glycoprotein from circulation by specific galaetose receptors in the liver. It is understood that other structural modifications of terminal galactose residues, including but not limited to addition of a functional group or small molecule or mild oxidation treatment, which have the effect of blocking, inhibiting or preventing recognition of terminal galactose residues without destroying the biological activity are functionally equivalent.

Modified glycoproteins made in accordance with the present invention include those with structural alterations (modifications) of the oligosaccharide portions of the glycoprotein which result in prolonged circulating half-life by blocking or inhibiting clearance via sugar-specific receptors of the liver, by reducing renal clearance, or by minimizing proteolytically degradation. Modifications which substantially decrease the biological activity of the glycoprotein are to be avoided.

Preferably the glycoproteins are synthesized in a recombinant mammalian host. The chemical and enzymatic treatments to produce the structural modifications of the oligosaccharide should not substantially alter the binding reaction of the glycoprotein to its biological target protein. It is preferred that the circulating half-life of modified glycoprotein of the present invention be at least about. 4-48 hours, such as at least 24 hours in humans (or at least about 2-12 hours, such as at least about 6 hours as measured in a rat animal model). It is most preferable that the structural modification of the oligosaccharide so as to prolong circulating half-life is the most conservative structural change which will achieve the end. A variety of chemical derivatization procedures, or chemical and/or enzymatic procedures, as understood in the art, may be employed to produce the modified glycoprotein of the present invention.

For any modified glycoprotein made in accordance with the present invention it is most desirable that an immunological response will not be elicited in a human patient exposed to the modified glycoprotein. It is also required that the biological activity is not significantly decreased to detrimentally affect the therapeutic function of the glycoprotein by the structural modification employed to confer prolonged circulation. It is also most desirable, that any structural modification of a glycoprotein does not result in toxicity in a patient to which that modified glycoprotein is administered. Clearly for use in therapeutics, the modified glycoprotein should have minimal toxic, irritant or other side effects upon administration to humans. Strategies for prolonging the circulation of a particular glycoprotein must therefore be evaluated on a case-by-case basis.

Chemical modification or derivatization of the C6 position of galactose is preferred to maximize half-life and minimize clearance without significantly affecting biological function and without eliciting negative physiological reactions; minimal and mild treatment is preferred. Functional groups or other structure-modifying molecules that may be added to the oligosaccharide portion of a glycoprotein in accordance with the present invention include any chemical group from the size of a single methyl group to larger polymeric groups such as, e.g., polyethylene glycol. Modifications which substantially decrease the biological activity of the glycoprotein are to be avoided.

In general, the mechanism for clearance most be evaluated and the strategies for slowing or avoiding clearance must take into account maintenance of desired biological activity or function, potential toxicity, potential immunogenicity and cost.

A uniform modified glycoprotein may be incorporated in a therapeutic composition or a mixture of modified glycoprotein may be formulated in such a composition, so long as the desired therapeutic action is achieved by those molecules and so Iong as clearance by sugar-specific receptors mediating clearance, by proteolysis or by renal clearance, is inhibited or prevented by the modification or modifications made to said glycoprotein. Thus, the methodology is generally applicable to therapeutic glycoproteins which are cleared from circulation by renal clearance, sugar specific receptors, or by proteases present in serum. It will be readily apparent that those of ordinary skill in the art that assays, reagents, procedures and technique other than those specifically described herein, can be employed to obtain the same or equivalent results and achieve the goals described herein. For example, chemical means of oxidation by removal of sialic acid can be readily substituted for enzymatic means specifically described. All such alternatives are encompassed by the spirit and scope of this invention.

In one embodiment of this invention, a modified soluble glycoprotein derivative with increased plasma half-life as compared with the unmodified derivative is produced. Increased circulating half-life of glycoproteins is achieved in general by means which block or inhibit removal of glycoprotein by galactose, mannose or other sugar-specific receptors, or by means that inhibits renal clearance, proteolytical degradation or immunological neutralization.

Thus in another embodiment, a protein containing an biantennary N-glucan is reacted with sialidase, galactose oxidase and a hydrogen peroxide scavenger (e.g., catalase) according to the invention and the resultant protein product treated with O-pegylated hydroxylamine to produce a glycol-glycoconjugate with reduced renal clearance.

In another embodiment, a galactose oxidized glycoprotein prepared according to the invention is reacted with O-carboxymethyl hydroxylamine to produce a glycoprotein with isoelectric properties similar to wild type glycoprotein, but without any sialidase labile neuraminic acids.

In still another embodiment, a galactose oxidized glycoprotein prepared according to the invention is reacted with O-diethylaminoethyl hydroxylamine to produce a glycoprotein with altered binding properties to the hepatic lectine receptors.

In one embodiment, a glycoprotein is optionally reacted with a sialidase followed by galactose oxidase and a nucleophile to produce a glycoconjugate with reduced renal clearance compared to the unmodified glycoprotein. In one embodiment thereof, the produced glycoconjugate has a reduction in renal clearance of at least 50% compared to the unmodified glycoprotein. In another embodiment thereof, the produced glycoconjugate has a reduction in renal clearance of at least 100% compared to the unmodified glycoprotein.

In another embodiment, a glycoprotein is optionally reacted with a sialidase followed by galactose oxidase and a nucleophile to produce a glycoconjugate with reduced binding to the asialoglycoprotein receptor compared to the unmodified glycoprotein.

In another embodiment, a glycoprotein is optionally reacted with a sialidase followed by galactose oxidase and a nucleophile to produce a glycoconjugate with reduced binding to the mannose receptor compared to the unmodified glycoprotein.

In another embodiment, a glycoprotein is optionally reacted with a sialidase followed by galactose oxidase and a nucleophile to produce a glycoconjugate with reduced clearence by the liver compared to the unmodified glycoprotein. In one embodiment thereof, the produced glycoconjugate has a reduction in liver clearance of at least 50% compared to the unmodified glycoprotein. In another embodiment thereof, the produced glycoconjugate has a reduction in liver clearance of at least 100% compared to the unmodified glycoprotein.

In another embodiment, a glycoprotein is optionally reacted with a sialidase followed by galactose oxidase and a nucleophile to produce a glycoconjugate with increased serum stability compared to the unmodified glycoprotein.

In another embodiment, a glycoprotein is optionally reacted with a sialidase followed by galactose oxidase and a nucleophile to produce a glycoconjugate with increased circulation half-life in an animal model compared to the unmodified glycoprotein. In one embodiment thereof, the produced glycoconjugate has 100% increased circulation half-life in an animal model compared to the unmodified glycoprotein.

In one series of embodiments the galactose oxidized glycoprotein prepared according to the invention is reacted with a nucleophile which is connected to a fatty acid (e.g. C₅-C₂₄). The fatty acid can be connected to the nucleophile through a linker moiety. The linker moiety can either be a simple structure designed to connect the fatty acid to the nucleophile, or it may contain functional groups (e.g. carboxylic acids, amines, alcohol, etc.) which enhance the in vivo properties of the embodiment.

In another series of embodiments the galactose oxidized glycoprotein prepared according to the invention is reacted with a nucleophile which is connected to an aliphatic diacid (e.g. C₅-C₂₄). The aliphatic diacid can be connected to the nucleophile through a linker moiety. The linker moiety can either be a simple structure designed to connect the aliphatic diacid to the nucleophile, or it may contain functional groups (e.g. carboxylic acids, amines, alcohol, etc.) which enhance the in vivo properties of the embodiment.

In another series of embodiments the galactose oxidized glycoprotein prepared according to the invention is reacted with a reactant X, e.g., in the form of a nucleophile, which is connected to a structure that binds to serum proteins, like albumin. The structure that binds to serum proteins can be connected to the nucleophile through a linker moiety. The linker moiety can either be a simple structure designed to connect the aliphatic diacid to the nucleophile, or it may contain functional groups (e.g. carboxylic acids, amines, alcohol, etc.) which enhance the in vivo properties of the embodiment.

In another series of embodiments the galactose oxidized glycoprotein prepared according to the invention is reacted with a reactant X, e.g., in the form of a nucleophile, which is connected to a structure (e.g. sialic acid derivatives) which inhibits the glycans from binding to receptors (e.g. asialoglycoprotein receptor and mannose receptor) that may remove the glycoprotein from circulation.

In another series of embodiments, the glycoprotein is first treated with a series of enzymes e.g. neuramidases, galactosidases, mannosidases, endo H and endo F3 sequentially, or directly by endo H or endo F3 to remove part of the glycan structure (see for example K. Witte et al. J. Am. Chem. Soc., 119, 2114 (1997)). A galactose moiety can then be added to the new terminus by employing a galactosyltransferase and the appropriate galactose substrate, or a series of transferases together with various carbohydrate substrates may be employed before employing the galactosyltransferase. The new glycan structure can then be oxidised and reacted with a nucleophile as described by the invention, thus yielding a glycoprotein with both an alternative glycan structure and a galactose modification which improves its therapeutic properties.

Glycoproteins Suitable for Conjugation in Accordance with the Present Invention

The peptides conjugated with the protractor group are described as “biologically active”. The term, however, is not limited to physiological or pharmacological activities. For example, some inventive polymer conjugates containing proteins such as immunoglobulin, enzymes with proteolytical activities and the like are also useful as laboratory diagnostics, i.e., for in vivo studies etc. A key feature of all of the conjugates is that at least same portion of the activity associated with the unmodified bio-active peptide is maintained.

The conjugates thus are biologically active and have numerous therapeutic applications. Humans in need of treatment which includes a biologically active peptide can be treated by administering an effective amount of a branched polymer conjugate containing the desired bioactive peptide. For example, humans in need of enzyme replacement therapy or blood factors can be given branched polymer conjugates containing the desired peptide.

Biologically active peptides of interest of the present invention include, but are not limited to proteins, peptides, peptides and enzymes. Enzymes of interest include carbohydrate-specific enzymes, proteolytic enzymes. oxidoreductases, transferases, hydrolases, lyases. isomerases and ligasese. without being limited to particular enzymes, examples of enzymes of interest include asparaginase, arginase, arginine deaminase, adenosine deaminase, superoxide dismutase. endotoxinases. cataiases. chymotrypsin, lipases, uricases, adenosine diphosphatase. tyrasinases, and bilirubin oxidase. Carbohydrate-specific enzymes of interest include glucose oxidases, glycosidases, glucocerebrosidases. glucouronidases. etc.

Peptides and proteins, that do not contain glycan moieties can be glycosylated either enzymatically as described in L1 Shao et all. Glycobiology 12(11) 762-770 (2002) using glycosyltransferases, or chemically synthesised, for example by using standard peptide chemistry and glycosylated amino acid components such as N-galactosylated asparagine.

Alternatively glycosylation sites may be engineered into proteins or peptides which in vivo normally are produced in their non-glycosylated form. For example insertion of the consensus sequence Cys-XXX-Ser-XXX-Pro-Cys in an EGF repeat allows for selective O-glycosylation of serine using UDP-Glucose and glucosyltransferase L1 Shao et all. Glycobiology 12(11) 762-770 (2002), whereas insertion of the consensus sequence Asn-XXX-Ser/Thr allows for N-glycosylation R. A. Dwek, Chem. Rev. 1996, 96, 683-720. Peptide sequences containing threonine or serine also undergoes glycosylation in the presence of UDP-GalNAc:polypeptide N-acetylgalactosaminyltransferase and UDP-GalNAc in a sequence dependent manner (see for example B. C. O'Connell, F. K. Hagen and L. A. Tabak in J. Biol. Chem. 267(35), 25010-25018 (1992)). Alternatively site directed mutagenesis introducing cystein mutations can be used for introsuction of galactose or galactose containing sugar structures via mixed disulphide fromation as described by D. P. Gamblin et al. in Angew. Chem. Int. Ed., 43, 828 (2004). Galactose or N-acetylgalactosamine containing peptide and proteins can also be made by conjugation to proteins or peptides containing non-biogenical handles such as methods described by P. G. Schultz in J. Am. Chem. Soc, 125, 1702 (2003), or unspecifically by direct glycosylation of peptides using glycosyl donor substrates such as trichloroacetamidyl galactosides ect. Addition of glycosidase inhibitores to fermentation cultures, thereby producing glycoproteins with truncated glycan structures as described in U.S. Pat. No. 4,925,796A/U.S. Pat. No. 5,272,066A1 is also a possibility for obtaining galactose or N-acetylgalactosamine containing proteins, as well as enzymatic modification of glutamine residues using TGase (see for example M. Sato et al. Angew. Chem. Int. Ed. 43, 1516-1520, (2004).

Production og N-glycosylated proteins are not limited to the use of mammalian host cells such as CHO or BHK cells, but also can be performed using bacterial cells as described by M. Wacker et al. in Science, 298, 1790-1793 (2002).

Proteins and peptides of interest include, but are not limited to, hemoglobin, serum proteins such as blood factors including Factors VII, FX, FII, FV, protein C, protein S, tPA, PAI-1, tissue factor, FXI, FXII, and FXIII, as well as sequence FVIII, FIX variants thereof; immunoglobulins, cytokines such as interleukins, alpha-, beta-, and gamma-interferons, colony stimulating factors including granulocyte colony stimulating factors, platelet derived growth factors and phospholipase-activating protein (PUP). Other proteins and peptides of general biological and therapeutic interest include insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and the like. Immunoglobulins of interest include IgG, IgE. IgM. IgA, IgD and fragments thereof.

In one embodiment of the invention, the glycoprotein is selected from the group consisting of: aprotinin, tissue factor pathway inhibitor or other protease inhibitors, insulin or insulin precursors, human or bovine growth hormone, interleukin, glucagon, oxyntomodulin, GLP-1, GLP-2, IGF-I, IGF-II, tissue plasminogen activator, transforming growth factor γ or β, platelet-derived growth factor, GRF (growth hormone releasing factor), human growth factor, immunoglobulines, EPO, TPA, protein C, blood coagulation factors such as FVII, FVIII, FIX, FX, FII, FV, protein C, protein S, PAI-1, tissue factor, FXI, FXII, and FXIII, exendin-3, exentidin-4, and enzymes or functional analogues thereof. In the present context, the term “functional analogue” is meant to indicate a protein with a similar function as the native protein. The protein may be structurally similar to the native protein and may be derived from the native protein by addition of one or more amino acids to either or both the C and N-terminal end of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Furthermore the protein may be acylated in one or more positions, see, e.g., WO 98/08871, which discloses acylation of GLP-1 and analogues thereof, and WO 98/08872, which discloses acylation of GLP-2 and analogues thereof. An example of an acylated GLP-1 derivative is Lys26(N^{epsilon}-tetradecanoyl)-GLP-1 (7-37) which is GLP-1 (7-37) wherein the epsilon-amino group of the Lys residue in position 26 has been tetradecanoylated.

In one embodiment of the invention, the glycoprotein is selected from the group consisting of: FVII, FVIII, FIX, FX, FII, FV, protein C, protein S, tPA, PAI-1, tissue factor, FXI, FXII, FXIII, as well as sequence variants thereof; immunoglobulins, cytokines such as interleukins, alpha-, beta-, and gamma-interferons, colony stimulating factors including granulocyte colony stimulating factors, platelet derived growth factors and phospholipase-activating protein (PUP). Other proteins and peptides of general biological and therapeutic interest include insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFa's or TGFps and epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, tissue plasminogen activator, and the like. Immunoglobulins of interest include IgG, IgE. IgM. IgA, IgD and fragments thereof.

In one embodiment of the invention, the glycoprotein is FVII. In one embodiment of the invention, the glycoprotein is FVIII. In one embodiment of the invention, the glycoprotein is FIX. In one embodiment of the invention, the glycoprotein is FXIII.

The proteins or portions thereof can be prepared or isolated by using techniques known to those of ordinary skill in the art such as tissue culture, extraction from animal sources, or by recombinant DNA methodologies. Transgenic sources of the proteins, peptides, amino acid sequences and the like are also contemplated. Such materials are obtained form transgenic animals. i.e., mice, pigs, cows, etc., wherein the proteins expressed in milk, blood or tissues. Transgenic insects and baculovirus expression systems are also contemplated as sources. Moreover, mutant versions, of proteins, such as mutant TNF's and/or mutant interferons are also within the scope of the invention. Other proteins of interest are allergen proteins such as ragweed, Antigen E, honeybee venom, mite allergen, and the like.

The foregoing is illustrative of the biologically active peptides which are suitable for conjugation with a protractor group in accordance with the invention. It is to be understood that those biologically active materials not specifically mentioned but having suitable peptides are also intended and are within the scope of the present invention.

In one embodiment of the invention the protein is not CD4 protein; in another embodiment the protein is not soluble CD4 protein.

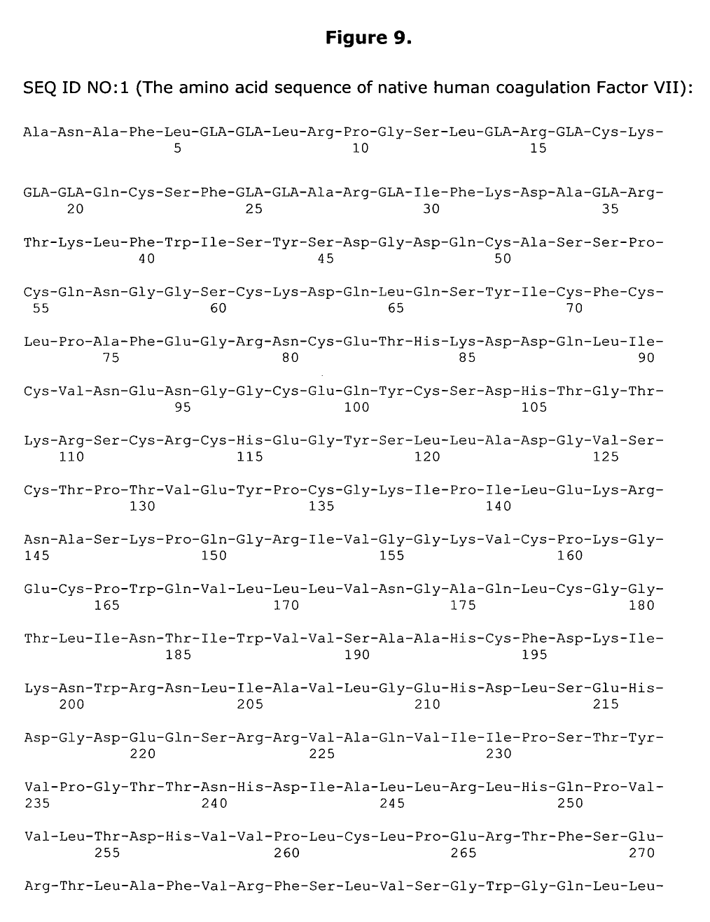

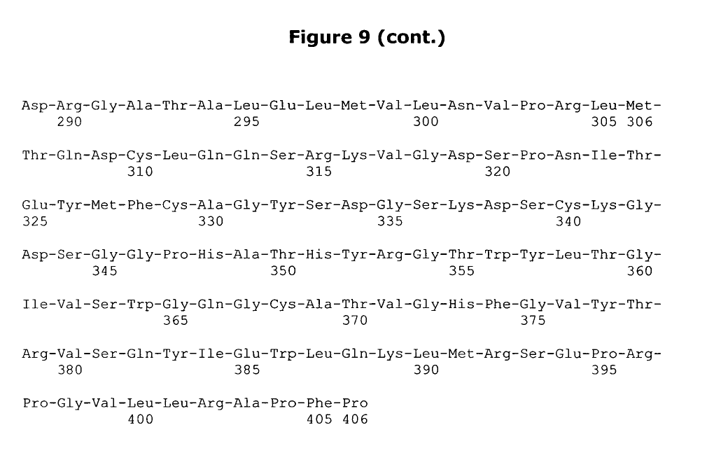

In one embodiment, the glycoprotein is FVII having the amino acid sequence of wild-type Factor VII (FIG. 9). In one embodiment, the polypeptides are wild-type Factor VIIa.

In one embodiment of the invention, the glycoprotein is a Factor VII polypeptide.

In one embodiment, the Factor VII polypeptide is a Factor VII variant having substantially the same biological activity as wild-type Factor VII including S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS) and WO 04/029091 (Maxygen ApS).

In one embodiment, the Factor VII polypeptide is a FVII variant having increased biological activity compared to wild-type FVIIa include FVII variants as disclosed in WO 01/83725, WO 02/22776, WO 02/077218, PCT/DK02/00635, Danish patent application PA 2002 01423, Danish patent application PA 2001 01627; WO 02/38162 (Scripps Research Institute); and FVIIa variants with enhanced activity as disclosed in JP 2001061479 (Chemo-Sero-Therapeutic Res Inst.).

In one embodiment, the Factor VII polypeptides are selected from the group consisting of: L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, S336G-FVII; Factor VII-sequence variants wherein the amino acid residue in positions 290 and/or 291, preferably 290, have been replaced; and Factor VII-sequence variants wherein the amino acid residue in positions 315 and/or 316, preferably 315, have been replaced. In another embodiment the Factor VII polypeptides are selected from the list consisting of: Factor VII variants having increased biological activity compared to wild-type FVIIa as disclosed in WO 01/83725, WO 02/22776, WO 02/77218, WO 03/27147, and WO 03/37932; L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A —FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; FVII having substitutions, additions or deletions in the amino acid sequence from 233Thr to 240Asn, FVII having substitutions, additions or deletions in the amino acid sequence from 304Arg to 329Cys, and FVII having substitutions, deletions, additions in the amino acid sequence Ile153-Arg223.

In one embodiment, the Factor VII-related polypeptides are selected from the group consisting of: R152E-Factor VII, S344A-Factor VII, FFR-Factor VII, and Factor VIIa lacking the Gla domain.

In one embodiment, the Factor VII-related polypeptide exhibit at least about 25%, preferably at least about 50%, more preferably at least about 75% and most preferably at least about 90% of the specific activity of wild-type Factor VIIa that has been produced in the same cell type, when tested in one or both of a hydrolysis assay or proteolysis assay as described in the present specification.

In one embodiment, the Factor VII-related polypeptide exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described in the present specification.

In different embodiments, the conjugated polypeptide exhibits a bioavailability that is at least about 110% of the bioavailability of a reference preparation, such as at least about 120%, or at least about 130%, or at least about 140% of the bioavailability of the reference preparation.

In one embodiment, the conjugated polypeptide exhibits a serum half-life that is at least about 125% of the half-life of a reference preparation, such as at least about 150%, or at least about 200%, or at least about 250% of the half-life of the reference preparation.

Functional Properties of Glycoprotein Conjugates

The glycoconjugates prepared according to the present invention exhibit improved functional properties relative to reference preparations. The improved functional properties may include, without limitation, a) physical properties such as, e.g., storage stability; b) pharmacokinetic properties such as, e.g., bioavailability and half-life; and c) immunogenicity in humans.

A reference preparation refers to a preparation comprising a polypeptide that has an amino acid sequence identical to that contained in the preparation of the invention to which it is being compared (such as, e.g., non-conjugated forms of wild-type Factor VII or a particular variant or chemically modified form) but which is not conjugated to any polymer molecule(s) found in the preparation of the invention. For example, reference preparations typically comprise non-conjugated glycoprotein.

Storage stability of a glycoprotein (such as, e.g., a Factor VII preparation) may be assessed by measuring (a) the time required for 20% of the bioactivity of a preparation to decay when stored as a dry powder at 25° C. and/or (b) the time required for a doubling in the proportion of predetermined degradation products, such as, e.g., aggregates, in the preparation.

In some embodiments, the preparations of the invention exhibit an increase of at least about 30%, preferably at least about 60% and more preferably at least about 100%, in the time required for 20% of the bioactivity to decay relative to the time required for the same phenomenon in a reference preparation, when both preparations are stored as dry powders at 25° C.

Bioactivity measurements may be performed in accordance with the kind of bioactivity associated with the particular protein; in case of, e.g., FVII, bioactivity may be measured using any of a clotting assay, proteolysis assay, TF-binding assay, or TF-independent thrombin generation assay.

In some embodiments, the preparations of the invention exhibit an increase of at least about 30%, preferably at least about 60%, and more preferably at least about 100%, in the time required for doubling of predetermined degradation products, such as, e.g., aggregates, relative to a reference preparation, when both preparations are stored as dry powders at 25° C. The content of aggregates may, for example, be determined by gel permeation HPLC, or another type of well-known chromatography methods. In case of FVII, aggregates may be determined by gel permeation HPLC on a Protein Pak 300 SW column (7.5×300 mm) (Waters, 80013) as follows. The column is equilibrated with Eluent A (0.2 M ammonium sulfate, 5% isopropanol, pH adjusted to 2.5 with phosphoric acid, and thereafter pH is adjusted to 7.0 with triethylamine), after which 25 μg of sample is applied to the column. Elution is with Eluent A at a flow rate of 0.5 ml/min for 30 min, and detection is achieved by measuring absorbance at 215 nm. The content of aggregates is calculated as the peak area of the Factor VII aggregates/total area of Factor VII peaks (monomer and aggregates).

“Bioavailability” refers to the proportion of an administered dose of a glycoconjugate that can be detected in plasma at predetermined times after administration. Typically, bioavailability is measured in test animals by administering a dose of between about 25-250 μg/kg of the preparation; obtaining plasma samples at predetermined times after administration; and determining the content of glycoprotein in the samples using a suitable bioassay, or immunoassay, or an equivalent. The data are typically displayed graphically as [glycoprotein] v. time and the bioavailability is expressed as the area under the curve (AUC). Relative bioavailability of a test preparation refers to the ratio between the AUC of the test preparation and that of the reference preparation.

In some embodiments, the preparations of the present invention exhibit a relative bioavailability of at least about 110%, preferably at least about 120%, more preferably at least about 130% and most preferably at least about 140% of the bioavailability of a reference preparation. The bioavailability may be measured in any mammalian species, preferably dogs, and the predetermined times used for calculating AUC may encompass different increments from 10 min-8 h.

Assays

Materials:

α2-3,6,8,9-Neuraminidase (E.C. 3.2.1.18) from Anthrobacter urefaciens was obtained from Calbiochem, Calif., USA, soluble neuraminidase from Vibreo cholerae, agarose supported neuraminidase from Clostridium perfringens and bovin catalase (E.C. 1.11.1.6) were obtained from Sigma-Aldrich. Galactose oxidase (E.C. 1.1.3.9) was obtained Worthington Biochemical Corporation, USA. PNGase F was from New England Biolabs Inc. MA, USA. All other chemicals were of standard grade and obtained from Sigma-Aldrich, Bachem or Fluka. Galactose oxidase kit A22179 was obtained from Molecular Probes, OR, USA. Proton and carbon nuclear magnetic resonance (1H and ¹³C NMR) were recorded on a Bruker 300 MHz or 400 MHz NMR apparatus, with chemical shift (δ) reported down field from tetramethylsilane. MALDI-TOF spectra were obtained using an Autoflex MALDI-TOF mass spectrophotometer from Bruker Daltonics Inc. Spectra were recorded in the linear mode using α-cyano-4-hydroxycinnamic acid as matrix.

Pharmacological Methods

The following assays are useful for determining biological activity, half-life and bioavailability of Factor VII and Factor VII-related polypeptides.

Assay (I)

In Vitro Hydrolysis Assay

The following method can be used to assay Factor VIIa bioactivity. The assay is carried out in a microtiter plate (MaxiSorp, Nunc, Denmark). The chromogenic substrate D-Ile-Pro-Arg-p-nitroanilide (S-2288, Chromogenix, Sweden), at a final concentration of 1 mM, is added to Factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used to calculate the ratio between the activities of a test and a reference Factor VIIa.

Assay (II)

In Vitro Proteolysis Assay

The following method can be used to assay Factor VIIa bioactivity. The assay is carried out in a microtiter plate (MaxiSorp, Nunc, Denmark). Factor VIIa (10 nM) and Factor X (0.8 uM) in 100 μl 50 mM Hepes, pH 7.4, containing 0.1 M NaCl, 5 mM CaCl₂ and 1 mg/ml bovine serum albumin, are incubated for 15 min. Factor X cleavage is then stopped by the addition of 50 μl 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of a test and a reference Factor VIIa.

Assay (III)

Measurement of Functional In Vivo Half-Life

Measurement of in vivo biological half-life can be carried out in a number of ways as described in the literature. An example of an assay for the measurement of in vivo half-life of rFVIIa and variants thereof is described in FDA reference number 96-0597. Briefly, FVIIa clotting activity is measured in plasma drawn prior to and during a 24-hour period after administration of the conjugate, polypeptide or composition. The median apparent volume of distribution at steady state is measured and the median clearance determined.

Assay (IV)

Bioavailability of Factor VII Polypeptides

Bioavailability may, for example, be measured in a dog model as follows: The experimentis performed as a four leg cross-over study in 12 Beagle dogs divided in four groups. All animals receive a test preparation A and a reference preparation B at a dose of about 90 μg/kg in a glycylglycine buffer (pH 5.5) containing sodium chloride (2.92 mg/ml), calcium chloride dihydrate (1.47 mg/ml), mannitol (30 mg/ml) and polysorbate 80. Blood samples are withdrawn at 10, 30, and 60 minutes and 2, 3, 4, 6 and 8 hours following the initial administration. Plasma is obtained from the samples and Factor VII is quantified by ELISA.

Analytical Methods:

CE-analysis: Capillary electrophoresis was performed on a Hewlett-Packard HP 3DCE system equipped with a UV-VIS DAD with a range from 200 to 600 nm and complete pneumatic system, enabling automated flushing of capillaries and replenishment of buffer vials. The instrument was operated in the normal polarity mode at +7.0-7.5 kV/+80-100 uA and detection was performed on column, at 214 nm with 280 nm as reference wavelength. CE analysis was carried out using fused-silica capillaries of 64,5 cm (55,5 cm effective length)×75 um I.D. The capillary was thermostated at 30° C. Samples were injected at the end of the capillary furthest away from the detector (long end injection) using +50 mbar for 10 s. The HP Chemstation software (Hewlett-Packard) operated on a HP Kayak XA computer (Hewlett-Packard) was used for instrument control, data acquisition and data analysis. Electrophoretic data were collected at a rate of 10 Hz. Electrolytes were prepared by dissolving known amount of putrescein dihydrochloride in 100 mM phosphate buffer (pH 8.0) and subsequently adjusting to pH 8.0 with 1N sodium hydroxide solutions (see N. K. Klausen and T. Kornfelt, J. Chromatography A, 718, 195-202 (1995)).

LCMS Analysis:

LC-MS mass spectra were obtained using apparatus consisting of a Hewlett Packard series 1100 G1312A Bin Pump, a Hewlett Packard series 1100 Column compartment, a Hewlett Packard series 1100 G13 15A DAD diode array detector and a Hewlett Packard series 1100 MSD. The instrument was controlled by HP Chemstation software. The HPLC pump was connected to two eluent reservoirs containing 0.01% TFA in water (A) and 0.01% TFA in acetonitril (B). The analysis was performed at 40° C. by injecting an appropriate volume of the sample (preferably 1 μL) onto the column, which was eluted with a gradient of acetonitrile. The HPLC conditions, detector settings and mass spectrometer settings used are as follows:

| | |
|---|---|
| Column | Waters Xterra MS C-18 × 3 mm id |
| Gradient | 10%-100% acetonitrile lineary during 7.5 min at 1.0 ml/min |
| Detection | UV: 210 nm (analog output from DAD) |
| MS | Ionisation mode: API-ES |
| | Scan 100-1000 amu step 0.1 amu |

Analysis of Natural and Modified N-Glycans:

The N-glycan analysis procedure was modified from D. I. Papac et al. Glycobiology 8(5), 445-454 (1998) and illustrated for FVIIa as followes: The pH of the FVIIa solution (1 mg/ml in 10 mM glycylglycin, 10 mM CaCl₂, 50 mM NaCl, pH 6.5) (50 μl) is adjusted to 8.3 by addition of NaOH 50 mM. PNGase F (500 U/ml in 10 mM Tris acetate buffer pH8.3) is added (20 μl, 10 U), followed by Tris acetate pH 8.3 buffer (total volume of the reaction mixture: 80 μl). The reaction mixture is incubated overnight at 37° C. PVDF membrane (Immobilon-P from Millipore, Millipore Corporation, Bedford, Ma, USA) (1.5 cm²) is added, the vial is shaken vigorously for 10 min, and the membrane is removed. Acetic acid 10% is added (10 μl) and the reaction mixture shaken for 2 h at RT. The reaction mixture is then added to the resin (Dowex 50W-X8, H form) (0.3 ml). Water (280 μl) is added, and the mixture is vortexed. The supernatant is taken out, evaporated, re-dissolved in water (20 μl), and analyzed by MALDI-TOF as described by Papac et al.

Determination of Exposed Galactose Residues:

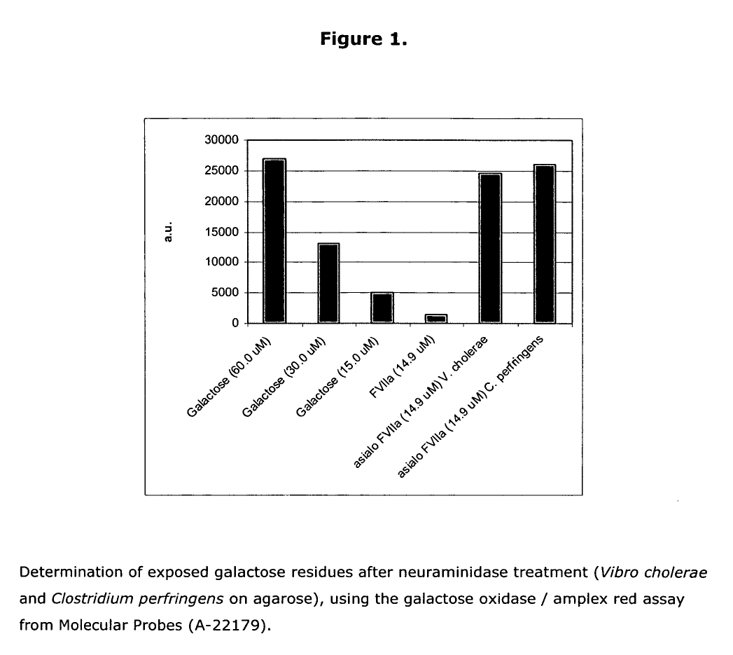

The amount of free (exposed) galactose residues present on the glycoprotein was determined using galactose assay kit A22179 and protocols from Molecular Probes. The assay was carried out in a microtiter plate format (MaxiSorp, Nunc, Denmark), and data was recorded on a Spectra Plus 384 plate reader (Molecular Device, USA), with exitation wave lenght 560 nm and emmision wave length 590 nm. FIG. 1 show a standard galactose oxidase row (60, 30 and 15 uM) and comparable results from 14.9 uM solution of FVIIa and asialo FVIIa respectively. A ratio of 3.70-3.90 mmol galactose/umol asialo FVIIa equivalent to complete removal of all sialic acids from FVIIa when assuming the presence of two N-linked (N145/322) bi-antenna structures.

### EXAMPLES

Example 1-4 illustrates the synthesis of representative O-substituted hydroxylamine nucleophiles suitable for conjugation to galactose oxidized glycans according to the invention.

### Example 1

### O-(N,N-Diethylaminoethyl)hydroxylamine

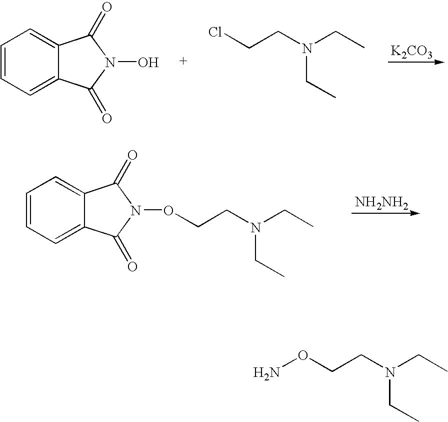

N-hydroxyphthalimide (10.0 g, 62.5 mmol) was dissolved in THF (100 ml) and potassium carbonate (18.63 g; 0.134 mol) was added. Diethylaminoethylchloride (10.54 g; 61.5 mmol) was added and the mixture was heated to reflux for 4 h. The mixture was cooled and added water (100 ml) and stirred until all solid had dissolved. The mixture was then extracted twice with DCM. The organic phase was dried with anhydrous sodium sulphate and evaporated to give 8.63 g (53%) of N-(N,N-diethylaminoethoxy)phthalimide as a clear yellow oil. The oil was dissolved in a minimum of acetonitril and 1.1 equivalent of 1N HCl in ethylacetate was added to precipitate N-(N,N-diethylaminoethoxy)phthalimide hydrochloride. ¹H-NMR (D₂O): δ 1.24 ppm (t, 6H); 3.28 (m, 4H); 3.56 (t, 2H); 4.45 (t, 2H); 7.72 (s, 4H). ¹³C-NMR (D₂O): δ 8.25 ppm; 47.93; 50.46; 72.10; 124.27; 128.34; 135.81; 165.82. N-(N,N-diethylaminoethoxy)phthalimide hydrochloride (2,50 g; 8.37 mmol) was dissolved in ethanol (20 ml) and hydrazine monohydrate (1 ml; 20.6 mmol) was added. The mixture was refluxed for 30 min. A precipitate quickly formed. The mixture was cooled on an ice bath. The crystalline material was removed by filtration, and the filtrate taken to dryness. A semi crystalline residue was obtained. This was re-suspended in cold ethanol (10 ml) and filtered. The filtrate was again taken to dryness, to yield 0.9 g of a clear yellow oil. ¹H-NMR (CDCl₃): δ 1.05 ppm (t, 6H); 2.58 (m, 4H); 2.65 (t, 2H); 3.75 (t, 2H); 5.12 (bs, 2H).

### Example 2

### 3-[(2-Aminooxyacetyl)-(2-carboxyethyl)amino]propionic acid

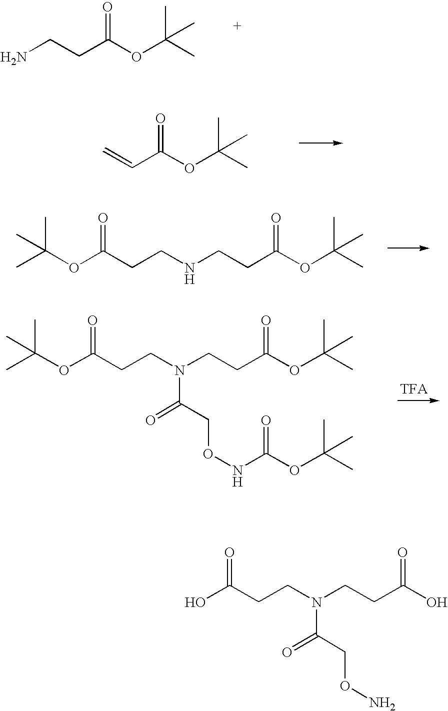

3-Aminopropionic acid tert-butyl ester hydrochloride (7.27 g. 40 mmol) was dissolved in DMF (40 ml). Triethylamine was added (4.05 g, 40 mmol). A solution of tert-butyl acrylate (5.13 g, 40 mmol) in DMF (40 ml) was added to the solution under inert atmosphere. After stirring at rt for 16 h, the precipitate was filtered off, and the filtrate is concentrated, dissolved in ethyl acetate (100 ml), washed with sat. NaHCO₃ (2×50 ml), dried over MgSO₄ and concentrated. The oil was purified by vacuum distillation (0.02 torr, 103-107° C.) to yield 3-(2-tert-butoxycarbonylethylamino)propionic acid tert-butyl ester as a colorless oil (4.7 g, 43% yield). ¹H-NMR (CDCl₃): δ 1.45 ppm (s, 18H), 2.41 (t, J=6.4 Hz, 4H), 2.84 (t, J=6.5 Hz, 4H). LCMS: m/z=274 (M+1), Rₜ=2.41 min.

3-(2-tert-Butoxycarbonylethylamino)propionic acid tert-butyl ester (0.63 g, 3.07 mmol), N-tert-butoxycarbonyl aminooxyacetic acid (1.07 g 5.27 mmol) and 1-hydroxybenzotriazole (0.50 g, 3.68 mmol) were dissolved in DMF (20 ml), and DIEA (0.48 g, 3.68 mmol) and (3-dimethylaminopropyl)-ethylcarbodiimide hydrochloride (0.71 g, 3.68 mmol) were added. The solution was stirred overnight under inert atmosphere. After concentrating the sample, it was dissolved in ethyl acetate (50 ml), washed with 5% acetic acid, sat. NaHCO₃ and water (2×20 ml each). The sample was dried over MgSO₄ and concentrated to a light yellow oil, 3-[(2-tert-butoxycarbonylaminooxyacetyl)-(2-tert-butoxycarbonylethyl)amino]propionic acid tert-butyl ester (1.05 g, 77% yield).

¹H-NMR (CDCl₃): δ 1.44 ppm (s, 18H), 1.47 (s, 9H), 2.47-2.57 (m, 4H), 3.48 (t, 1=7.1 Hz, 2H), 3.56 (t, J=7.1 Hz, 2H), 4.60 (s, 2H), 8.16 (s, 1H). LCMS: m/z=469 (M+23), Rₜ=4.30 min.

3-[(2-tert-butoxycarbonylaminooxyacetyl)-(2-tert-butoxycarbonylethyl)amino]propionic acid tert-butyl ester (0.50 g, 1.1 mmol) was stirred with trifluoroacetic acid (5 ml) under inert atmosphere for 1 h at rt. Conc. hydrochloric acid (2 ml) was added, and the sample was concentrated. The sample was dried by adding 10 ml toluene and concentrating the sample (twice). The residue was dissolved in ethanol (2 ml) and added drop wise to cold diethyl ether. The resulting precipitate adhered to the flask, and the solvent could be decanted off. The residue was dried under vacuum to yield a sticky white residue, 3-[(2-Aminooxyacetyl)-(2-carboxyethyl)amino]propionic acid. ¹H-NMR (D₂O): δ 2.67 ppm (t, J=6.8 Hz, 2H), 2.74 (t, J=6.8 Hz, 2H), 3.56 (t, J=6.8 Hz, 2H), 3.65 (t, J=7.2 Hz, 2H), 4.98 (s, 2H). LCMS: m/z=235 (M+1), Rₜ=0.37 min.

### Example 3

### mPEG₅₀₀₀-(CH₂)₂—CONH—(CH₂)₄—O—NH₂

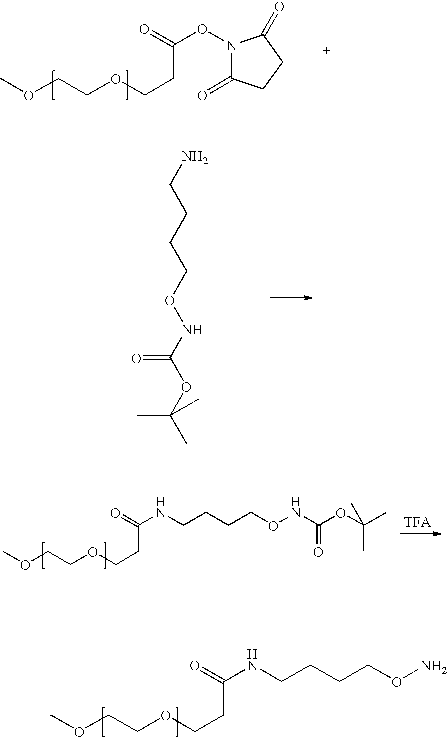

Step 1: O-(4-Aminobutyl)-N-(tert-butoxycarbonyl)hydroxylamine (120 mg, 0.584 mmol) was dissolved in DCM (25 ml) under nitrogen. mPEG₍₅₀₀₀₎-succinimidyl propionate (1 g, 0.195 mmol) was added, and the solution was stirred for 16 h. Diethyl ether (150 ml) was added, and the resulting precipitate was filtered off and washed with diethyl ether (2×60 ml). The precipitate was dried under vacuum for 16 h to yield a white solid (911 mg). mPEG₍₅₀₀₀₎-(CH₂)₂—CONH—(CH₂)₄—O—NH-Boc: ¹H-NMR (DMSO-d₆, 400 MHz): δ Among other signals was observed: 1.40 (s, 9H), 2.29 (t, 2H), 3.03 (m, 2H), 3.51 (s, 454H), 3.68 (m, 4H), 7.80 (t, 1H), 9.91 (s, 1H).

Step 2: mPEG₅₀₀₀-(CH₂)₂—CONH—(CH₂)₄—O—NH-Boc (911 mg, 0.174 mmol) was dissolved in TFA (8 ml). The solution was stirred under nitrogen for 30 min at rt. Diethyl ether (150 ml) was added, and after stirring for 30 min the resulting precipitate was filtered off and washed with diethyl ether (2×50 ml). The solid was dried in a vacuum oven at 35° C. for 16 h. to yield a white solid (800 mg, 90% yield).

mPEG₅₀₀₀-(CH₂)₂—CONH—(CH₂)₄—O—NH₂: ¹H-NMR (DMSO-d₆, 400 MHz): δ Among other signals was observed: 1.43 (m, 2H), 1.55 (m, 2H), 2.29 (t, 2H), 3.05 (m, 2H), 3.24 (s, 3H), 3.51 (s, 454H), 3.68 (t, 2H), 3.90 (t, 2H), 7.83 (t, 1H), 10.23 (br, 1H).

### Example 4

### 16-aminooxy-hexadecanoic acid

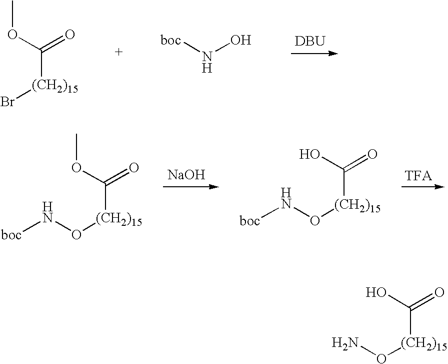

Step 1: 16-Bromo-hexadecanoic acid methyl ester (1.05 g, 3 mmol) and N-tert-butoxycarbonyl hydroxylamine (1 g, 7.5 mmol) were placed in a flask, and dissolved in DBU (2.25 ml). The solution was stirred under nitrogen for 3 h. DCM (100 ml) was added, and the solution was washed with 1 N HCl (3×25 ml) using some brine and methanol to assist in phase separation. The solution was dried over MgSO₄, and concentrated to yield an oily crystalline residue (1.09 g, 90% yield).

16-(N-tert-butoxycarbonylaminooxy)hexadecanoic acid methyl ester: ¹H-NMR (CDCl₃, 300 MHz): δ 1.25 (s, 20H, 1.48 (s, 9H), 1.62 (m, 4H), 2.30 (t, 2H), 3.67 (s, 3H), 3.84 (t, 2H), 7.16 (s,1H).

LCMS: m/z=425 (M+23), Rₜ=6.17.

Step 2: 16-(N-tert-butoxycarbonylaminooxy)hexadecanoic acid methyl ester (1.05 g, 2.62 mmol) was placed in THF (100 ml) and 1 N NaOH (2.75 ml) was added. The sample was stirred at rt for 16 h then refluxed for 2 h. After cooling to rt, 4N NaOH (20 ml) and methanol (50 ml) were added and the solution was stirred at rt for 45 min. The solution was neutralized with conc HCl and cooled in an ice bath. The precipitate was filtered off and the filtrate was concentrated under vacuum to yield a white solid, which was partitioned between DCM (100 ml) and 0.33 N HCl (150 ml) using 10 ml brine to assist in phase separation. The organic phase was dried over MgSO₄, and concentrated to yield an off-white residue (730 mg, 72% yield). The residue was purified via flash chromatography (silica, AcOEt/heptane 3:7 (400 ml) then 1:1 (200 ml)) to yield a white solid (150 mg, 15% yield). 16-(N-tert-butoxycarbonylaminooxy)hexadecanoic acid: ¹H-NMR (CDCl₃, 400 MHz): δ 1.25 (s, 20H), 1.49 (s, 9H), 1.62 (m, 4H), 2.35 (t, 2H), 3.84 (t, 2H), 7.23 (s, 1H). LCMS: m/z=410 (M+23), Rₜ=5.53.

Step 3: 16-(N-tert-butoxycarbonylaminooxy)hexadecanoic acid (110 mg, 0.28 mmol) was dissolved in TFA (2 ml) and stirred for 30 min at rt. Diethyl ether (20 ml) was added, and the precipitate was filtered off and dried under vacuum to yield a white solid (0.11 g).

16-aminooxyhexadecanoic acid: ¹H-NMR (DMSO-d₆, 400 MHz): δ 1.24 (s, 20H), 1.49 (t, 2H), 1.55 (t, 2H), 2.18 (t, 2H), 3.90 (t, 2H), 10.50 (br-m, 2H), 11.65 (br, 1H). LCMS: m/z=288 (M+1), Rₜ=3.58.

Example 5-15 illustrates enzymatic protocols and chemical conjugation methods for obtaining glycoproteins with modified glycan structures according to the invention.

Desialylation of rFVIIa (Preparation of Asialo rFVIIa)

### Example 5

Method Using Soluble Neuraminidase from Anthrobacter urefaciens.

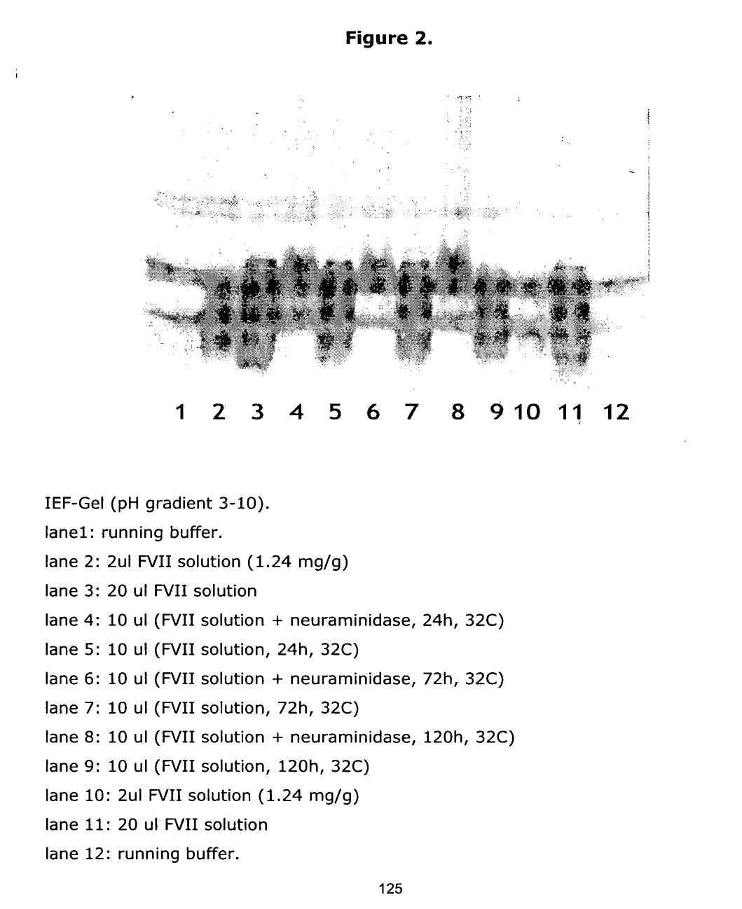

250 ul of a solution of rFVIIa (1.24 mg/ml, 24 uM) in a 10 mM glycylglycin buffer (pH 6.0) containing 10 mM CaCl₂ and 50 mM NaCl, was added 25 mU a2-3,6,8,9-neuraminidase, and the reaction mixture was incubated for 32 h at 4° C. To monitor the reaction, a small aliquot of the reaction was diluted with the appropriate buffer and an IEF gel performed according to Invitrogen's procedure (see FIG. 2). Samples were also analysed by MALDI-TOF spectroscopy using α-cyano-4-hydroxycinnamic acid matrix (see FIG. 3). An averaged loss in molecular weight of 700 Da was observed.

### Example 6

Method Using Soluble Neuraminidase from Vibro Cholerae.

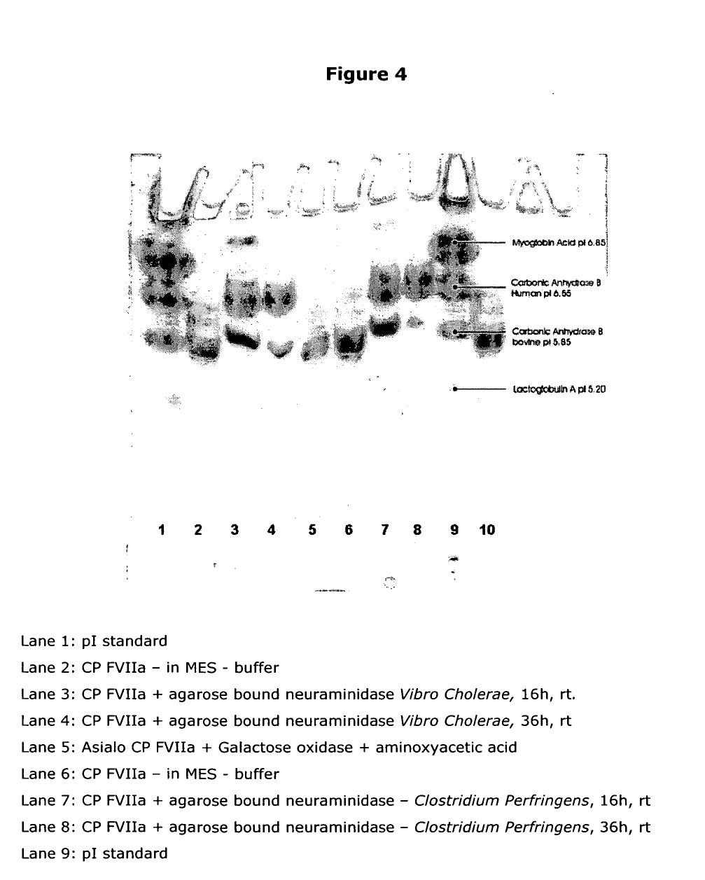

A 5 ml solution of FVIIa (1.4 mg/ml in 10 mM glycylglycin, 10 mM CaCl₂, 50 mM NaCl, pH 6), was added neuraminidase (20 ul, 300 mU, Vibro Cholerae, Type II, Sigma N 6514), and the mixture was shaken at room temperature for 24 h. The removal of sialic acids were confirmed by IEF-gel electrophoresis (Novex mini gel, pI 5.85->6.55, FIG. 4, lane 4) using the protocols devised by Invitrogen. The mixture was then cooled on ice, and pH was adjusted to 8.0 by addition of 50 mM aqueous NaOH. An aqueous solution of EDTA (450 ul, 100 mM, pH 8.0, equivalent to [Ca²⁺ ]) was added, and the sample (5.1 mS/cm) was loaded on a 5 ml HiTrap-Q HP ion-exchange column (Amersham-Biosciences), equilibrated with 10 mM Tris, 50 mM NaCl, pH 8.0. The column was eluted with 10 mM Tris, 50 mM NaCl, pH 8.0 (10 vol, flow: 1 ml/min). The elution buffer was then changed to 10 mM Tris, 50 mM NaCl, 25 mM CaCl₂, pH 8.0 (10 vol, flow: 1 ml/min). Pure FVIIa samples were pooled. The buffer was then ex-changed to 10 mM glycylglycin, 10 mM CaCl₂, 50 mM NaCl, pH 6 using NAP-10 columns (Amersham), and samples stored at −80° C. until later use. Amidolytic activity toward the peptide substrate S2288 (see in vitro hydrolysis assay section) was measuered to be identical to non-modified rFVIIa. The galactose assay kit from Molecular Probes (A22179), used to quantify the amount of exposed galactose residues on proteins (see the analytical procedure section), gave a ration of 3.70 mmol galactose/umol FVIIa (FIG. 1) equivalent to complete removal of all sialic acids from FVIIa when assuming the presence of two N-linked (N145/322) bi-antenna structures.

### Example 7

Method Using Agarose Supported Neuraminidase from Clostridium perfringens.

Neuraminidase-agarose resin (Clostridium perfringens, Type VI-A, Sigma N 5254) as ammonium sulfate suspension (2 ml), was washed extensively with MilliQ water, then drained and added to a 5 ml solution of FVIIa (1.4 mg/ml in 10 mM glycylglycin, 10 mM CaCl₂, 50 mM NaCl, pH 6). The mixture was shaken gently at room temperature for 48 h, then neuraminidase-agarose resin was filtered off. The removal of sialic acids were confirmed by IEF-gel electrophoresis (Novex mini gel, pI 5.85->6.55, FIG. 4, lane 8) using the protocols devised by Invitrogen. Amidolytic activity toward the peptide substrate S2288 (see in vitro hydrolysis assay section) was measuered to be identical to non-modified rFVIIa. The sample was stored at −80° C. until later use. The galactose assay kit from Molecular Probes (A22179), used to quantify the amount of exposed galactose residues on proteins (see the analytical procedure section), gave a ratio of 3.90 mmol galactose/umol FVIIa (FIG. 1) equivalent to complete removal of all sialic acids from FVIIa when assuming the presence of two N-linked (N145/322) bi-antenna structures. Removal of sialic acids can alternatively by analyzed by the CE-protocol as described in the analytical section.

### Example 8

General one-step procedure for preperation of galactosyl derivatized FVIIa analogues: Step A (buffer eschange): FVIIa (1 ml, 28 nmol, 1.4 mg/ml) in 10 mM glycylglycin buffer (pH 6.0) containing 10 mM CaCl₂ and 50 mM NaCl is added to a 1 ml NAP-10 column (Amersham Bioscience) previously callibrated with 15 ml of 10 mM MES buffer (pH 6.0) containing 10 mM CaCl₂ and 50 mM NaCl. The solution is allowed to pass into the bed of the column. Then 1.5 ml of 10 mM MES buffer (pH 6.0) containing 10 mM CaCl₂ and 50 mM NaCl is added, while collecting the 1.5 ml of eluate.

Step B (neuraminidase treatment): The eluate is added 75 mU neuraminidase (from Vibro cholerae, EC 3.2.1.18), and the mixture is incubated at room temperature for 24 h.

Step C (galactose oxidase+reaction with nucleophile): A freshly prepared solution of 12 U galactose oxidase (EC. 1.1.3.9) and 240 U of catalase (EC. 1.11.1.6) and O-substituted hydroxylamine derivative (500 nmol or approximately 5 equivalents per oxidized galactose residue) in 200 ul MES buffer (10 mM MES, 10 mM CaCl₂ and 50 mM NaCl, pH 6.0) is added. Extent of modification is followed by analytical techniques suitable for the particular modification in question (e.g. IEF gels for charged moieties or reducing/non-reducing SDS-PAGE gels for hydroxylamines of larger size), but between 8-24 hr at room temperature is typical for obtaining conversion. N-glycanes from a small aliquot are subsequently cleaved using the PNGase F protocol and analysed using procedures described in the analytical section, and the FVIIa modified end product is then purified as described in example 6 and stored at −80° C. until later use.

### Example 9

General Procedure for One-Pot Galactose Oxidation of Asialo rFVIIa and Reaction with Hydroxylamines (R—O—NH₂):

A solution of 250 ul desialylated rFVIIa (24 uM) in a 10 mM MES buffer (pH 6.0) containing 10 mM CaCl₂ and 50 mM NaCl (prepared as described above) is reacted with 930 mU of galactose oxidase and catalase (145 U) in 10 mM MES buffer (pH 6.0) containing 1.20 mM (50×) of O-derivatized hydroxylamine. The reaction is placed at 4° C. for 24-72 h. The progress of the reaction is monitored either by capillary electrophoresis using the method specified in the material section, or by performing an IEF gel (or SDS-PAGE gel depending on the type of hydroxylamine) on small aliquots according to Invitrogen's procedure. When the reaction has reached completion, the glycan derivatized FVIIa analogue is purified by ion-exchange chromatography according to L. Thim et al. Biochemistry, 1988, 27, 7785-7793, or as described in example 6. The product is finally characterized by capillary electrophoreses as described in the analytical section, MALDI-TOF or other appropriate methods for protein analysis. Modified N-glycans are cleaved using the PNGase F protocol and analyzed by MALDI-TOF method described in the analytical section.

### Example 10

General Procedure for One-Pot Galactose Oxidation of an Asialo Glycoprotein and Reaction with Nucleophilic Agents (e.g. R—CO—NHNH2, R—NHNH2 or R—O—NH₂):

A solution of 250 ul desialylated glycoprotein (24 uM) in a 10 mM MES buffer (pH 6.0) is reacted with galactose oxidase and catalase in 10 mM MES buffer (pH 6.0) containing 1.20 mM (50×) of nucleophilic agent (e.g. R—CO—NHNH2, R—NHNH2 or R—O—NH₂). The reaction is placed at 4° C. for 24-72 h. The progress of the reaction is monitored either by capillary electrophoresis using the method specified in the material section, or by performing an IEF gel (or SDS-PAGE gel depending on the type of hydroxylamine) on small aliquots according to Invitrogen's procedure. When the reaction has reached completion, the glycan derivatized glycoprotein is purified by appropriate chromatographically techniques. The product is finally characterized by capillary electrophoreses, MALDI-TOF or other appropriate methods for protein analysis. Modified N-glycans is analysed by the PNGase F-MALDI-TOF method described in the analytical section.

### Example 11

General Procedure for One-Pot Galactose Oxidation of an Asialo Glycoprotein and Reaction with Nucleophilic Agents (e.g. R—CO—NHNH2, R—NHNH2 or R—O—NH₂) Using Bead Supported Enzymes:

A solution of 250 ul desialylated glycoprotein (24 uM) in a 10 mM MES buffer (pH 6.0) is reacted with agarose immobilised galactose oxidase (100 mg, with a typically activity of 70 U/g support) and agarose immobilised catalase (100 mg, with a typically activity of 3000 U/g support) in MES buffer (pH 6.0) containing 1.20 mM (50×) of nucleophilic agent (e.g. R—CO—NHNH2, R—NHNH2 or R—O—NH₂). The reaction is placed at 4° C. for 24-72 h. The progress of the reaction is monitored either by capillary electrophoresis using the method specified in the material section, or by performing an IEF gel (or SDS-PAGE gel depending on the type of hydroxylamine) on small aliquots according to Invitrogen's procedure. When the reaction has reached completion, bead supported enzymes are removed by filtration and the glycan derivatized glycoprotein subsequently purified by appropriate chromatographically techniques. The product is finally characterized by capillary electrophoreses, MALDI-TOF or other appropriate methods for protein analysis. Modified N-glycans is analysed by the PNGase F-MALDI-TOF method described in the analytical section.

### Example 12

mPEG₅₀₀₀-(CH₂)₂—CONH—(CH₂)₄—O—NH₂ Ligation to FVIIa.

Step 1 (preparation of asialo FVIIa): Neuraminidase (Clostridium Perfringens on agarose, Sigma N5254) was washed with milli-Q water (3×15 ml), and was added to a solution of FVIIa (11 ml, 1.4 mg/ml, in Gly-gly buffer). The sample was shaken gently for 16 h. The neuraminidase was filtered off, and the buffer was exchanged to a MES buffer (10 mM MES, 10 mM CaCl₂, 50 mM NaCl, pH 6) using NAP-25 columns and a NAP-10 column (Amersham biosciences), yielding the asialo FVIIa in a MES buffer. Analysis using an IEF-gel indicates a change in the proteins pI.

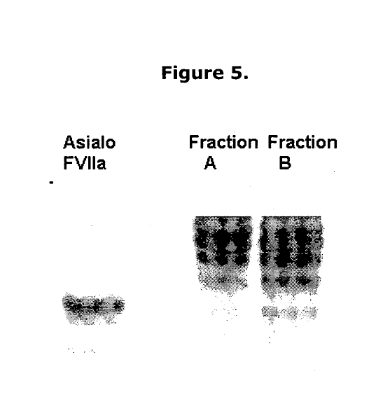

Step 2 (One-pot Galactose oxidation and oxime formation with mPEG₍₅₀₀₀₎-(CH₂)₂—CONH—(CH₂)₄—O—NH₂): Some of the asialo FVIIa from above (5 ml) was added Galactose oxidase (1.28 mg, of 51 U/mg), Catalase (9.24 mg of 1183 U/mg) and mPEG₍₅₀₀₀₎-(CH₂)₂—CONH—(CH₂)₄—O—NH₂ (9.53 mg). The sample was allowed to stand at rt for 20 h. The pH was adjusted to 8 with 50 mM NaOH, and 100 mM EDTA (500 μl) was added, after which the pH was again adjusted to 8. The sample was purified using a 5 ml Hi-Trap Q column (Amersham Biosciences), Start buffer (10 mM TRIS, 50 mM NaCl, pH 8.0), and eluting buffer (10 mM TRIS, 50 mM NaCl, 25 mM CaCl₂, pH 8.0). The fractions containing FVIIa were collected and analysed by SDS-PAGE. The increased mass of the products was visible by SDS-PAGE (FIG. 5)

### Example 13

Large Scale Method for Preparing Aminoxyacetic Acid Derivatized FVIIa.

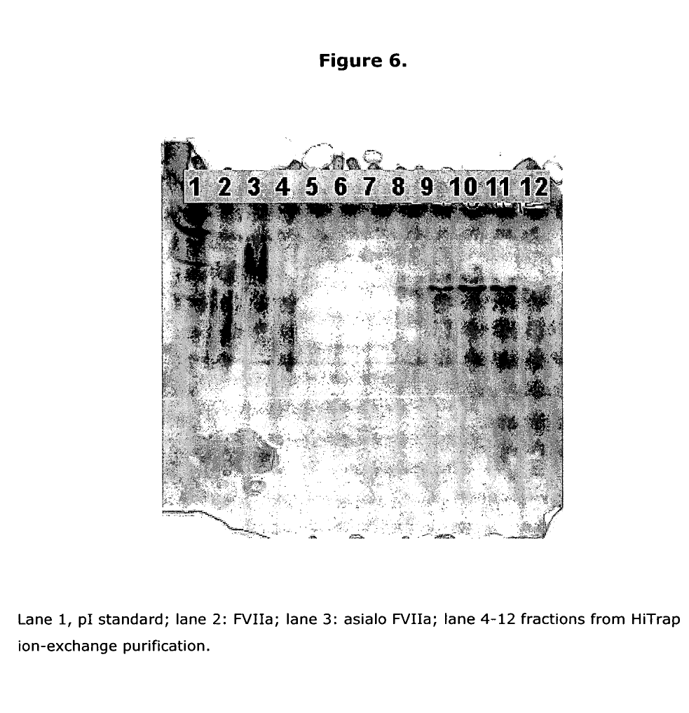

Asialo FVIIa (10.5 mg, in Gly-Gly buffer pH 6.0 (7.5 ml)), as prepared in example 2 or 3 was submitted to buffer exchanged to a 10 mM MES, 10 mM CaCl₂, 50 mM NaCl, pH 6.0 buffer, using three NAP-25 (Amersham) column previously equilibrated with 10 mM MES, 10 mM CaCl₂, 50 mM NaCl, pH 6.0. Then 2.25 ml of a 10 mM aminoxyaceticacid solution in 10 mM MES, 10 mM CaCl₂, 50 mM NaCl, pH 6.0 was added, followed by 135 U galactose oxidase (1.93 mg, 78 U/mg) and 6039 U catalase (2.57 mg, 2350 U/mg) in 2.25 ml 10 mM MES, 10 mM CaCl₂, 50 mM NaCl, pH 6.0. The mixture was incubated at 4° C. for 48 h with occational shaking. pH was raised to 8.0 using 50 mM NaOH. Calcium ions were removed from solution by addition of 100 mM EDTA solution at pH 8.0 (1.7 ml). The conductivity of the solution was measured to 8.3 mS/cm. The solution was then loaded on a 5 ml HiTrap-Q HP ion-exchange column (Amersham-Biosciences), equilibrated with 10 mM Tris, 50 mM NaCl, pH 8.0. The column was eluted with 10 mM Tris, 50 mM NaCl, pH 8.0 (10 vol, flow: 1 ml/min). The elution buffer was then changed to 10 mM Tris, 50 mM NaCl, 25 mM CaCl₂, pH 8.0 (10 vol, flow: 1 ml/min). Fractions were analyzed by IEF gels (FIG. 6) and pure aminoxyacetic acid modified FVIIa samples (having pI of approximatly 5.8 on the gel) were pooled. The buffer was then ex-changed to 10 mM Gly-Gly, 10 mM CaCl₂, 50 mM NaCl, pH 7 using NAP-10 columns (Amersham), and samples stored at −80° C. until later use. The peptidolytical activity using the S2288 peptide substrate (see the in vitro assay section) was measured to 54% of starting material.

### Example 14

One Pot Method for Derivatization with p-Nitrobenzyloxyamine. Step a (Buffer Exchange):

FVIIa (1 ml, 28 nmol, 1.4 mg/ml) in 10 mM GlyGly buffer (pH 6.0) containing 10 mM CaCl₂ and 50 mM NaCl was added to a 1 ml NAP-10 column (Amersham Bioscience) previously callibrated with 15 ml of 10 mM MES buffer (pH 6.0) containing 10 mM CaCl₂ and 50 mM NaCl. The solution was allowed to pass into the bed of the column. Then 1.5 ml of 10 mM MES buffer (pH 6.0) containing 10 mM CaCl₂ and 50 mM NaCl was added, while collecting the 1.5 ml of eluate.

Step B (neuraminidase treatment): The eluate was added 75 mU neuraminidase (from Vibro cholerae, EC 3.2.1.18), and the mixture was incubated at room temperature for 24 h.

Step C (galactose oxidase+reaction with nucleophile): A freshly prepared solution of 12 U galactose oxidase (EC. 1.1.3.9) and 240 U of catalase (EC. 1.11.1.6) and p-nitrobenzyloxyamine (500 nmol or approximately 5 equivalents per oxidized galactose residue) in 200 ul MES buffer (10 mM MES, 10 mM CaCl₂ and 50 mM NaCl, pH 6.0) was added. The mixture was allowed to react for 24 h at rt.

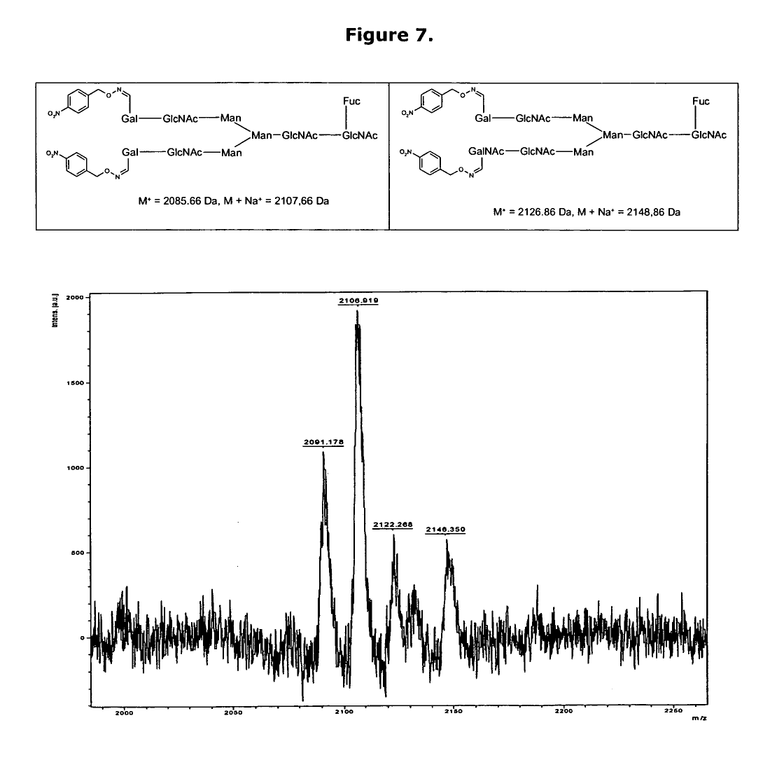

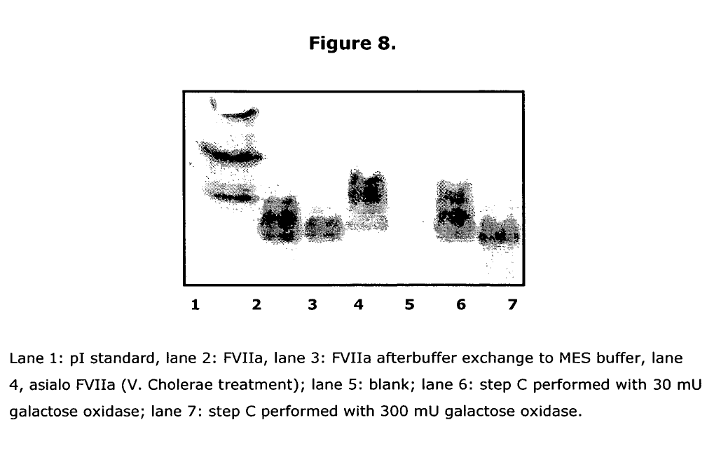

Step D (analysis of derivatized N-glycanes): All the sample was used for analysis. The N-glycanes were cleaved using PNGase F, and the released N-glycanes identified by MALDI-TOF spectroscopy using the methods described in the analytical section. Two derivatized glycan structures was identified (FIG. 7) corresponding to modified biantenna with Gal and GalNAc modifications respectively.

### Example 15

One-Pot Derivatization with Aminoxyacetic Acid.

Step A (buffer exchange): FVIIa (1 ml, 28 nmol, 1.4 mg/ml) in 10 mM GlyGly buffer (pH 6.0) containing 10 mM CaCl₂ and 50 mM NaCl was added to a 1 ml NAP-10 column (Amersham Bioscience) previously callibrated with 15 ml of 10 mM MES buffer (pH 6.0) containing 10 mM CaCl₂ and 50 mM NaCl. The solution was allowed to pass into the bed of the column. Then 1.5 ml of 10 mM MES buffer (pH 6.0) containing 10 mM CaCl₂ and 50 mM NaCl was added, while collecting the 1.5 ml of eluate.

Step B (neuraminidase treatment): 1 ml of the eluate was added 150 mU neuraminidase (from Vibro cholerae, EC 3.2.1.18), and the mixture was incubated at room temperature for 24 h.

Step C (galactose oxidase+reaction with nucleophile): A freshly prepared solution of 300 mU galactose oxidase (EC. 1.1.3.9) and 1316 U of catalase (EC. 1.11.1.6) and aminoxyacetic acid hemi chloride (500 nmol or approximately 5 equivalents per oxidized galactose residue) in 200 ul MES buffer (10 mM MES, 10 mM CaCl₂ and 50 mM NaCl, pH 6.0) was added. The mixture was allowed to react for 24 h at rt. Aliquotes taken from each step was analysed by IEF-gel analysis (FIG. 8). Step C was also carried out using 30 mU galactose oxidase.

### Example 16

Desialylation of Sialylated Glycoproteins Using Solid Supported Neuraminidases (Preparation of Asialo Glycoproteins):

250 ul of a solution of a sialylated glycoprotein (1.24 mg/ml, 24 uM) in a 10 mM MES buffer (pH 6.0) is added a2-3,6,8,9-neuraminidase immobilised on agarose support and the reaction mixture is incubated for 32 h at 4° C. To monitor the reaction, a small aliquot of the reaction is diluted with the appropriate buffer and an IEF gel performed according to Invitrogen's procedure. Samples is also analysed by capillary electrophoresis according to the methods described in the material section.

### Example 17

The Following Examples are Relevant for the Synthesis of Dendrimer Compounds.

Examples and general procedures refer to intermediate compounds and final products identified in the structural specification and in the synthesis schemes. The preparation of the dendrimer compounds of the present invention is described in detail using the following examples, but the chemical reactions described are disclosed in terms of their general applicability to the preparation of selected branched polymers of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents. All reagents were of standard grade as supplied from Aldrich, Sigma, ect. Proton, carbon and phosphor nuclear magnetic resonance (¹H-, ¹³C- and ³¹P-NMR) were recorded on a Bruker NMR apparatus, with chemical shift (6) reported down field from tetramethylsilane or phosphoric acid. LC-MS mass spectra were obtained using apparatus and setup conditions as follows:
Hewlett Packard series 1100 G1312A Bin Pump
Hewlett Packard series 1100 Column compartment
Hewlett Packard series 1100 G13 15A DAD diode array detector
Hewlett Packard series 1100 MSD
   The instrument was controlled by HP Chemstation software.
   The HPLC Pump was Connected to Two Eluent Reservoirs Containing:

A: 0.01% TFA in water

B: 0.01% TFA in acetonitrile

The analysis was performed at 40° C. by injecting an appropriate volume of the sample (preferably 1 μL) onto the column, which was eluted with a gradient of acetonitrile. The HPLC conditions, detector settings and mass spectrometer settings used are given in the following table.

| | |
|---|---|
| Column | Waters Xterra MS C-18, 5 um, 50 × 3 mm id |
| Gradient | 10%-100% acetonitrile lineary during 7.5 min at 1.0 ml/min |
| Detection | UV: 210 nm (analog output from DAD) |
| MS | Ionisation mode: API-ES |
| | Scan 100-1000 amu step 0.1 amu |

Some of the NMR data shown in the following examples are only selected data. In the examples the following terms are intended to have the following, general meanings:

Abbreviations

Boc: tert-butoxycarbonyl
CDI: carbonyldiimidazole
DCM: dichloromethane, methylenechloride
DIC: diisopropylcarbodiimide
DIPEA: N,N-diisopropylethylamine
DhbtOH: 3-hydroxy-1,2,3-benzotriazin-4(3H)-one
DMAP: 4-dimethylaminopyridine
DMF: N,N-dimethylformamide
DMSO: dimethyl sulphoxide
DTT: Dithiothreitol
EtOH: ethanol
Fmoc: 9-fluorenylmethyloxycarbonyl
HOBt: 1-hydroxybenzotriazole
MeOH: methanol
NMP: N-methyl-2-pyrrolidinone
NEt₃: triethylamine
THF: tetrahydrofuran
TFA: trifluoroacetic acid
TSTU: 2-succinimido-1,1,3,3-tetramethyluronium tetrafluoroborate

The following non limiting examples illustrates the synthesis of monomers and polymerisation technique using solid phase synthesis or solution phase synthesis.

Synthesis of Monomer Building Blocks and Linkers

### Example 17

### 2-[2-(2-Chloroethoxy)ethoxymethyl]oxirane

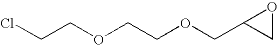

2-(2-Chloroethoxy)ethanol (100.00 g; 0.802 mol) was dissolved in dichloromethane (100 ml) and a catalytical amount of boron trifluride etherate (2.28 g; 16 mmol). The clear solution was cooled to 0° C., and epibromhydrin (104.46 g; 0.762 mol) was added dropwise maintaining the temperature at 0° C. The clear solution was stirred for an additional 3 h at 0° C., then solvent was removed by rotary evaporation. The residual oil was evapoprated once from acetonitrile, to give crude 1-bromo-3-[2-(2-chloroethoxy)ethoxy]propan-2-ol, which was re-dissolved in THF (500 ml). Powdered potassium tert-butoxide (85.0 g; 0.765 mmol) was then added, and the mixture was heated to reflux for 30 min. Insoluble salts were removed by filtration, and the filtrate was concentrated, in vacuo, to give a clear yellow oil. The oil was further purified by vacuum destillation, to give 56.13 g (41%) of pure title material.

bp=65-75° C. (0.65 mbar). ¹H-NMR (CDCl₃): δ2.61 ppm (m, 1H); 2.70 (m, 1H); 3.17 (m, 1H); 3.43 (dd, 1H); 3.60-3.85 (m, 9H). ¹³C-NMR (CDCl₃): δ42.73 ppm; 44.18; 50.80; 70.64 & 70,69 (may collaps); 71.37; 72.65.

### Example 18

### 1,3-Bis[2-(2-chloroethoxy)ethoxy]propan-2-ol

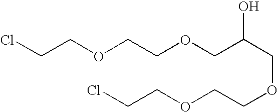

2-[2-(2-Chloroethoxy)ethoxymethyl]oxirane (2.20 g; 12.2 mmol) was dissolved in DCM (20 ml), and 2-(2-chloroethoxy)ethanol (1.52 g; 12.2 mol) was added. The mixture was cooled to 0° C. and a catalytical amount of boron trifluride etherate (0.2 ml; 1.5 mmol) was added. The mixture was stirred at 0° C. for 2 h, then solvent was removed by rotary evaporation. Residual of boron trifluride etherate was removed by co-evaporating twice from acetonitril. The oil thus obtained was purified by kuglerohr destilation. The title material was obtained as a clear viscous oil in 2.10 g (45%) yield. bp.=270° C., 0.25 mbar. ¹H-NMR (CDCl₃): δ3.31 (bs, 1H); 3.55 ppm (ddd, 4H); 3.65-3.72 (m, 12H); 3.75 (t, 4H); 3.90 (m, 1H). ¹³C-NMR (CDCl₃): 643.12 ppm; 69.92; 70.95; 71.11; 71.69; 72.69.

### Example 19

### 1,3-Bis[2-(2-azidoethoxy)ethoxy]propan-2-ol

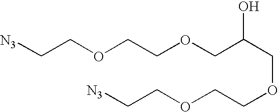

1,3-Bis[2-(2-chloroethoxy)ethoxy]propan-2-ol (250 mg; 0.81 mmol) was dissolved in DMF (2.5 ml), and sodium azide (200 mg; 3.10 mmol) and sodium iodide (100 mg; 0.66 mmol) were added. The suspension was heated to 100° C. (internal temperature) over night. The mixture was then cooled and filtered. The filtrate was taken to dryness, and the semi crystalline oil resuspended in DCM (5 ml). The non-soluble salts were removed by filtration; the filtrate was evaporated to dryness to give pure title mateial as a colorless oil. Yield: 210 mg (84%). ¹H-NMR (CDCl₃): δ3.48 ppm (t, 4H); 3.60-3.75 (m, 16H); 4.08 (m, 1H). ¹³C-NMR (CDCl₃): δ51.05 ppm; 69.10; 70.24; 70.53; 70.78; 71.37. LC-MS (any-one): m/e=319 (M+1)⁺; 341 (M+Na)⁺; 291 (M-N₂)⁺. Rₜ=2.78 min.

### Example 20

### 1,3-Bis[2-(2-azidoethoxy)ethoxy]propan-2-yl-p-nitrophenylcarbonate

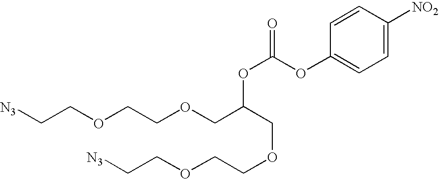

1,3-Bis[2-(2-azidoethoxy)ethoxy]propan-2-ol (2.00 g; 6.6 mmol) was dissolved in THF (50 ml) and diisopropylethylamine (10 ml) was added. The clear yellow solution was then added 4-dimethylaminopyridine (1.60 g; 13.1 mmol) and p-nitorphenylchloroformiate (2.64 g; 13.1 mmol) and stirred at ambient temperature. A precipitate rapidly formed. The suspension was stirred for 5 h at room temperature, then filtered and concentrated in vacuo. The residue was further purified by chromatography using ethylacetate-heptane-triethylamine (40/60/2) as eluent. The product was obtained as a clear yellow oil in 500 mg (16%) yield. ¹H-NMR (CDCl₃): δ 3.38 ppm (t, 4H); 3.60-3.72 (m, 12H); 3.76 (m, 4H); 5.12 (q, 1H); 7.41 (d, 2H); 8.28 (d, 2H). LC-MS (any-one): m/e=506 (M+Na)⁺; 456 (M-N₂)⁺. Rₜ=4.41 min.

### Example 21

### 1,3-Bis[2-(2-azidoethoxy)ethoxy]propan-2-yl chloroformiate

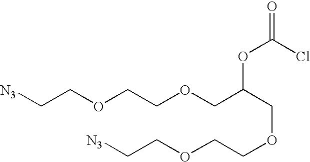

Trichloroacetylchloride (1,42 g, 7.85 mmol) was dissolved in THF (10 ml), and the solution was cooled to 0° C. A solution of 1,3-Bis[2-(2-azidoethoxy)ethoxy]propan-2-ol (1.00 g; 3.3 mmol) and triethylamine (0,32 g, 3.3 mmol) in THF (5 ml) was slowly added drop wise over 10 min. Cooling was removed, and the resulting suspension was stirred for 6 h at ambient temperature. The mixture was filtered, and the filtrate was evaporated to give a light brown oil. The oil was treated twice with acetonitril following evaporation, and the product was used without further purification.

¹H-NMR (CDCl₃): δ 3.40 (t, 4H); 3,55-3,71 (m, 12H); 3,75 (d, 4H); 5.28 (m, 1H).

### Example 22

### 2-(1,3-Bis[2-(2-azidoethoxy)ethoxy]propan-2-yloxy)acetic acid

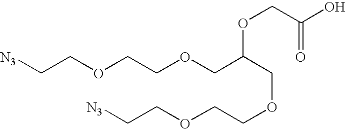

Sodium hydride (7.50 g; 80% oil suspension) was washed trice with heptanes, and then re-suspended in dry THF (100 ml). A solution of 1,3-bis[2-(2-azidoethoxy)ethoxy]propan-2-ol (10.00 g; 33.0 mmol) in dry THF (100 ml) was then slowly added over a period of 30 min at room temperature. Then a solution of bromo acetic acid (6.50 mg; 47 mmol) in THF (100 ml) was added drop wise over 20 min.->slight heat evolution. A cream coloured suspension was formed. The mixture was stirred at ambient temperature over night. Excess sodium hydride was carefully destroyed by addition of water (20 ml) while cooling the mixture. The suspension was taken to dryness by rotary evaporation, and the residue partitioned between DCM and water. The water phase was extracted twice with DCM then acidified by addition of acetic acid (25 ml). The water phase was then extracted twice with DCM, and the combined organic phases were dried over sodium sulphate, and evaporated to dryness. The residual oil at this point contained the title material as well as bromo acetic acid. The later was removed by re-dissolving the oil in DCM (50 ml) containing piperidine (5 ml); stir for 30 min., and then wash the organic solution trice with 1N aquoeus HCl (3×). Pure title material was then obtained after drying (Na2SO4) and evaporation of the solvent. Yield: 7.54 g (63%).

¹H-NMR (CDCl₃): δ 3.48 ppm (t, 4H); 3.55-3.80 (m, 16H); 4.28 (s, 2H); 4.30 (m, 1H); 8.50 (bs, 1H). ¹³C-NMR (CDCl₃): δ 51.04 ppm; 69.24; 70.50; 70.72; 71.39; 71.57; 80.76; 172.68. LC-MS (any-one): m/e=399 (M+Na)⁺; 349 (M-N₂)⁺. Rₜ=2.34 min.

### Example 23

### Imidazole-1-carboxylic acid 1,3-bis(2-(2-azidoethoxy)ethoxy)propan-2-yl ester

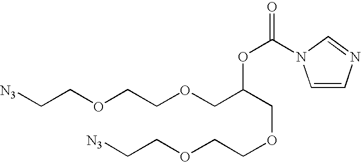

1,3-Bis[2-(2-azidoethoxy)ethoxy]propan-2-ol (1.00 g; 3.3 mmol) was dissolved in DCM (5 ml) and carbonyl diimidazole (1.18 g, 6.3 mmol) was added. The mixture was stirred for 2 h at room temperature. Solvent was removed and the residue was dissolved in methanol (20 ml) and stirred for 20 min. Solvent was removed and the clear oil, thus obtained was further purified by column chromatography on silica using 2% MeOH in DCM as eluent. Yield: 372.4 mg (35%). ¹H-NMR (CDCl₃): δ 3.33 (t, 4H); 3,60-3,75 (m, 12H); 3,80 (d, 4H); 5.35 (m, 1H); 7.06 (s, 1H); 7.43 (s, 1H); 8.16 (s, 1H). LC-MS (any-one): m/e=413 (M+1)⁺; Rₜ=2.35 min.

### Example 24

### t-Butyl 2-(1,3-bis[2-(2-azidoethoxy)ethoxy]propan-2-yloxy)acetate

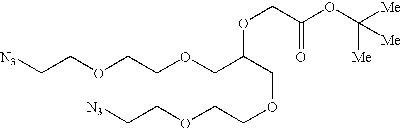

2-(1,3-Bis[2-(2-azidoethoxy)ethoxy]propan-2-yloxy)acetic acid (5.0 g; 13.28 mmol) was dissolved in toluene (20 ml), and the reaction mixture was heated to reflux under an inert atmosphere. N,N-dimethylformamid-di-tert-butylacetal (13 ml; 54.21 mmol) was then added dropwise over 30 min. Reflux was continued for 24 h. The dark brown solution was then filtered through Celite. Solvent was removed under vacuum, and the oily residue was purified by flash chromatography on silica, using 3% methanol dichloromethane as eluent. Pure fractions were pooled and evaporated to dryness. The title material was obtained as a yellow clear oil. Yield: 5.07 g (88%). ¹H-NMR (CDCl₃): δ 1.42 ppm (s, 9H); 3.35 (t, 4H); 3.54-3.69 (m, 16H); 3.75-3.85 (m, 1H); 4.16 (s, 2H). ¹³C-NMR (CDCl₃, selected peaks): δ 30.35 ppm.; 52.93; 70.65; 72.25; 73.12; 73.90; 80.44; 83.55; 172.28. R_{f}=0.33 in ethyl acetate-heptane (1:1).

### Example 25

### t-Butyl 2-(1,3-bis[2-(2-aminoethoxy)ethoxy]propan-2-yloxy)acetate

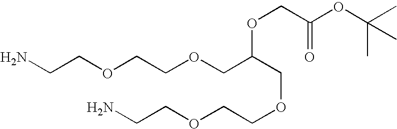

t-Butyl 2-(1,3-bis[2-(2-azidoethoxy)ethoxy]propan-2-yloxy)acetate (5.97 g, 11.7 mmol) was dissolved in ethanol-water (25 ml; 2:1), and acetic acid (5 ml) was added, followed by a aqueous suspension of Raney-Nickel (5 ml). The mixture was then hydrogenated at 3 atm., for 16 h using a Parr apparatus. The catalyst was then removed by filtration, and the reaction mixture was taken to dryness by rotary evaporation. The oily residue was dissolved in water and freeze dried to give a quantitative yield of title material. ¹H-NMR (CDCl₃): δ 1.45 ppm (s, 9H); 3.15 (bs, 4H); 3.48-3.89 (broad m, 17H); 4.15 (s, 2H). ¹³C-NMR (CDCl₃, selected peaks): δ 28.44 ppm.; 39.81; 68.17; 70.58; 70.79; 70.99; 78.81; 82.31; 170.59.

### Example 26

### 2-(1,3-Bis[2-(2-aminoethoxy)ethoxy]propan-2-yloxy)acetic acid

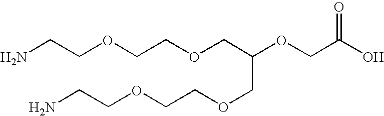

2-(1,3-Bis[2-(2-azidoethoxy)ethoxy]propan-2-yloxy)acetic acid (1.00 g; 2.65 mmol) was dissolved in 1N aqueous hydrochloric acid (10 ml) and a 50% aqueous suspension of 5% palladium on carbon (1 ml) was added. The mixture was hydrogenated at 3.5 atm using a Parr apparatus. After one hour the reaction was stopped, and the catalyst removed by filtration. The solvent was removed by rotary evaporation, and the residue was evaporated twice from acetonitril. Yield: 930 mg (88%). ¹H-NMR (D₂O): δ 3.11 ppm (t, 4H); 3.53-3.68 (m, 16H); 3.80 (m, 1H); 4.25 (s, 2H). ¹³C-NMR (D₂O): δ 38.18 ppm.; 65.43; 66.09; 68.55: 69.13; 69.23; 77.18; 173.42.

### Example 27

### 2-(1,3-Bis[2-(2-{9-fluorenylmethyloxycarbonylamino}ethoxy)ethoxy]propan-2-yloxy)acetic acid

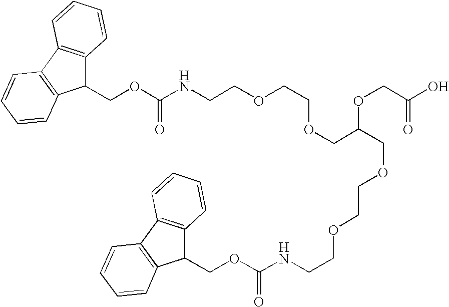

2-(1,3-bis[2-(2-aminoethoxy)ethoxy]propan-2-yloxy)acetic acid (9.35 g; 28.8 mmol) was added DIPEA (10 ml; 57 mmol). The reaction mixture was cooled on an ice bath, and chlorotrimethylsilane (15 ml; 118 mmol) dissolved in DCM (50 ml) was added dropwise, followed by DIPEA (11 ml; 62.7 mmol). To the almost clear solution was added dropwise a solution of Fmoc-Cl (15.0 g; 57 mmol) in DCM (50 ml). The reaction mixture was stirred overnight, then diluted with DCM (500 ml) and added to 0.01 N aqueous solution (500 ml). The organic layer was separated; washed with water (3×200 ml) and dried over anhydrous sodium sulfate. Solvent was removed by rotary evaporation. The crude product was purified by flash chromatography on silica using ethylacetate-heptane (1:1) as eluent. Pure fractions were collected and taken to dryness to give 9.20 g (42%) of title material.

¹H-NMR (D₂O): δ 3.34 ppm (t, 4H); 3.45-3.65 (m, 16H); 3.69 (bs, 1H); 4.20 (t, 2H); 4.26 (s, 2H); 4.38 (d, 4H); 5.60 (t, 2H); 7.30 (t, 4H); 3.35 (t, 4H); 7.58 (d, 4H); 7.72 (d, 4H). ¹³C-NMR (D₂O; selected peaks): δ 21.20 ppm.; 30.75; 34.64; 67.66; 68.90; 70.38; 70.51; 80.02; 120.37; 125.54; 127.48; 128.09; 128.67; 136.27; 141.69; 173.63; 176.80.

### Example 28

### 2-[2-(2-azidoethoxy)ethoxy]ethanol

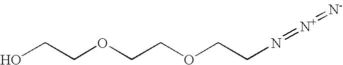

A slurry of 2-(2-(−2-chloroethoxy)ethoxy)ethanol (25.0 g, 148 mmol) and sodiumazide (14.5 g, 222 mmol) in dimethylformamide (250 ml) was standing at 100° C. night over. The reaction mixture was cooled on an ice bath, filtered and the organic solvent was evaporated in vacuo. The residue was dissolved in dichloromethane (200 ml), washed with water (75 ml), the water-phase was extracted with additional dichloromethane (75 ml) and the combined organic phases were dried with magnesium sulphate (MgSO₄), filtered and evaporated in vacuo giving an oil which was used without further purification. Yield: 30.0 g (100%). ¹³C-NMR (CDCl₃): δ 72.53; 70.66-70.05; 61.74; 50.65

### Example 29

### (2-[2-(2-Azidoethoxy)ethoxy]ethoxy)acetic acid

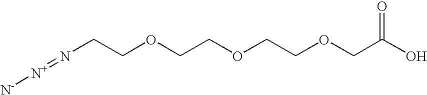

The above 2-[2-(2-azidoethoxy)ethoxy]ethanol (26 g, 148 mmol) was dissolved in tetrahydrofurane (100 ml) and under an nitrogen atmosphere slowly added to an ice cooled slurry of sodium hydride (24 g, 593 mmol, 60% in oil)) (which in advance had been washed with heptane (2×100 ml)) in tetrahydrofurane (250 ml). The reaction mixture was standing for 40 min. then cooled on a ice bath followed by slowly addition of bromoacetic acid (31 g, 223 mmol) dissolved in tetrahydrofurane (150 ml) and then standing about 3 hours at RT. The organic solvent was evaporated in vacuo. The residue was suspended in dichloromethane (400 ml). Water (100 ml) was slowly added, whereafter the mixture was standing for 30 min. under mechanical stirring. The water phase was separated, acidified with hydrochloride (4N) and extracted with dichloromethane (2×75 ml). All the combined organic phases were evaporated in vacuo giving a yellow oil. To the oil was slowly added a solution of piperidine (37 ml, 371 mmol) in dichloromethane (250 ml), the mixture was standing under mechanical stirring for 1 hour. The clear solution was diluted with dichloromethane (100 ml) and washed with hydrochloride (4N, 2×100 ml). The water phase was extracted with additional dichloromethane (2×75 ml) and the combined organic phases were evaporated in vacuo, giving an yellow oil which was used without further purification. Yield: 27.0 g (66%). ¹³C-NMR (CDCl₃): δ 173.30; 71.36; 70.66-70.05; 68.65; 50.65

### Example 30

### (S)-2,6-Bis-(2-{2-[2-(2-azidoethoxy)ethoxy]ethoxy}acetylamino)hexanoic acid methyl ester

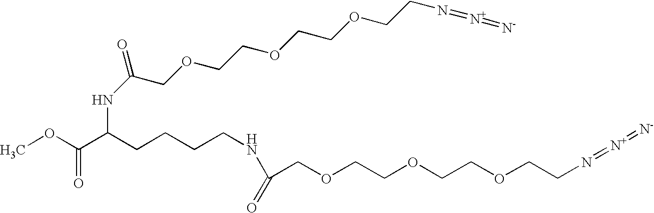

The above (2-[2-(2-azidoethoxy)ethoxy]ethoxy)acetic acid (13 g, 46.9 mol) was dissolved in dichloromethane (100 ml). N-Hydroxysuccinimide (6.5 g, 56.3 mmol) and 1-ethyl-3-(3-dimethylaminopropylcarbodiimide hydrochloride (10.8 g, 56.3 mmol) was added and the reaction mixture was standing for 1 hour. Diisopropylethylamine (39 ml, 234 mmol) and L-lysine methyl ester dihydrochloride (6.0 g, 25.8 mmol) were added and the reaction mixture was standing for 16 hours. The reaction mixture was diluted with dichloromethane (300 ml), extracted with water (100 ml), hydrochloride (2N, 2×100 ml), water (100 ml), 50% saturated sodiumhydrogencarbonate (100 ml) and water (2×100 ml). The organic phase was dried with Magnesium sulphate, filtered and evaporated in vacuo, giving an oil, which was used without further purification. Yield: 11 g (73%). LCMS: m/z=591. ¹³C-NMR (CDCl₃): (selected) δ 172.48; 169.87; 169.84; 71.093-70.02; 53.51; 52.34; 51.35; 50.64; 38.48; 36.48; 31.99; 31.40; 29.13; 22.82

### Example 31

### (S)-2,6-Bis-(2-{2-[2-(2-t-butyloxycarbonylaminoethoxy)ethoxy]ethoxy}acetylamino)hexanoic acid methyl ester

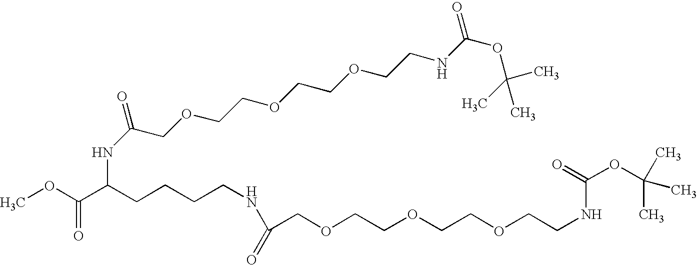

To a solution of the above (S)-2,6-bis-(2-{2-[2-(2-azidoethoxy)ethoxy]ethoxy}acetylamino)hexanoic acid methyl ester (1.0 g, 1.7 mmol) in ethylacetate (15 ml) was added di-tert-butyl dicarbonat (0.9 g, 4.24 mmol) and 10% Pd/C (0.35 g). Hydrogen was then constantly bubbled through the solution for 3 hours. The reaction mixture was filtered and the organic solvent was removed in vacuo. The residue was purified by flash chromatography using ethylacetate/methanol 9:1 as the eluent. Frations containing product were pooled and the organic solvent was removed in vacuo giving an oil. Yield: 0.60 g (50%). LC-MS: m/z=739 (M+1).

### Example 32

### (S)-2,6-Bis-(2-{2-[2-(2-aminoethoxy)ethoxy]ethoxy}acetylamino)hexanoic acid methyl ester

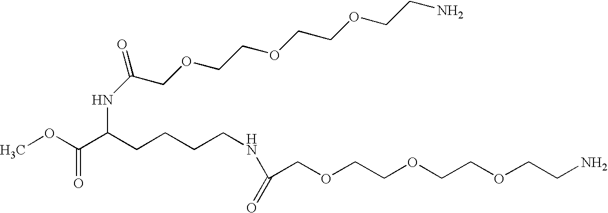

The above (S)-2,6-bis-(2-{2-[2-(2-t-butyloxycarbonylaminoethoxy)ethoxy]ethoxy}acetylamino) hexanoic acid methyl ester (0.6 g, 0.81 mmol) was dissolved in dichloromethane (5 ml). Trifluoroacetic acid (5 ml) was added and the reaction mixture was standing about 1 hour. The reaction mixture was evaporated, in vacuo, giving an oil, which was used without further purification. Yield: 0.437 g (100%). LC-MS m/z=539 (M+1)

### Example 33

### (S)-2,6-Bis-(2-{2-[2-(2-azidoethoxy)ethoxy]ethoxy}acetylamino)hexanoic acid

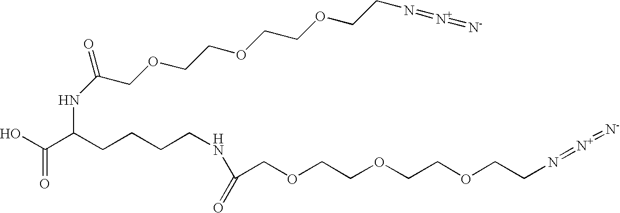

To a solution of (S)-2,6-bis-(2-{2-[2-(2-azidoethoxy)ethoxy]ethoxy}acetylamino) hexanoic acid methyl ester (2.0 g, 3.47 mmol) in methanol (10 ml) was added sodiumhydroxide (4N, 1.8 ml, 6.94 mmol) and the reaction mixture was standing for 2 hours. The organic solvent was evaporated in vacuo, and the residue was dissolved in water (45 ml) and acidified with hydrogenchloride (4N). The mixture was extracted with dichloromethane (150 ml) which was washed with saturated aqueous sodiumchloride (2×25 ml). The organic phase was dried over magnesium sulphate, filtered and evaporated, in vacuo, giving an oil. LC-MS m/z=577 (M+1).

### Example 34

### N-(tert-Butyloxycarbonylaminoxybutyl)phthalimide

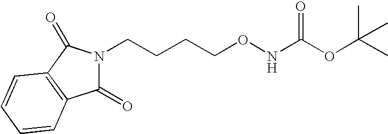

To a stirred mixture of N-(4-bromobutyl)phthalimide (18.9 g, 67.0 mmol), MeCN (14 ml), and N-Boc-hydroxylamine (12.7 g, 95.4 mmol) was added DBU (15.0 ml, 101 mmol) in portions. The resulting mixture was stirred at 50° C. for 24 h. Water (300 ml) and 12 M HCl (10 ml) were added, and the product was extracted three times with AcOEt. The combined extracts were washed with brine, dried (MgSO₄), and concentrated under reduced pressure. The resulting oil (28 g) was purified by chromatography (140 g SiO₂, gradient elution with heptane/AcOEt). 17.9 g (80%) of the title compound was obtained as an oil. ¹H NMR (DMSO-d₆) δ 1.36 (s, 9H), 1.50 (m, 2H), 1.67 (m, 2H), 3.58 (t, J=7 Hz, 2H), 3.68 (t, J=7 Hz, 2H), 7.85 (m, 4H), 9.90 (s, 1H).

### Example 35

### 4-(tert-Butyloxycarbonylaminoxy)butylamine

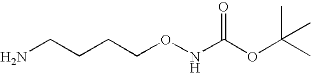

To a solution of N-(tert-butyloxycarbonylaminoxybutyl)phthalimide (8.35 g, 25.0 mmol) in EtOH (10 ml) was added hydrazine hydrate (20 ml), and the mixture was stirred at 80° C. for 38 h. The mixture was concentrated and the residue coevaporated with EtOH and PhMe. To the residue was added EtOH (50 ml), and the precipitated phthalhydrazide was filtered off and washed with EtOH (50 ml). Concentration of the combined filtrates yielded 5.08 g of an oil. This oil was mixed with a solution of K₂CO₃ (10 g) in water (20 ml), and the product was extracted with CH₂Cl₂. Drying (MgSO₄) and concentration yielded 2.28 g (45%) of the title compound as an oil, which was used without further purification. ¹H NMR (DMSO-d₆) δ 1.38 (m, 2H), 1.39 (s, 9H), 1.51 (m, 2H), 2.51 (t, J=7 Hz, 2H), 3.66 (t, J=7 Hz, 2H).

### Example 36

### 2-(2-Trityloxyethoxy)ethanol

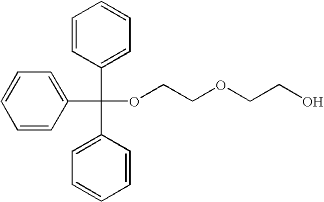

Tritylchloride (10 g, 35.8 mmol) was dissolved in dry pyridine, diethyleneglycol (3.43 mL, 35.8 mmol) was added and the mixture was stirred under nitrogen overnight. The solvent was removed in vacuo. The residue was dissolved in dichloromethane (100 mL) and washed with water. The organic phase was dried over Na₂SO₄ and solvent was removed in vacuo. The crude product was purified by recrystallization from heptane/toluene (3:2) to yield the title compound. ¹H NMR (CDCl₃): δ 7.46 (m, 6H), 7.28, (m, 9H), 3.75 (t, 2H), 3.68 (t, 2H), 3.62 (t, 2H), 3.28 (t, 2H). LC-MS: m/z=371 (M+Na); Rₜ=2.13 min.

### Example 37

### 2-[2-(2-Trityloxyethoxy)ethoxymethyl]oxirane

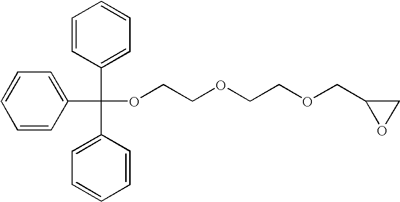

2-(2-Trityloxyethoxy)ethanol (6.65 g, 19 mmol) was dissolved in dry THF (100 mL). 60% NaH-oil suspension (0.764 mg, 19 mmol) was added slowly. The suspension was stirred for 15 min. Epibromohydrin (1.58 mL, 19 mmol) was added and the mixture was stirred under nitrogen at room temperature overnight. The reaction was quenched with ice, separated between diethyl ether (300 mL) and water (300 mL). The water phase was extracted with dichloromethane. The organic phases were collected, dried (Na₂SO₄) and solvent removed in vacou to afford an oil which was purified on silical gel column eluted with DCM/MeOH/Et₃N (98:1:1) to yield the title compound. ¹H NMR (CDCl₃): δ7.45 (m, 6H), 7.25, (m, 9H), 3.82 (dd, 1H), 3.68 (m, 6H), 3.45 (dd, 1H), 3.25 (t, 2H), 3.15 (m, 1H), 2.78 (t, 1H), 2.59 (m, 1H). LC-MS: m/z=427 (M+Na); Rₜ=2.44 min.

### Example 38

### 1,3-Bis[2-(2-trityloxyethoxy)ethoxy]propan-2-ol

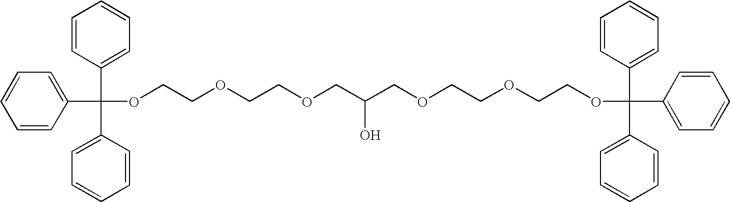

2-(2-Trityloxyethoxy)ethanol (1.14 g, 3.28 mmol) was dissolved in dry DMF (5 mL). 60% NaH-oil suspension (144 mg, 3.61 mmol) was added slowly and the mixture was stirred under nitrogen at room temperature for 30 min. The mixture was heated to 40° C. 2-[2-(2-Trityloxyethoxy)ethoxymethyl]oxirane (1.4 g, 3.28 mmol) was dissolved in dry DMF (5 mL) and added drop wise to the solution under nitrogen while stirring was maintained. After ended addition the mixture was stirred under nitrogen at 40° C. overnight. The heating was removed and after cooling to room temperature the reaction was quenched with ice and poured into saturated aqueous NaHCO₃ (100 mL). The mixture was extracted with diethyl ether (3×75 mL). The organic phases were collected, dried (Na₂SO₄), and solvent removed in vacuo to afford an oil which was purified on silical gel column eluted with EtOAc/Heptane/Et₃N (49:50:1) to yield the title compound.

¹H NMR (CDCl₃): δ7.45 (m, 12H), 7.25, (m, 18H), 3.95 (m, 1H), 3.78-3.45 (m, 16H), 3.22 (t, 4H), LC-MS: m/z=775 (M+Na); Rₜ=2.94 min.

### Example 39

### 1,3-Bis[2-(2-trityloxyethoxy)ethoxy]propan-2-yloxy β-cyanoethyl N,N-diisopropylphosphoramidite

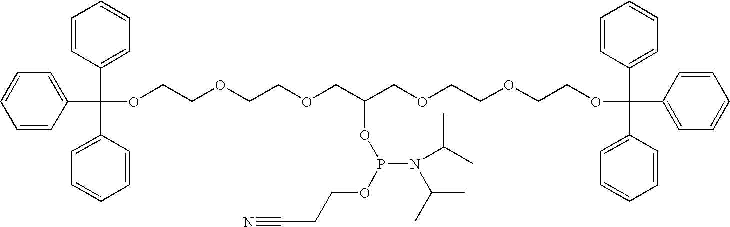

1,3-Bis[2-(2-trityloxyethoxy)ethoxy]propan-2-ol (0.95 g, 1.26 mmol) was evaporated twice from dry pyridine and once from dry acetonitrile. The residue was dissolved in dry THF (15 mL), while stirring under nitrogen. Diisopropylethylamine (1.2 mL, 6.95 mmol) was added. The mixture was coold to 0° C. with an icebath 2-cyanoethyl diisopropylchlorophosphoramidite (0.39 mL, 1.77 mmol) was added under nitrogen. The mixture was stirred for 10 minutes at 0° C. followed by 30 minutes at room temperature. Aqueous NaHCO₃ (50 mL) was added and the mixture extracted with DCM/Et₃N (98:2) (3×30 mL). The organic phases were collected, dried (Na₂SO₄), and the solvent removed in vacuo to afford an oil which was purified on silical gel column eluted with EtOAc/Heptane/Et₃N (35:60:5) to yield the 703 mg of title compound. ³¹P-NMR (CDCl₃): δ149.6 ppm.

### Example 40

### 2-(1,3-Bis[2-(2-hydroxyethoxy)ethoxy]propan-2-yloxy) acetic acid tert-butyl ester

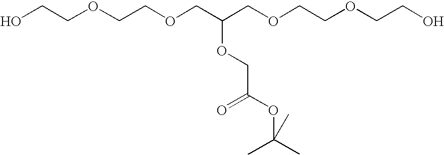

1,3-Bis[2-(2-trityloxyethoxy)ethoxy]propan-2-ol (0.3 g, 0.40 mmol) was evaporated once from dry pyridine and once from dry acetonitrile. The residual was dissolved in dry DMF (2 mL), under nitrogen, 60% NaH-oil suspension (24 mg, 0.6 mmol) was added. The mixture was stirred at room temperature for 15 minutes. tert-Butylbromoacetate (0.07 mL, 0.48 mmol) was added and the mixture was stirred for an additional 60 minutes. The reaction was quenched with ice, then partitioned between diethyl ether (100 mL) and water (100 mL). The organic phase was collected, dried (Na₂SO₄), and solvent removed in vacuo to afford an oil which was eluted on silical gel column with EtOAc/Heptane/Et₃N (49:50:1). Fraction containing main product was collected. The solvent was removed in vacuo and the residue was dissolved in 80% aqueous acetic acid (5 mL) and stirred at room temperature overnight. Solvent was removed in vacuo and the crude material dissolved in diethyl ether (25 mL), and washed with water (2×5 mL). The water phases were collected and the water removed on rotorvap to yield 63 mg of the title compound. ¹H NMR (CDCl₃): δ4.19 (s, 2H), 3.78-3.55 (m, 21H), 1.49 (s, 9H).

### Example 41

### N,N-Bis(2-(2-phthalimidoethoxy)ethyl)-O-tert-butylcarbamate

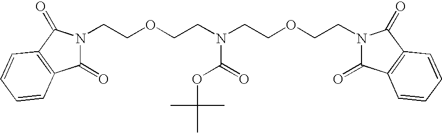

N,N-Bis(2-hydroxyethyl)-O-tert-butylcarbamate is dissolved in a polar, non-protic solvent such as THF or DMF. Sodium hydride (60% suspension in mineral oil) is added slowly to the solution. The mixture is stirred for 3 hours. N-(2-Bromoethyl)phthalimide is added. The mixture is stirred until the reaction is complete. The reaction is quenched by slow addition of methanol. Ethylacetate is added. The solution is washed with aqueous sodium hydrogencarbonate. The organic phase is dried, filtered, and subsequently concentrated under vacuum as much as possible. The crude compound is purified by standard column chromatography.

### Example 42

### N,N-Bis(2-(2-aminoethoxy)ethyl)-O-tert-butylcarbamate

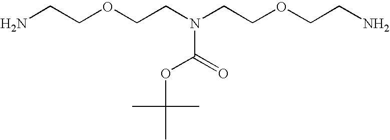

N,N-Bis(2-(2-phthalimidoethoxy)ethyl)-O-tert-butylcarbamate is dissolved in a polar solvent such as ethanol. Hydrazine (or another agent known to remove the phthaloyl protecting group) is added. The mixture is stirred at room temperature (or if necessary elevated temperature) until the reaction is complete. The mixture is concentrated under vacuum as much as possible. The crude compound is purified by standard column chromatography or if possible by vacuum destillation.

### Example 43

### N,N-Bis(2-(2-benzyloxycarbonylaminoethoxy)ethyl)-O-tert-butylcarbamate

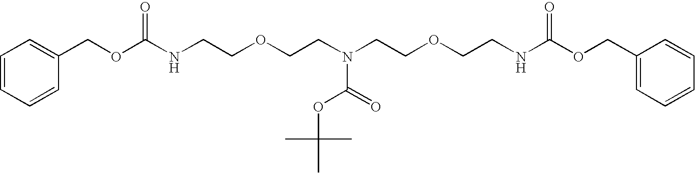

N,N-Bis(2-(2-aminoethoxy)ethyl)-O-tert-butylcarbamate is dissolved in a mixture of aqueous sodium hydroxide and THF or in a mixture of aqueous sodium hydroxide and acetonitrile. Benzyloxychloroformate is added. The mixture is stirred at room temperature until the reaction is complete. If necessary, the volume is reduced in vacuo. Ethyl acetate is added. The organic phase is washed with brine. The organic phase is dried, filtered, and subsequently concentrated in vacuo as much as possible. The crude compound is purified by standard column chromatography.

### Example 44

### Bis(2-(2-phthalimidoethoxy)ethyl)amine

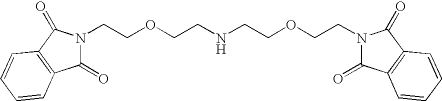

Bis(2-(2-phthalimidoethoxy)ethyl)-tert-butylcarbamate is dissolved in trifluoroacetic acid. The mixture is stirred at room temperature until the reaction is complete. The mixture is concentrated in vacuo as much as possible. The crude compound is purified by standard column chromatography.

### Example 45

### 11-Oxo-17-phthalimido-12-(2-(2-phthalimidoethoxy)ethyl)-3,6,9,15-tetraoxa-12-azaheptadecanoic acid

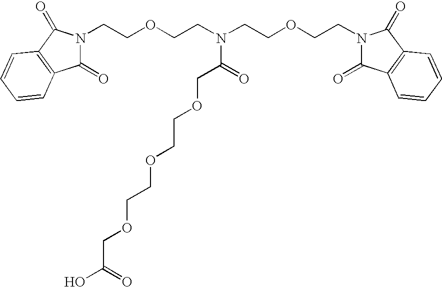

3,6,9-Trioxaundecanoic acid is dissolved in dichloromethane. A carbodiimide (e.g., N,N-dicyclohexylcarbodiimide or N,N-diisopropylcarbodiimide) is added. The solution is stirred over night at room temperature. The mixture is filtered. The filtrate can be concentrated in vacuo if necessary. The acylation of amines with the formed intramolecular anhydride is known from literature (e.g., Cook, R. M.; Adams, J. H.; Hudson, D. Tetrahedron Lett., 1994, 35, 6777-6780 or Stora, T.; Dienes, Z.; Vogel, H.; Duschl, C. Langmuir 2000, 16, 5471-5478). The anhydride is mixed with a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 46

### 5-Oxo-11-phthalimido-6-(2-(2-phthalimidoethoxy)ethyl)-3,9-dioxa-6-azaundecanoic acid

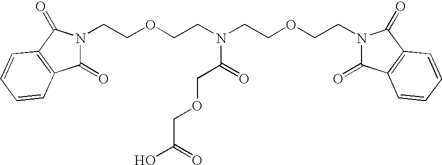

A solution of diglycolic anhydride in a non-protic solvent such as dichloromethane or N,N-dimethylformamide is added dropwise to a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 47

### 1,2,3-Benzotriazin-4(3H)-one-3-yl 2-[2-(2-methoxyethoxy)ethoxy]acetate

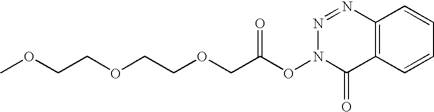

3-Hydroxy-1,2,3-benzotriazin-4(3H)-one (10.0 g; 61.3 mmol) and 2-[2-(2-methoxyethoxy)ethoxy]acetic acid (10.9 g; 61.3 mmol) was suspended in DCM (125 ml) and DIC (7,7 g; 61.3 mmol) was added. The mixture was stirred under a dry atmosphere at ambient temperature over night. A precipitate of diisopropyl urea was formed, which was filtered off. The organic solution was washed extensively with aqueous saturated sodium hydrogen carbonate solution, then dried (Na₂SO₄) and evaporated in vacuo, to give the title product as a clear yellow oil. Yield was 16.15 g (81%). ¹H-NMR (CDCl₃): δ 3.39 ppm (s, 3H); 3.58 (t, 2H); 3.68 (t, 2H); 3.76 (t, 2H); 3.89 (t, 2H); 4.70 (s, 2H); 7.87 (t, 1H); 8.03 (t, 1H); 8.23 (d, 1H); 8.37 (d, 1H). ¹³C-NMR (CDCl₃, selected peaks): δ 57.16 ppm; 64.96; 68.71; 68.79; 69.59; 69.99; 120.32; 123.87; 127.17; 130.96; 133.63; 142.40; 148.22; 164.97.

Oligomer Products

Solid Phase Oligomerization:

The reactions described below are all performed on polystyrene functionalized with the Wang linker. The reactions will in general also work on other types of solid supports, as well as with other types of functionalized linkers.

Solid Phase Azide Reduction:

The reaction is known (Schneider, S. E. et al. Tetrahedron, 1998, 54(50) 15063-15086) and can be performed by treating the support bound azide with excess of triphenyl phosphine in a mixture of THF and water for 12-24 hours at room temperature. Alternatively, trimethylphosphine in aqueous THF as described by Chan, T. Y. et al Tetrahedron Lett. 1997, 38(16), 2821-2824 can be used. Reduction of azides can also be performed on solid phase using sulfides such as dithiothreitol (Meldal, M. et al. Tetrahedron Lett. 1997, 38(14), 2531-2534) 1,2-dimercaptoethan and 1,3-dimercaptopropan (Meinjohanns, E. et al. J. Chem. Soc, Perkin Trans 1, 1997,6,871-884) or tin(II) salts such as tin(II)chloride (Kim, J. M. et al. Tetrahedron Lett, 1996, 37(30), 5305-5308).

Solid Phase Carbamate Formation:

The reaction is known and is usually performed by reacting an activated carbonate, or a halo formiate derivative with an amine, preferable in the presence of a base.

### Example 48

### 3-(1,3-Bis{2-[2-(1,3-Bis[2-(2-{2-[2-(2-methoxyethoxy)ethoxy]acetamino}ethoxy)ethoxy]propan-2-yloxy)acetylamino]ethoxy])ethoxy}propan-2-yloxy)acetylamino)propanoicacid

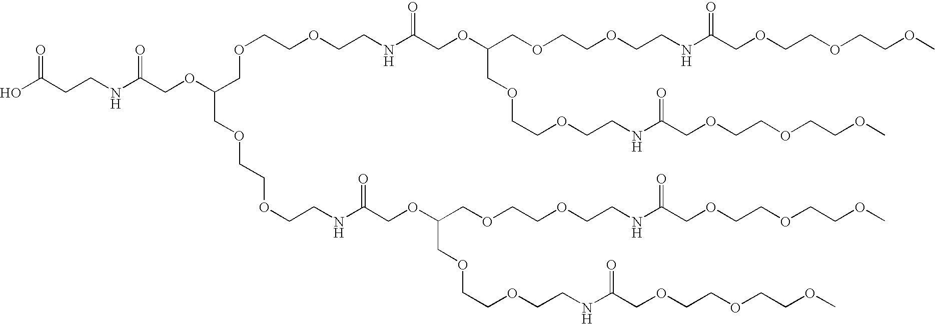

This example uses the 2-(1,3-Bis[azidoethoxyethyl]propan-2-yloxy)acetic acid monomer building block prepared in example 6 in the synthesis of a second generation amide based dendrimer capped with 2-[2-(2-methoxyethoxy)ethoxy]acetic acid. The coupling chemistry is based on standard solid phase peptide chemistry, and the protection methodology is based on a solid phase azide reduction step as described above.

Step 1: Fmoc-βala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 2-(1,3-bis[azidoethoxyethyl]propan-2-yloxy)acetic acid (527 mg; 1,4 mmol, 4×) and DhbtOH (225 mg; 1,4 mmol, 4×) were dissolved in DMF (5 ml) and DIC (216 ul, 1,4 mmol, 4×) was added. The mixture was left for 10 min (pre-activation) then added to the resin together with DIPEA (240 ul; 1,4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was redrawn and treated with a solution of benzoylchloride (0.5 M) and DIPEA (1 M) in DMF for 1 h. The resin was cleaved with 50% TFA/DCM and the dibenzoylated product analyzed with NMR and LC-MS. ¹H-NMR (CDCl₃): 3.50-3.75 (m, 20H); 3.85 (s, 1H); 4.25 (d, 2H); 6.95 (t, 1H); 7.40-7.50 (m, 6H); 7.75 (m, 4H). LC-MS (any-one): m/e=576 (M+1)⁺; Rₜ=2.63 min.

Step 5-7 was performed as step 2-4 using a double molar amount of reagents but same amount of solvent.

Step 8: capping with 2-[2-(2-methoxyethoxy)ethoxy]acetic acid: A solution of 2-[2-(2-methoxyethoxy)ethoxy]acetic acid (997 mg; 5.6 mmol, 16× with respect to resin loading) and DhbtOH (900 mg; 5.6 mmol, 16×) are dissolved in DMF (5 ml) and DIC (864 ul, 5.6 mmol, 16×) is added. The mixture is left for 10 min (pre-activation) then added to the resin together with DIPEA (960 ul; 5.6 mmol, 16×). The resin is shaken for 90 min, then drained and washed with DMF (3×) and DCM (3×).

Step 9: Cleavage from resin: The resin is treated with a 50% TFA-DCM solution at ambient temperature for 30 min. The solvent is collected and the resin is washed an additional time with 50% TFA-DCM. The combined filtrates are evaporated to dryness, and the residue purified by chromatography.

### Example 49

### 3-(1,3-Bis{2-[2-(1,3-bis[2-(2-{2-[2-(2-methoxyethoxy)ethoxy]acetamino}ethoxy)ethoxy]propan-2-yloxycarbonyl) amino]ethoxy])ethoxy}propan-2-yloxycarbonyl)amino)propanoicacid

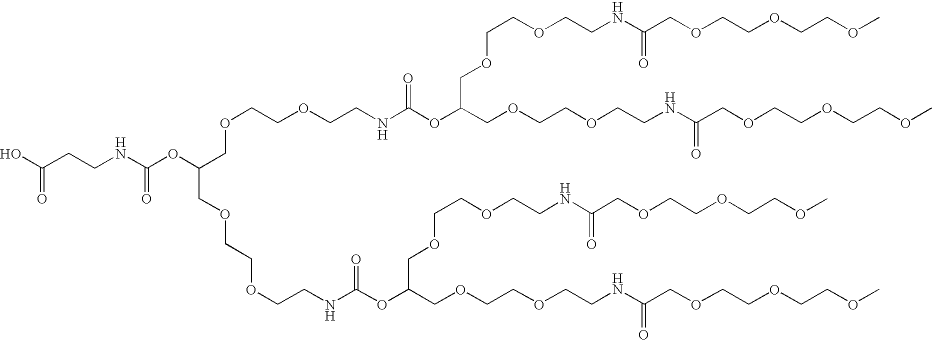

This example uses the 1,3-Bis[2-(2-azidoethoxy)ethoxy]porpan-2-yl-p-nitrophenylcarbonate monomer building block prepared in example 4 in the synthesis of a second generation carbamate based dendrimer capped with 2-[2-(2-methoxyethoxy)ethoxy]acetic acid. The coupling chemistry is based on standard solid phase carbamate chemistry, and the protection methodology is based on a solid phase azide reduction step as described above.

Step 1: Fmoc-βala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 1,3-Bis[azidoethoxyethyl]propan-2-yl-p-nitrophenylcarbamate (527 mg; 1,4 mmol, 4×). was added to the resin together with DIPEA (240 ul; 1,4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was redrawn and treated with a solution of benzoylchloride (0.5 M) and DIPEA (1 M) in DMF for 1 h. The resin was cleaved with 50% TFA/DCM and the dibenzoylated product analyzed with NMR and LC-MS. ¹H-NMR (CDCl₃): 3.50-3.75 (m, 20H); 3.85 (s, 1H); 4.25 (d, 2H); 6.95 (t, 1H); 7.40-7.50 (m, 6H); 7.75 (m, 4H). LC-MS (any-one): m/e=576 (M+1)⁺; Rₜ=2.63 min.

Step 5-7 was performed as step 2-4 using a double molar amount of reagents but same amount of solvent.

Step 8: capping with 2-[2-(2-methoxyethoxy)ethoxy]acetic acid: A solution of 2-[2-(2-methoxyethoxy)ethoxy]acetic acid (997 mg; 5.6 mmol, 16× with respect to resin loading) and DhbtOH (900 mg; 5.6 mmol, 16×) are dissolved in DMF (5 ml) and DIC (864 ul, 5.6 mmol, 16×) is added. The mixture is left for 10 min (pre-activation) then added to the resin together with DIPEA (960 ul; 5.6 mmol, 16×). The resin is shaken for 90 min, then drained and washed with DMF (3×) and DCM (3×).

Step 9: Cleavage from resin: The resin is treated with a 50% TFA-DCM solution at ambient temperature for 30 min. The solvent is collected and the resin is washed an additional time with 50% TFA-DCM. The combined filtrates are evaporated to dryness, and the residue purified by chromatography.

### Example 50

### 3-[2-(1,3-Bis[2-(2-{2-[2-(2-methoxyethoxy)ethoxy]acetylamino}ethoxy)ethoxy]propan-2-yloxy)acetylamino]propanoic acid

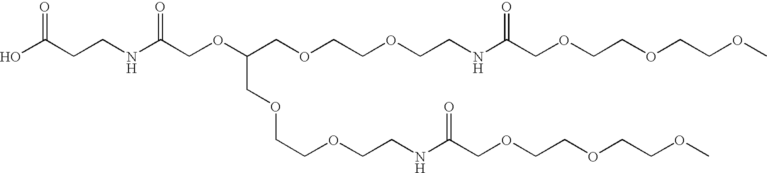

Step 1: Fmoc-β-alanine linked Wang resin (A22608, Nova Biochem, 3.00 g; with loading 0.83 mmol/g) was svelled in DCM for 20 min. then washed with DCM (2×20 ml) and NMP (2×20 ml). The resin was then treated twice with 20% piperidine in NMP (2×15 min). The resin was washed with NMP (3×20 ml) and DCM (3×20 ml).

Step 2: 2-(1,3-Bis[2-(2-azidoethoxy)ethoxy]propan-2-yloxy)acetic acid (3.70 g; 10 mmol) was dissolved in NMP (30 ml) and DhbtOH (1.60 g; 10 mmol) and DIC (1.55 ml; 10 mmol) was added. The mixture was stirred at ambient temperature for 30 min, then added to the resin obtained in step 1 together with DIPEA (1.71 ml; 10 mmol). The reaction mixture was shaken for 1.5 h, then drained and washed with NMP (5×20 ml) and DCM (3×20 ml).

Step 3: A solution of SnCl₂.2H₂O (11.2 g; 49.8 mmol) in NMP (15 ml) and DCM (15 ml) was then added. The reaction mixture was shaken for 1 h. The resin was drained and washed with NMP:MeOH (5×20 ml; 1:1). The resin was then dried in vacuo.

Step 4: A solution of 2-[2-(2-methoxyethyl)ethoxy]acetic acid (1.20 g; 6.64 mmol), DhbtOH (1.06 g; 6.60 mmol) and DIC (1.05 ml; 6.60 mmol) in NMP (10 ml) was mixed for 10 min, at room temperature, and then added to the 3-[2-(1,3-bis[2-(2aminoethoxy)ethoxy]propan-2-yloxy)acetylamino]propanoic acid tethered wang resin (1.0 g; 0.83 mmol/g) obtained in step 3. DIPEA (1.15 ml, 6.60 mmol) was added, and the reaction mixture was shaken for 2.5 h. Solvent was removed, and the resin was washed with NMP (5×20 ml) and DCM (10×20 ml).

Step 5: The resin product of step 4 was treated with TFA:DCM (10 ml, 1:1) for 1 hour. The resin was filtered and washed once with TFA:DCM (10 ml, 1:1). The combined filtrate and washing was then taken dryness, to give a yellow oil (711 mg). The oil was dissolved in 10% acetonitril-water (20 ml), and purified over two runs on a preparative HPLC apparatus using a C18 column, and a gradient of 15-40% acetonitril-water. Fractions were subsequently analysed by LC-MS. Fractions containing product were pooled and taken to dryness. Yield: 222 mg (37%). LC-MS: m/z=716 (m+1), Rₜ=1.97 min. ¹H-NMR (CDCl₃): δ 2.56 ppm (t, 2H); 3.36 (s, 6H); 3.46-3.66 (m, 39H); 4.03 (s, 4H); 4.16 (s, 2H); 7.55 (t, 2H); 8.05 (t, 1H). ¹³C-NMR (CDCl₃, selected peaks): δ 33.71 ppm; 34.90; 58.89; 68.94; 69.40; 69.98; 70.09; 70.33; 70.74; 70.91; 71.07; 71.74; 79.07; 171.62; 171.97; 173.63.

### Example 51

### 3-(1,3-Bis{2-(2-[2-(1,3-bis[2-(2-{2-[2-(2-methoxyethoxy)ethoxy]acetamino}ethoxy)-ethoxy]propan-2-yloxy)acetylamino]ethoxy)ethoxy}propan-2-yloxy)acetylamino)propanoic acid

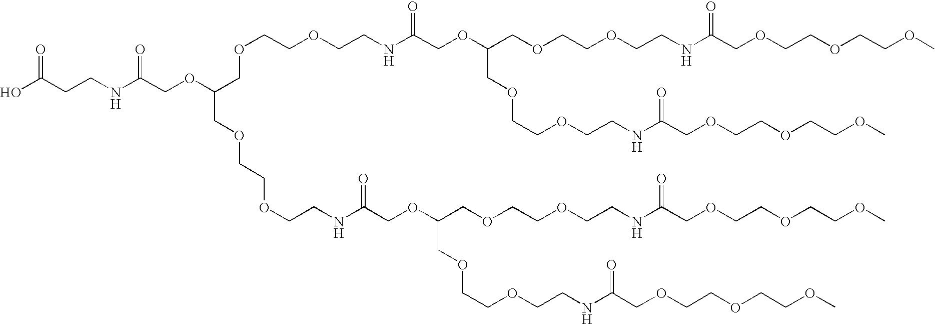

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 50 by repeating step 2-5, doubling the amount of reagents used. Yield: 460 mg (33%). MALDI-MS (α-cyanohydroxycinnapinic acid matrix): m/z=1670 (M+Na⁺). ¹H-NMR (CDCl₃): δ 2.57 ppm (t, 2H); 3.38 (s, 12H); 3.50-3.73 (m, 85H); 4.05 (s, 8H); 4.17 (s, 2H); 4.19 (s, 4H); 7.48 (m, 4H); 7.97 (m, 3H). ¹³C-NMR (CDCl₃, selected peaks): δ 38.81 ppm; 58.92; 69.46; 69.92; 70.05; 70.05; 70.13; 70.40; 70.73; 70.97; 71.11; 71.88; 76.74; 77.06; 77.38; 171.33; 172.02.

### Example 52

### 3-(1,3-Bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoic acid

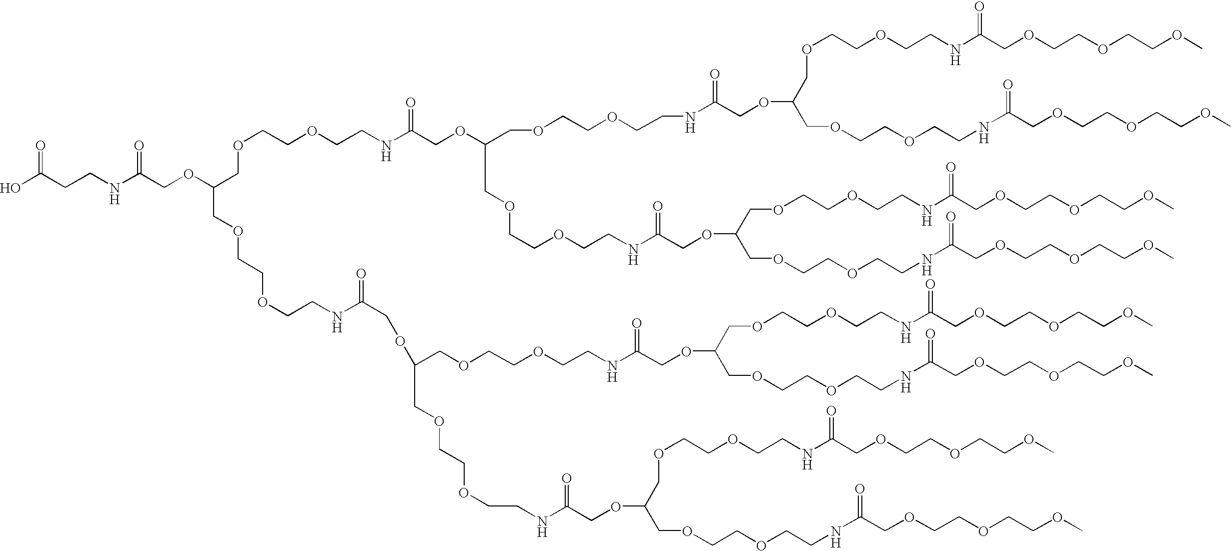

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 50 by repeating step 2-3 with 2× the amount of reagents used, then repeating step 2-5 with 4× the amount of reagent used. Yield: 84 mg (4%/). LC-MS: (m/2)+1=1758; (m/3)+1=1172; (m/4)+1=879; (m/5)+1=704. Rt=2.72 min. ¹H-NMR (CDCl₃): δ 2.51 ppm (t, 2H); 3.33 (s, 24H); 3.44-3.70 (m, 213H); 3.93 (s, 16H); 4.08 (s, 14H); 7.25 (m, 8H); 7.69 (m, 7H). ¹³C-NMR (CDCl₃₁ selected peaks): δ 38.94 ppm; 59.33; 69.78; 70.08; 70.37; 70.44; 70.56; 70.82; 71.10; 71.26; 71.51; 72.17; 79.24; 170.60; 171.22.

### Example 53

### N-Hydroxysuccinimidyl 3-[2-(1,3-bis[2-(2-{2-[2-(2-methoxyethoxy)ethoxy]acetylamino}ethoxy)ethoxy]propan-2-yloxy)acetylamino]propanoate

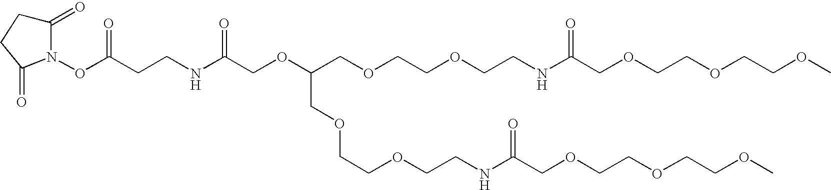

3-[2-(1,3-Bis[2-(2-{2-[2-(2-methoxyethoxy)ethoxy]acetylamino}ethoxy)ethoxy]propan-2-yloxy)acetylamino]propanoic acid (67 mg; 82 mmol) was dissolved in THF (5 ml). The reaction mixture was cooled on an icebath. DIPEA (20 ul; 120 mmol) and TSTU (34 mg; 120 mmol) was added. The mixture was stirred at ambient temperature overnight at which time, the reaction was complete according to LC-MS. LC-MS: m/z=813 (M+H)⁺; Rₜ=2.22 min.

### Example 54

### N-Hydroxysuccinimidyl 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanoate

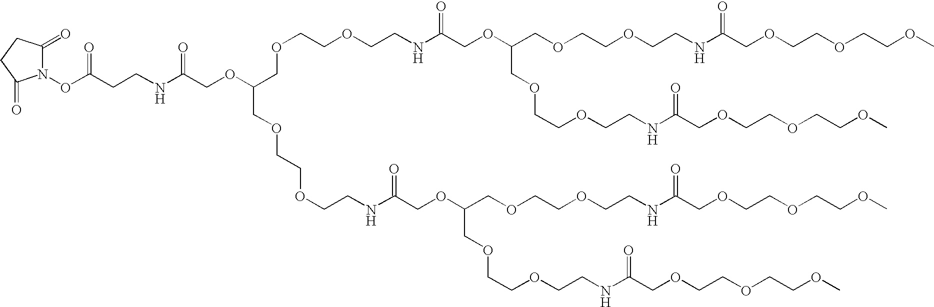

Prepared from 3-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)propanoic acid and TSTU as described in example 53. LC-MS: (m/2)+1=873, Rₜ=2.55 min.

### Example 55

### N-Hydroxysuccimidyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoate

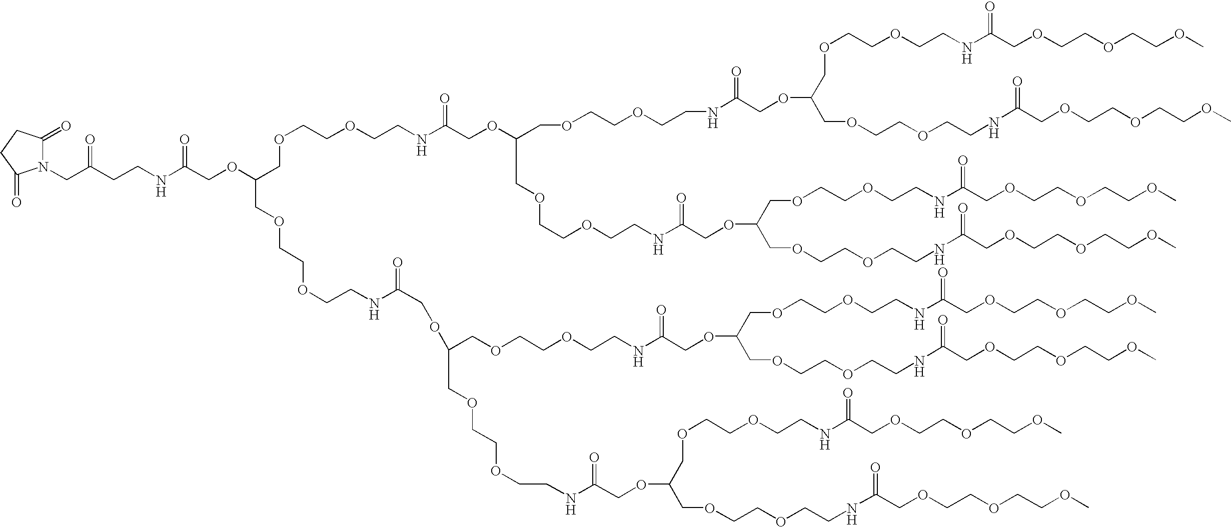

Prepared from N-hydroxysuccinimidyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoic acid and TSTU as described in example 53. LC-MS: (m/4)+1=903, Rₜ=2.69 min.

### Example 56

### N-(4-tert-Butoxycarbonylaminoxybutyl)3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

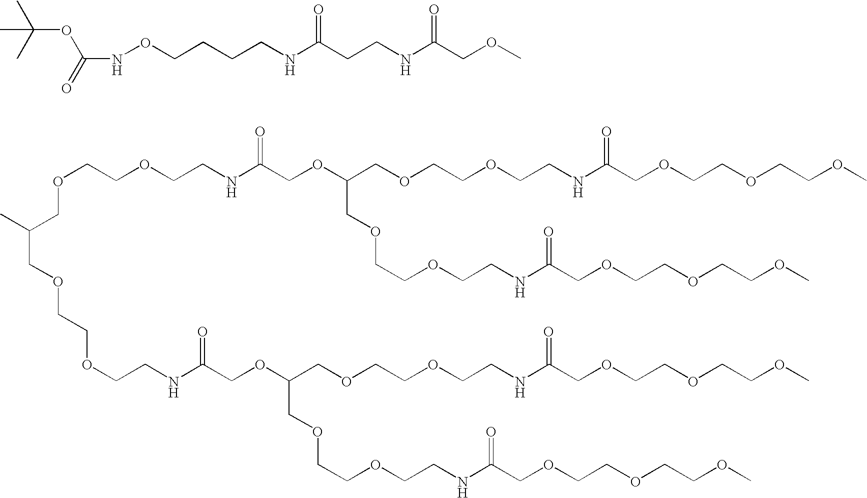

N-Hydroxysuccinimidyl 3-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)propanoate (105 mg; 0.06 mmol) was dissolved in DCM (2 ml). Then a solution of 4-(tert-butyloxycarbonylaminoxy)butylamine (49 mg; 0.24 mmol) was added followed by DIPEA (13 ul; 0.07 mmol). The mixture was stirred at ambinet temperature for one hour, then concentrated under reduced presure. The residual was dissolved in 20% acetonitril-water (4 ml), and purified on a preparative HPLC apparatus using a C18 column, and a step gradient of 0, 10, 20, 30, and 40% (10 ml elutions each) of acetonitril-water. Fractions containing pure product was concentrated and dried for 16 h in a vacuum oven to give a yellow oil. Yield: 57 mg (51%). LC-MS: (m/2)+1=918, Rt=2.75 min. ¹H-NMR (CDCl₃): δ 1.42 ppm (s, 9H); 2.40 (t, 2H); 3.21 (dd, 2H); 3.33 (s, 12H); 3.38-3.72 (m, 99H); 3.80 (m, 2H); 3.95 (s, 8H); 4.08 (s, 6H); 6.99 (m, 1H); 7.23 (m, 4H); 7.69 (m, 2H); 7.85 (m, 1H); 8.00 (m, 1H). ¹³C-NMR (CDCl₃, selected peaks): δ 28.27 ppm; 38.58; 58.97; 69.42; 69.72; 70.01; 70.08; 70.20; 70.41; 70.46; 70.73; 70.91; 71.16; 71.22; 71.81; 78.89; 81.33; 170.27; 170.89.

### Example 57

### N-(4-Aminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

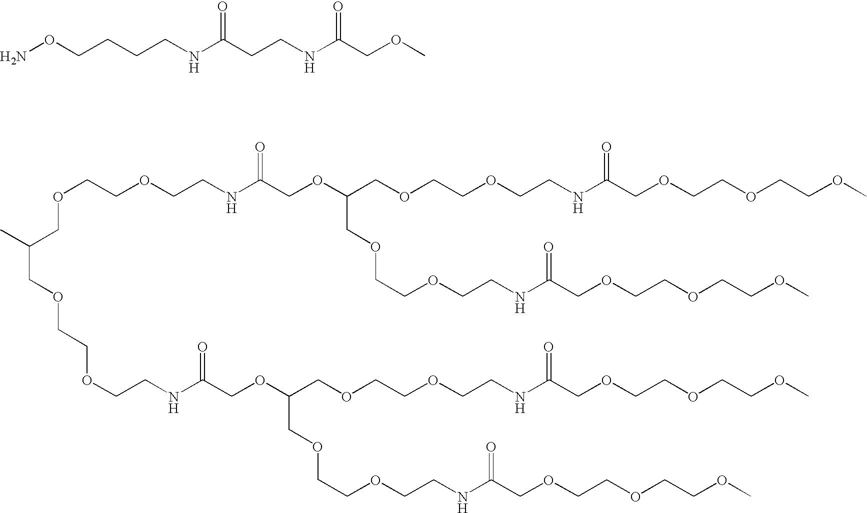

N-(4-tert-Butoxycarbonylaminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide (19 mg; 10 mmol) was dissolved in 50% TFA/DCM (10 ml), and the clear solution was stirred at ambient temperature for 30 min. The solvent was removed by rotaryevaporation, and the residue was stripped twice from DCM, to give a quantitative yield (19 mg) of the title product. LC-MS: (m/2)+1=868, (m/3)+1=579, Rt=2,35 min.

The following example illustrates solution phase conjugation to peptide or proteins.

### Example 58

### t-Butyl 2-(1,3-bis[2-(2-{2-[2-(2-methoxyethoxy)ethoxy]acetamino}ethoxy)-ethoxy]propan-2-yloxy)acetate

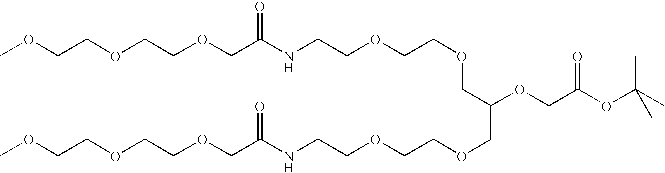

t-Butyl 2-(1,3-bis[2-(2-aminoethoxy)ethoxy]propan-2-yloxy)acetate (1.74 g; 4.5 mmol) and 1,2,3-benzotriazin-4(3H)-one-3-yl 2-[2-(2-methoxyethoxy)ethoxy]acetate (2.94 g; 9 mmol) was dissolved in DCM (100 ml). DIPEA (3.85 ml; 22.3 mmol) was added and the celar mixture was stirred for 90 min at room temperature. Solvent was removed in vacuo, and the residue was purified by chromatography on silica, using MeOH-DCM (1:16) as eluent. Pure fractions were pooled and taken to dryness to give the title material as a clear oil. Yield was 1.13 g (36%). ¹H-NMR (CDCl₃): δ 1.46 ppm (s, 9H); 3.38 (s, 6H); 3.49-3.69 (m, 37H); 4.01 (s, 4H); 4.18 (s, 2H); 7.20 (bs, 2H).

### Example 59

### 2-(1,3-Bis[2-(2-{2-[2-(2-methoxyethoxy)ethoxy]acetamino}ethoxy)ethoxy]propan-2-yloxy)acetic acid

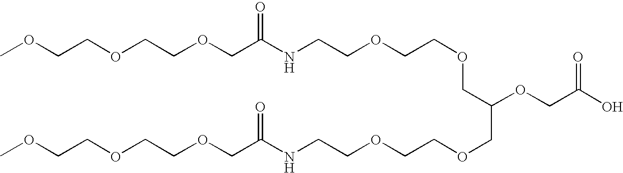

t-Butyl 2-(1,3-bis[2-(2-aminoethoxy)ethoxy]propan-2-yloxy)acetate (470 mg; 0.73 mmol) was dissolved in DCM-TFA (25 ml, 1:1) and the mixture was stirred for 30 min at ambient temperature. The solvent was removed, in vacuo, and the residue was stripped twice from DCM. LC-MS: (m+1)=645, Rt=2,26 min. ¹H-NMR (CDCl₃): δ 3.45 ppm (s, 6H);

3.54-3.72 (m, 37H); 4.15 (s, 4H); 4.36 (s, 2H).

### Example 60

### N-Hydroxysuccimidyl 2-(1,3-bis[2-(2-{2-[2-(2-methoxyethoxy)ethoxy]acetamino}ethoxy)ethoxy]propan-2-yloxy)acetate

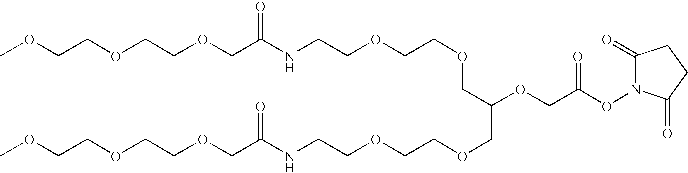

2-(1,3-Bis[2-(2-{2-[2-(2-methoxyethoxy)ethoxy]acetamino}ethoxy)ethoxy]propan-2-yloxy)acetic acid (115 mg; 0.18 mmol) was dissolved in THF (5 ml). The reaction mixture was placed on an ice bath. TSTU (65 mg, 0.21 mmol) and DIPEA (37 ul; 0.21 mmol) was added and the reaction mixture was stirred at 0° C. for 30 min, then at room temperature overnight. The reaction was then taken to dryness, to give 130 mg of the title material as an clear oil. LC-MS: (m+1)=743, (m/2)+1=372, Rt=2,27 min.

### Example 61

### tButyl 3-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetate

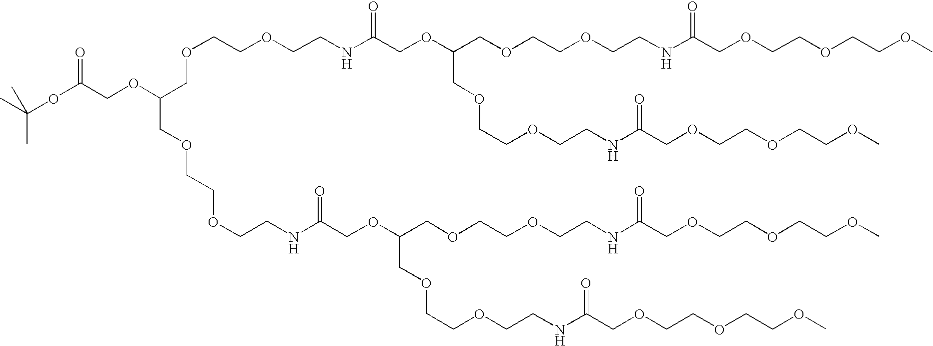

The material is prepared from two equivalents of N-hydroxysuccimidyl 2-(1,3-bis[2-(2-{2-[2-(2-methoxyethoxy)ethoxy]acetamino}ethoxy)ethoxy]propan-2-yloxy)acetate and one equivalent of t-Butyl 2-(1,3-bis[2-(2-aminoethoxy)ethoxy]propan-2-yloxy)acetate, using the protocol and purification method described in example 58. Subsequent removal of t-butyl group is done as described in example 59 and N-hydroxysuccimidyl ester formation is done as described in example 60.

### Example 62

### (S)-2,6-Bis-(2-[2-(2-[2-(2,6-bis-[2-(2-[2-(2-azidoethoxy)ethoxy]ethoxy)acetylamino]hexanoylamino)ethoxy]ethoxy)ethoxy]acetylamino)hexanoic acid methyl ester

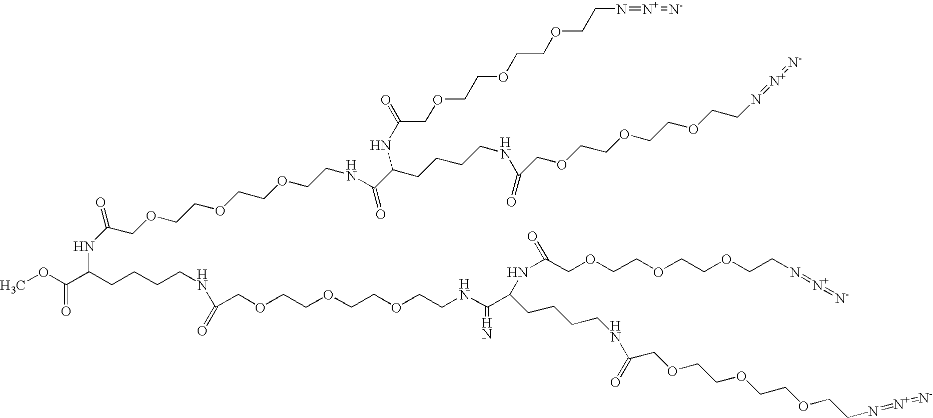

(S)-2,6-Bis-(2-{2-[2-(2-azidoethoxy)ethoxy]ethoxy}acetylamino)hexanoic acid (1.8 g, 3.10 mmol)) was dissolved in a mixture of dimethylformamide/dichloromethane 1:3 (10 ml), pH was adjusted to basic reaction using diisopropylethylamine, N-hydroxybenzotriazole and 1-ethyl-3-(3-dimethylaminopropylcarbodiimide hydrochloride were added and the reaction mixture was standing for 30 min. Then this reaction mixture was added to a solution of (S)-2,6-bis-(2-{2-[2-(2-aminoethoxy) ethoxy]ethoxy}acetylamino)hexanoic acid methyl ester (0.37 g, 0.70 mmol) in dichloromethane) and the reaction mixture was standing night over.

The reaction mixture was diluted with dichloromethane (150 ml), washed with water (2×40 ml), 50% saturated sodiumhydrogencarbonate (2×30 ml) and water (3×40 ml). The organic phase was dried over magnesium sulphate, filtered and evaporated in vacuo giving an oil. Yield: 1.6 g (89%). LC-MS: m/z=1656 (M+1) and m/z=828.8 (M/2)+1 and m/z=553(M/3)+1.

### Example 63

### (S)-2,6-Bis-(2-[2-(2-[2-((S)-2,6-bis-[2-(2-[2-(2-tert-butoxycarbonylaminoethoxy)ethoxy]ethoxy)acetylamino]hexanoylamino)ethoxy]ethoxy)ethoxy]acetylamino)hexanoic acid methyl ester

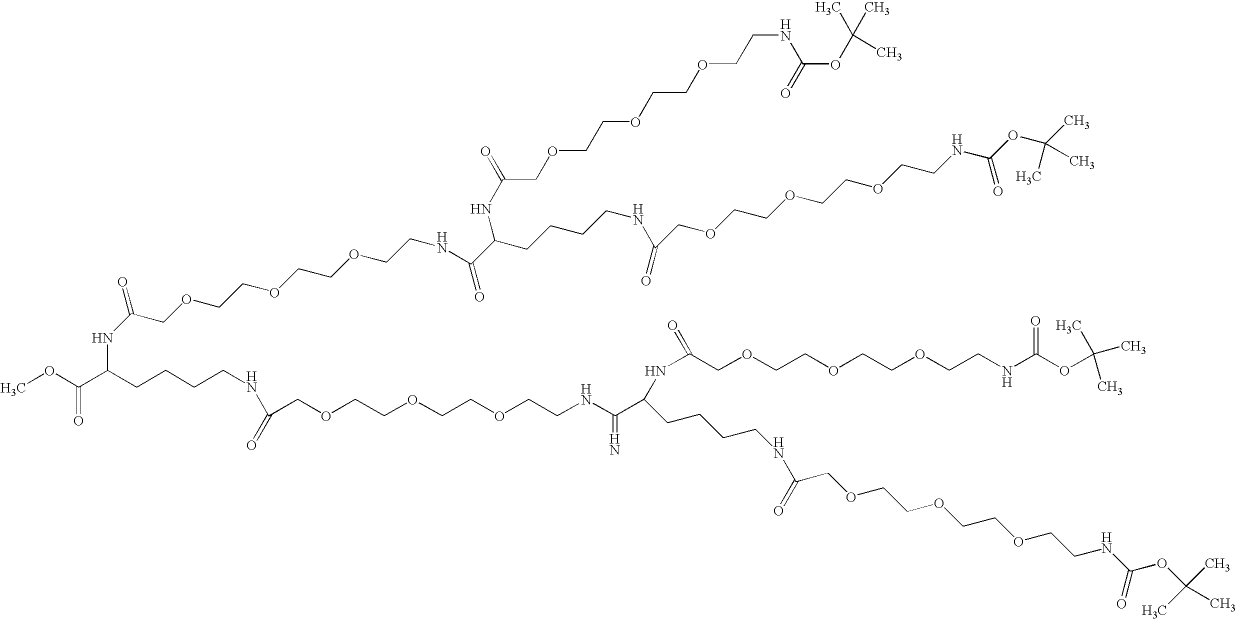

To a solution of the above (S)-2,6-Bis-(2-[2-(2-[2-((S)-2,6-bis-[2-(2-[2-(2-azidoethoxy)ethoxy]ethoxy)acetylamino]hexanoylamino)ethoxy]ethoxy)ethoxy]acetylamino)hexanoic acid methyl ester (1.6 g, 0.97 mmol) in ethylacetate (60 ml), was added di-tert-butyl dicarbonate (1.0 g, 4.8 mmol) and Pd/C (10%, 1.1 g). Hydrogen was constantly bubbled through the reaction mixture for 2 hours. The reaction mixture was filtered and the organic solvent was removed in vacuo giving an oil which was used without further purification. Yield: 1.8 g (98%). LC-MS: m/z=1953 (M+1) and m/z=977 (M/2)+1.

### Example 64

### (S)-2,6-Bis-(2-[2-(2-[2-((S)-2,6-bis-[2-(2-[2(2aminoethoxy)ethoxy]ethoxy)acetylamino]hexanoylamino)ethoxy]ethoxy)ethoxy]acetylamino)hexanoic acid methyl ester

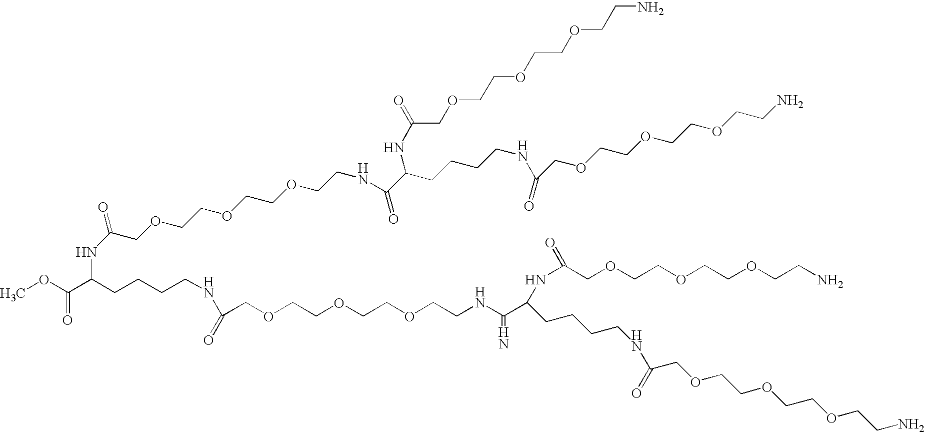

The above (S)-2,6-bis-(2-[2-(2-[2-((S)-2,6-bis-[2-(2-[2-(2-tert-butoxycarbonylaminoethoxy)ethoxy]ethoxy)acetylamino]hexanoylamino)ethoxy]ethoxy)ethoxy]-acetylamino)hexanoic acid methyl ester was dissolved in dichloromethane (20 ml) and trifluoroacetic acid (20 ml) was added. The reaction mixture was standing for 2 hours. The organic solvent was evaporated in vacuo, giving an oil.

Yield: 1.4 g (100%). LC-MS: m/z=1552 (M+1); 777.3 (M/2)+1; 518.5 (M/3)+1 and 389.1 (M/4)+1.

### Example 65

### (S)-2,6-Bis-(2-[2-(2-[2-((S)-2,6-bis-[2-(2-[2-(2-(2-(2-(2-methoxyethoxy)ethoxy) acetylamino)ethoxy)ethoxy]ethoxy)acetylamino]hexanoylamino)ethoxy]ethoxy) ethoxy]acetylamino)hexanoic acid methyl ester

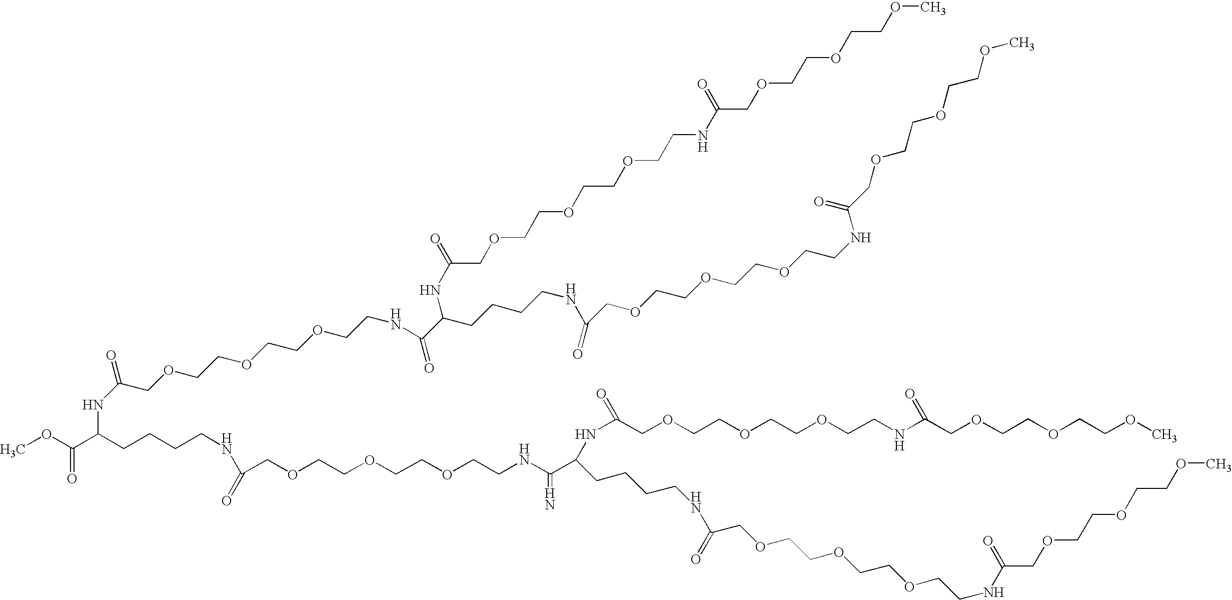

To a solution of 2-(2-(methoxyethoxy)ethoxy)acetic acid (1.3 g, 7.32 mmol) in a mixture of dichloromethane and dimethylformamide 3:1 (20 ml) was added N-hydroxysuccinimide (0.8 g, 7.32 mmol) and 1-ethyl-3-(3-dimethylaminopropylcarbodiimide hydrochloride (1.4 g, 7.32 mmol). The reaction mixture was standing for 1 hour, where after the mixture was added to a solution of (S)-2,6-bis-(2-[2-(2-[2-((S)-2,6-bis-[2-(2-[2(2aminoethoxy)ethoxy]ethoxy)acetylamino]hexanoylamino)ethoxy]ethoxy)ethoxy]acetylamino)hexanoic acid methyl ester (1.42 g, 0.92 mmol) and diisopropylethylamine (2.4 ml, 14.64 mmol) in dichloromethane (10 ml). The reaction mixture was standing night over. The reaction mixture was diluted with dichloromethane (100 ml) and extracted with water (3×25 ml). The combine water-phases were extracted with additional dichloromethane (2×75 ml). The combined organic phases were dried over magnesium sulphate filtered and evaporated in vacuo. The residue was purified by flash chromatography using 500 ml ethyl acetate, followed by 500 ml ethyl acetate/methanol 9:1 and finally methanol as the eluent. Fractions containing product were evaporated in vacuo giving an oil. Yield: 0.75 g (38%). LC-MS: m/z=1097 (M/2)+1; 732 (M/3)+1 and 549 (M/4)+1.

The (S)-2,6-Bis-(2-[2-(2-[2-((S)-2,6-bis-[2-(2-[2-(2-(2-(2-(2-methoxyethoxy)ethoxy) acetylamino)ethoxy)ethoxy]ethoxy)acetylamino]hexanoylamino)ethoxy]ethoxy) ethoxy]acetylamino)hexanoic acid methyl ester can be saponified to the free acid and attached to an amino group of a peptide or protein using via an activated ester. The activated ester may be produced and coupled to the amino group of the peptide or protein by standard coupling methods known in the art such as diisopropylethylamine and N-hydroxybenzotriazole or other activating conditions.

Further convertion according to FIG. 16 or analogue to example 53 and example 56 and 57 gives a dendritic material that can be used to prepare conjugates according to the invention.

### Example 66

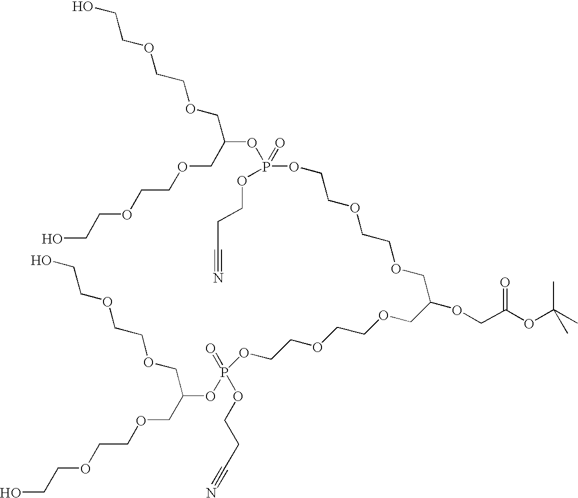

2-(1,3-Bis[2-(2-hydroxyethoxy)ethoxy]propan-2-oxy) acetic acid tert-butyl ester (63 mg, 0.16 mmol) was evaporated twice from dry acetonitrile. 1,3-Bis[2-(2-trityloxyethoxy)ethoxy]propan-2-oxy β-cyanoethyl N,N-diisopropylphosphoramidite (353 mg, 0.37 mmol) was evaporated twice from dry acetonotrile, dissolved on dry acetonitrile (2 mL) and added. A solution of tetrazole in dry acetonitrile (0.25 M, 2.64 mL) was added under nitrogen and the mixture was stirred at room temperature for 1 hour. 5.5 mL of an 12-solution (0.1 M in THF/Lutidine/H₂O 7:2:1) was added and the mixture was stirred an additional 1 hour. The reaction mixture was diluted with ethyl acetate (20 mL) and washed with 2% aqueous sodium sulfite until the iodine colour disappeared. The organic phase was dried (Na₂SO₄), and solvent removed in vacuo. The residue was dissolved in 80% aqueous acetic acid (5 mL) and stirred at room temperature overnight. Solvent was removed in vacuo and the crude material was added diethyl ether (25 mL) and water (10 mL). The water phase was collected and water removed in vacuo. Product was purified on reverse phase preparative HPLC C-18 colum, gradient 0-40% acetonitrile containing 0.1% TFA to give the tert-butyl-protected 2. generation dendrimer product.

HPLC-MS: m/z=1171 (M+Na); 1149 (M+), 1093 (lost of tert-butyl in the MS) Rₜ=2.76 min.

Deprotection of β-cyanoethyl groups and removal of tert-butyl ester group, is subsequently done using conventional base and acid treatments as known to the person skilled in the art.

Further convertion according to FIG. 16 or analogue to example 53 and example 56 and 57 gives a dendritic material that can be used to prepare conjugates according to the invention.

### Example 67

Asialo rFVIIa (10.2 mg, 0.2 mmol) in 13.5 ml TRIS buffer (10 mM Cacl2, 10 mM TRIS, 50 mM NaCl, 0.5% Tween 80, pH 7.4) was cooled on an icebath. A solution of N-(4-aminoxybutyl)3-(1,3-bis{2-(2-[2-(1,3-bis[2-(2-{2-[2-(2-methoxyethoxy)ethoxy]acetamino}ethoxy)ethoxy]-propan-2-yloxy)acetylamino]ethoxy)ethoxy}propan-2-yloxy)acetylamino)propanamide (17 mg; 10.0 mmol, 50×) in 2.5 ml TRIS buffer was added, followed by a solution of galactose oxidase (135 U) and catalase (7500 U) in 2.5 ml TRIS buffer. The reaction mixture was shaken gently for 48 h at 4° C. The slightly unclear solution was then filtered through a 0.45 um filter (Sartorius Minisart®). The buffer was then exchanged to MES (10 mM CaCl₂, 10 mM MES, 50 mM NaCl, pH 6.0) using a NAP-10 columns (Amersham). The mixture was then cooled on ice, and an aqueous solution of EDTA (3.5 ml, 100 mM, pH 8.0, equivalent to [Ca²⁺ ]) was added. pH was adjusted to 7.6 by addition of 1 M aqueous NaOH, and the sample (6.8 mS/cm) was loaded on a 5 ml HiTrap-Q HP ion-exchange column (Amersham-Biosciences), equilibrated with 10 mM Tris, 50 mM NaCl, pH 7.4. The column was eluted with 10 mM Tris, 50 mM NaCl, pH 7.4 (10 vol, flow: 1 ml/min). The elution buffer was then changed to 10 mM Tris, 50 mM NaCl, 25 mM CaCl₂, pH 7.4 (10 vol, flow: 1 ml/min). The eluates were monitored by UV, and each fraction containing protein was analyzed by SDS-PAGE gel electrophoresis. Pure samples of N-glycan modified rFVII were pooled and stored at −80° C.

1. A method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded thereto,
the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality; and
(b) contacting the glycoprotein product produced in step (a) with a reactant X capable of reacting with an aldehyde group; wherein reactant X comprises a protractor group to create a conjugate represented by the formula (glycoprotein)-(protractor group).

2. A method for producing a conjugate of a glycoprotein having increased in vivo plasma half-life compared to the non-conjugated glycoprotein, the conjugate comprising a glycoprotein having at least one terminal galactose or derivative thereof, and a protractor group covalently bonded to the thereto through a linking moiety;
the method comprising the steps of:
(a) contacting a glycoprotein having at least one terminal galactose or derivative thereof with galactose oxidase to create a glycoprotein comprising an oxidized terminal galactose or derivative thereof having a reactive aldehyde functionality;
(b) contacting the glycoprotein product produced in step (a) with a reactant X capable of reacting with an aldehyde group, wherein reactant X comprises a linking moiety further comprising a second reactive, optionally protected, group to create a conjugate of the glycoprotein and the linking moiety; and
(c) contacting the product of step (b) with a protractor group capable of reacting with the second reactive group of the linking moiety to create a conjugate represented by the formula (glycoprotein)-(linking moiety)-(protractor group).

3. A method according to claim 1, further comprising step (a1) and step (a2) to be carried out before said step (a):
(a1) contacting a glycoprotein with one or more of sialidases, galacosidases, N-acetylhexosaminidases, fucosidases, mannosidases, endo H and endo F3 to create a glycoprotein where part of the glycan structure is removed,
(a2) contacting the product of step (a1) with a galactosyltransferase and a galactose substrate to create a glycoprotein with at least one terminal residue of galactose or a derivative thereof.

4. A method according to claim 1, further comprising a first step (a3) to be carried out before said step (a):
(a3) contacting a glycoprotein having at least one terminal sialic acid residue with sialidase or another reagent capable of removing sialic acid from the glycans to create an asialo glycoprotein comprising at least one terminal galactose or derivative thereof.

5. A method according to any one of claim 1, wherein the reactant X has the formula nuc-R, wherein nuc is a functional group which can react with an aldehyde to form a covalent bond, and R is a protractor group.

6. A method according to claim 2, wherein the reactant X has the formula nuc-L1-R, wherein nuc is a functional group which can react with an aldehyde to form a covalent bond, R is a protractor group, and L1 is a linking moiety.

7. A method according to claim 5, wherein the reactant X is selected from the group consisting of: H₂N—R, HR1N—R, H₂N—O—R, HR1N—O—R, H₂N—NH—CO—R, H₂N—CHR1—CHR—SH, H₂N—CHR—CHR1-SH, H₂N—NH—SO₂—R, and Z′-CH₂-Z″-R; wherein R is a protractor group; R1 is H or a second protractor group; Z′ and Z″ represent electron withdrawing groups; and one or both of the Z groups can be connected to the R group.

8. A method according to claim 6, wherein the reactant X is selected from the group of H₂N-L1—R, HR1; N-L1-R, H₂N—O-L1-R, HR1N—O-L1-R, H₂N—NH—CO-L1-R, H₂N—CHR1-CH(L1-R)—SH, H₂N—CH(L1-R)—CHR1-SH, H₂N—NH—SO₂-L1-R, Z′-CH₂-Z″-L1-R; wherein L1 is a linking moiety, R is a protractor group, R1 is H or a second protractor group; Z′ and Z″ represent electron withdrawing groups; and wherein one or both of the Z groups can be connected to the R group.

9. A method according to claim 2, wherein the reactant X has the formula nuc-L1-Rₙ, wherein nuc is a functional group which can react with an aldehyde to form a covalent bond; R is a protractor group; L1 is a polyfunctional linking moiety connecting one or more protractor groups (R) to the reactive functionality (nuc); and wherein L1 may or may not contribute to the protraction; and n represents an integer, n=1-25, such as 1-10.

10. A method according to claim 1, wherein the protractor group is selected from the group consisting of: dendrimer, polyalkylene oxide (PAO), polyalkylene glycol (PAG), polyethylene glycol (PEG), polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, dextran, carboxymethyl-dextran; serum protein binding-ligands, compounds which bind to albumin; a structure which inhibits the glycans from binding to receptors; a small organic molecule containing moieties that under physiological conditions alters charge properties; a low molecular organic charged radical of C1 to C25, which may optionally contain one or more carboxylic acids, amines sulfonic, phosphonic acids, or combination thereof; a low molecular neutral hydrophilic molecule; polyethyleneglycol with a avarage molecular weight of 2-40 KDa; and a substantially non-imunogenic polypeptide.

11. A method according to claim 1, wherein the glycoprotein is selected from the group consisting of: FVII, FVIII, FIX, FX, FII, FV, protein C, protein S, tPA, PAI-1, tissue factor, FXI, FXII, FXIII, sequence variants of any of the foregoing; immunoglobulins, cytokines such as interleukins, alpha-, beta-, and gamma-interferons, colony stimulating factors, platelet derived growth factors, phospholipase-activating protein (PUP), insulin, lectins, ricins, tumor necrosis factors, soluble forms of tumor necrosis factor receptors, interleukin receptors and soluble forms of interleukin receptors, tissue growth factors epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, and tissue plasminogen activator.

12. A glycoprotein conjugate having increased in vivo plasma half-life compared to the non-conjugated glycoprotein, said conjugate comprising a glycoprotein having at least one terminal galactose or derivative thereof, and a protractor group covalently bonded thereto, optionally through a linking moiety.

13. The glycoprotein conjugate according to claim 12, wherein the protractor group is selected from the group consisting of: dendrimer, polyalkylene oxide (PAO), polyalkylene glycol (PAG), polyethylene glycol (PEG), polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, dextran, carboxymethyl-dextran; serum protein binding-ligands, compounds which bind to albumin; a structure which inhibits the glycans from binding to receptors; a small organic molecule containing moieties that under physiological conditions alters charge properties; a low molecular organic charged radical of C1 to C25, which may optionally contain one or more carboxylic acids, amines sulfonic, phosphonic acids, or combination thereof; a low molecular neutral hydrophilic molecule; polyethyleneglycol with a avarage molecular weight of 2-40 KDa; and a substantially non-imunogenic polypeptide.

14. The glycoprotein conjugate according to any of claims 12 or 13, wherein the glycoprotein is selected from the group of: FVII, FVIII, FIX, FX, FII, FV, protein C, protein S, tPA, PAI-1, tissue factor, FXI, FXII, FXIII, sequence variants of any of the foregoing; immunoglobulins, cytokines such as interleukins, alpha-, beta-, and gamma-interferons, colony stimulating factors, platelet derived growth factors, phospholipase-activating protein (PUP), insulin, lectins, ricins, tumor necrosis factors, soluble forms of tumor necrosis factor receptors, interleukin receptors and soluble forms of interleukin receptors, tissue growth factors epidermal growth factors, hormones, somatomedins, erythropoietin, pigmentary hormones, hypothalamic releasing factors, antidiuretic hormones, prolactin, chorionic gonadotropin, follicle-stimulating hormone, thyroid-stimulating hormone, and tissue plasminogen activator.

15. A glycoprotein conjugate obtainable by a method according to claim 1.

16. A pharmaceutical composition comprising a glycoprotein conjugate according to claim 12.


SEQUENCE LISTING (1 sequence)

SEQ ID NO: 1 - protein, 406 aa, Human Coagulation Factor VII
  MISC_FEATURE (6)..(7): Xaa=Gla
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP